SEQUENCE LISTING (75 sequences)

SEQ ID NO: 1 - protein, 6 aa, Artificial sequence
  Non-natural sequence
GSGGGA

SEQ ID NO: 2 - protein, 12 aa, Artificial sequence
  Non-natural sequence
GSGGGAGSGGGA

SEQ ID NO: 3 - protein, 18 aa, Artificial sequence
  Non-natural sequence
GSGGGAGSGGGAGSGGGA

SEQ ID NO: 4 - protein, 24 aa, Artificial sequence
  Non-natural sequence
GSGGGAGSGGGAGSGGGAGSGGGA

SEQ ID NO: 5 - protein, 10 aa, Artificial sequence
  Non-natural sequence
GENLYFQSGG

SEQ ID NO: 6 - protein, 12 aa, Artificial sequence
  Non-natural sequence
KPEPKPAPAPKP

SEQ ID NO: 7 - protein, 12 aa, Artificial sequence
  Non-natural sequence
AEAAAKEAAAKA

SEQ ID NO: 8 - protein, 8 aa, Artificial sequence
  Non-natural sequence
SACYCELS

SEQ ID NO: 9 - protein, 10 aa, Artificial sequence
  Non-natural sequence
SGGGSSGGGS

SEQ ID NO: 10 - protein, 10 aa, Artificial sequence
  Non-natural sequence
GGGGSGGGGS

SEQ ID NO: 11 - protein, 10 aa, Artificial sequence
  Non-natural sequence
SSGGGSSGGG

SEQ ID NO: 12 - protein, 10 aa, Artificial sequence
  Non-natural sequence
GGSGGGGSGG

SEQ ID NO: 13 - protein, 10 aa, Artificial sequence
  Non-natural sequence
GSGSGSGSGS

SEQ ID NO: 14 - DNA, 20 bp, Artificial sequence
  Non-natural sequence
ggggtcaacgttgagggggg

SEQ ID NO: 15 - DNA, 20 bp, Artificial sequence
  Non-natural sequence
gggggacgatcgtcgggggg

SEQ ID NO: 16 - DNA, 21 bp, Artificial sequence
  Non-natural sequence
ggggacgacgtcgtggggggg

SEQ ID NO: 17 - DNA, 20 bp, Artificial sequence
  Non-natural sequence
tccatgacgttcctgatgct

SEQ ID NO: 18 - DNA, 20 bp, Artificial sequence
  Non-natural sequence
tccatgacgttcctgacgtt

SEQ ID NO: 19 - DNA, 24 bp, Artificial sequence
  Non-natural sequence
tcgtcgttttgtcgttttgtcgtt

SEQ ID NO: 20 - DNA, 22 bp, Artificial sequence
  Non-natural sequence
tcgtcgttgtcgttttgtcgtt

SEQ ID NO: 21 - DNA, 23 bp, Artificial sequence
  Non-natural sequence
tcgacgttcgtcgttcgtcgttc

SEQ ID NO: 22 - DNA, 26 bp, Artificial sequence
  Non-natural sequence
tcgcgacgttcgcccgacgttcggta

SEQ ID NO: 23 - DNA, 22 bp, Artificial sequence
  Non-natural sequence
tcgtcgttttcggcgcgcgccg

SEQ ID NO: 24 - DNA, 25 bp, Artificial sequence
  Non-natural sequence
tcgtcgtcgttcgaacgacgttgat

SEQ ID NO: 25 - DNA, 29 bp, Artificial sequence
  Non-natural sequence
tcgcgaacgttcgccgcgttcgaacgcgg

SEQ ID NO: 26 - DNA, 75 bp, Artificial sequence
  Non-natural sequence
agatctaacgacaaaacgacaaaacgacaaggcgccagatctggcgtttcgttttgtcgttttgtcgttagatct

SEQ ID NO: 27 - protein, 20 aa, Artificial sequence
  Non-natural sequence
GGTGCATCGATGCAGGGGGG

SEQ ID NO: 28 - protein, 83 aa, Artificial sequence
  Non-natural sequence
GGTGCATCGATGCAGGGGGGTATATATATATTGAGGACAGGTTAAGCTCCCCCCAGCTTAACCTGTCCTTCAATATATATATA

SEQ ID NO: 29 - DNA, 4479 bp, Artificial sequence
  Non-natural sequence
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctttttccctcgccaaaaattatggggacatcatgaagccccttgagcatctgacttctggctaataaaggaaatttatttcattgcaatagtgtgttggaattttttgtgtctctcactcggaaggacatatgggagggcaaatcatttaaaacatcagaatcagtatttggtttagagtttggcaacatatgccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 30 - DNA, 10 bp, Artificial sequence
  Non-natural sequence
cgccgccacc

SEQ ID NO: 31 - DNA, 36 bp, Artificial sequence
  Non-natural sequence
gagctcaaaaccccacttggtgacacaactcacaca

SEQ ID NO: 32 - DNA, 45 bp, Artificial sequence
  Non-natural sequence
gagcccaaatcttgtgacacacctcccccgtgcccaaggtgccca

SEQ ID NO: 33 - DNA, 30 bp, Artificial sequence
  Non-natural sequence
ggcggtggaagcagcggaggtggaagtgga

SEQ ID NO: 34 - DNA, 321 bp, Artificial sequence
  Non-natural sequence
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 35 - DNA, 15 bp, Artificial sequence
  Non-natural sequence
ggcctcggtggcctg

SEQ ID NO: 36 - DNA, 276 bp, Artificial sequence
  Non-natural sequence
atgaaggtctccaccactgcccttgctgttcttctctgtaccatgacactctgcaaccaagtcttctcagcgccatatggagctgacaccccgactgcctgctgcttctcctacagccggaagattccacgccaattcatcgttgactattttgaaaccagcagcctttgctcccagccaggtgtcattttcctgactaagagaaaccggcagatctgcgctgactccaaagagacctgggtccaagaatacatcactgacctggaactgaatgcc

SEQ ID NO: 37 - DNA, 270 bp, Artificial sequence
  Non-natural sequence
atgaagctctgcgtgtctgccctctctctcctcttgctcgtggctgccttctgtgctccagggttctcagcaccaatgggctctgaccctcccacttcctgctgtttctcttacacctcccggcagcttcacagaagctttgtgatggattactatgagaccagcagtctttgctccaagccagctgtggtattcctgaccaaaagaggcagacagatctgtgctaaccccagtgagccctgggtcactgagtacatgagtgacttggag

SEQ ID NO: 38 - DNA, 273 bp, Artificial sequence
  Non-natural sequence
atgaagatctctgcagctgccctcaccatcatcctcactgcagccgccctctgcacccccgcacctgcctcaccatatggctcggacaccactccctgctgctttgcctacctctccctcgcgctgcctcgtgcccacgtcaaggagtatttctacaccagcagcaagtgctccaatcttgcagtcgtgtttgtcactcgaaggaaccgccaagtgtgtgccaacccagagaagaagtgggttcaagaatacatcaactatttggagatgagc

SEQ ID NO: 39 - DNA, 324 bp, Artificial sequence
  Non-natural sequence
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtct

SEQ ID NO: 40 - DNA, 342 bp, Artificial sequence
  Non-natural sequence
atgagacttctcctcctgactttcctgggagtctgctgcctcaccccatgggttgtggaaggtgtggggactgaagtcctagaagagagtagctgtgtgaacttacaaacccagcggctgccagttcaaaaaatcaagacctatatcatctgggagggggccatgagagctgtaatttttgtcaccaaacgaggactaaaaatttgtgctgatccagaagccaaatgggtgaaagcagcgatcaagactgtggatggcagggccagtaccagaaagaacatggctgaaactgttcccacaggagcccagaggtccaccagcacagcgataaccctgactggg

SEQ ID NO: 41 - DNA, 291 bp, Artificial sequence
  Non-natural sequence
atggcctgcggtggcaagcgtctgctcttccttgctttggcatgggtactgctggctcacctctgcagccaggcagaagcagcaagcaactacgactgttgcctctcgtacatacagacgcctcttccttccagagctattgtgggtttcacaagacagatggccgatgaagcttgtgacattaatgctatcatctttcacacgaagaaaagaaaatctgtgtgcgctgatccaaagcagaactgggtgaaaagggctgtgaacctcctcagcctaagagtcaagaagatg

SEQ ID NO: 42 - DNA, 399 bp, Artificial sequence
  Non-natural sequence
atggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagagga

SEQ ID NO: 43 - DNA, 423 bp, Artificial sequence
  Non-natural sequence
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaa

SEQ ID NO: 44 - DNA, 55 bp, Artificial sequence
  Non-natural sequence
atgaaatgggtgacctttctgctgctgctgtttgtgagcggcagcgcgtttagcg

SEQ ID NO: 45 - DNA, 785 bp, Artificial sequence
  Non-natural sequence
cggcccagccggccatggcggactacaaacaggctgttgtgactcaggaatcagcactcaccacatcacctggtgaaacagtcacactcacttgtcgctcaagtactggggctgttacaattagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatctatttctgtgctctatggtacaacaaccagtttattttcggcagtggaaccaaggtcactgtcctaggtggtggtggtggttctggtggtggtggatccggcggcggcggctctggcggcggcggctctgaggtccagctgcaacagtctggacctgtgctggtgaagcctggggcttcagtgaagatgtcctgtaaggcttctggaaacacattcactgactcctttatgcactggatgaaacagagccatggaaagagtcttgagtggattggaattattaatccttataacggcggtactagctacaaccagaaattcaagggcaaggccacattgactgttgacaagtcctccagcacagcctacatggagctcaacagcctgacatctgaggactctgcagtctattactgtgcaagaaacggggtgcggtactactttgactactggggccaaggcaccactctcacagtctcctcggcctcgggggcc

SEQ ID NO: 46 - DNA, 831 bp, Artificial sequence
  Non-natural sequence
gcggcccagccggccatggcggactacaaacaggctgttgtgactcaggaatcagcactcaccacatcacctggtgaaacagtcacactcacttgtcgctcaagtaaaagcagccagagcgtgctgtatgatgaaaacaaaaaaaactatctggccaactgggtccaagaaaaaccagatcatttattcactggtctaataggttgggcgagcaccggcgaaagcaaccgagctccaggtgttcctgccagattctcaggctccctgattggagacaaggctgccctcaccatcacaggggcacagactgaggatgaggcaatctatttctgttattatgattttccgccgttcggcagtggaaccaaggtcactgtcctaggtggtggtggtggttctggtggtggtggatccggcggcggcggctctggcggcggcggctctgaggtccagctgcaacagtctggacctgtgctggtgaagcctggggcttcagtgaagatgtcctgtaaggcttctaacgcggcgatttatatgcactggatgaaacagagccatggaaagagtcttgagtggattggaattcgcattcgcacccgcccgagcaaatatgcgaccgattatgcggatagcgtgcgcggcagctacaaccagaaattcaagggcaaggccacattgactgttgacaagtcctccagcacagcctacatggagctcaacagcctgacatctgaggactctgcagtctattactgtcgcgcgaccgaagatgtgccgttttattactggggccaaggcaccactctcacagtctcctcggcctcgggggcc

SEQ ID NO: 47 - protein, 36 aa, Artificial sequence
  Non-natural sequence
TGGCCCAGGTTCCACAGCAGCGTGTTCCACACCCAC

SEQ ID NO: 48 - DNA, 489 bp, Artificial sequence
  Non-natural sequence
atgacacctgactgttacttcagccacagtcccatctcctccaacttcaaagtgaagtttagagagttgactgaccacctgcttaaagattacccagtcactgtggccgtcaatcttcaggacgagaagcactgcaaggccttgtggagcctcttcctagcccagcgctggatagagcaactgaagactgtggcagggtctaagatgcaaacgcttctggaggacgtcaacaccgagatacattttgtcacctcatgtaccttccagcccctaccagaatgtctgcgattcgtccagaccaacatctcccacctcctgaaggacacctgcacacagctgcttgctctgaagccctgtatcgggaaggcctgccagaatttctctcggtgcctggaggtgcagtgccagccggactcctccaccctgctgcccccaaggagtcccatagccctagaagccacggagctcccagagcctcggcccaggcag

SEQ ID NO: 49 - DNA, 105 bp, Artificial sequence
  Non-natural sequence
ggagaactggaggaattacttaaacatctcaaggagttgctcaaaggccctaggaagggagaactggaggaactcctcaaacatctcaaggagttactaaaggga

SEQ ID NO: 50 - DNA, 30 bp, Artificial sequence
  Non-natural sequence
ggcagcggcagcggcagcggcagcggcagc

SEQ ID NO: 51 - DNA, 333 bp, Homo sapiens
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 52 - DNA, 180 bp, Artificial sequence
  Non-natural sequence
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 53 - DNA, 117 bp, Artificial sequence
  Non-natural sequence
aagcctctggacggagagtatttcactctccagatccggggccccgaaaggttcgaaatgttccgggagcttaacgaggccttggagctgaaagacgcacaggccggaaaggaaccg

SEQ ID NO: 54 - protein, 27 aa, Artificial sequence
  Non-natural sequence
QIETQQRKFKASRASILSEMKMLKEKR

SEQ ID NO: 55 - protein, 27 aa, Artificial sequence
  Non-natural sequence
VILPQAPSGPSYATYLQPAQAQMLTPP

SEQ ID NO: 56 - protein, 27 aa, Artificial sequence
  Non-natural sequence
DTLSAMSNPRAMQVLLQIQQGLQTLAT

SEQ ID NO: 57 - protein, 27 aa, Artificial sequence
  Non-natural sequence
DGQLELLAQGALDNALSSMGALHALPR

SEQ ID NO: 58 - protein, 27 aa, Artificial sequence
  Non-natural sequence
RLHVVKLLASALSTNAAALTQELLVLD

SEQ ID NO: 59 - protein, 12 aa, Homo sapiens
ELKTPLGDTTHT

SEQ ID NO: 60 - protein, 15 aa, Homo sapiens
EPKSCDTPPPCPRCP

SEQ ID NO: 61 - protein, 27 aa, Artificial sequence
  Non-natural sequence
QIETQQRKFKASRASILSEMKMLKEKR

SEQ ID NO: 62 - protein, 27 aa, Artificial sequence
  Non-natural sequence
VILPQAPSGPSYATYLQPAQAQMLTPP

SEQ ID NO: 63 - protein, 27 aa, Artificial sequence
  Non-natural sequence
DTLSAMSNPRAMQVLLQIQQGLQTLAT

SEQ ID NO: 64 - protein, 27 aa, Artificial sequence
  Non-natural sequence
DGQLELLAQGALDNALSSMGALHALPR

SEQ ID NO: 65 - protein, 27 aa, Artificial sequence
  Non-natural sequence
RLHVVKLLASALSTNAAALTQELLVLD

SEQ ID NO: 66 - protein, 5 aa, Artificial sequence
  Non-natural sequence
GGGSS

SEQ ID NO: 67 - protein, 27 aa, Artificial sequence
  Non-natural sequence
GEVPPQKLQALQRALQSEFCNAVREVY

SEQ ID NO: 68 - protein, 21 aa, Artificial sequence
  Non-natural sequence
KKFMERDPDELRFNTIALSAA

SEQ ID NO: 69 - protein, 27 aa, Artificial sequence
  Non-natural sequence
VTGTHKMSLGFTKARLLRLRNPWGRVE

SEQ ID NO: 70 - protein, 27 aa, Artificial sequence
  Non-natural sequence
LWTFSIYLESVAIMPQLFMVSKTGEAE

SEQ ID NO: 71 - protein, 27 aa, Artificial sequence
  Non-natural sequence
LHSGQNHLKEMAISVLEARACAAAGQS

SEQ ID NO: 72 - protein, 27 aa, Artificial sequence
  Non-natural sequence
GSLFGSSRVQYVVNPAVKIVFLNIDPS

SEQ ID NO: 73 - protein, 27 aa, Artificial sequence
  Non-natural sequence
PPPGLAAYTAKMATANGSKKAERQKFS

SEQ ID NO: 74 - protein, 27 aa, Artificial sequence
  Non-natural sequence
VCNVKLLHRVLVADVNALQGMAAIGQR

SEQ ID NO: 75 - DNA, 4099 bp, Artificial sequence
  Recombinant vector backbone
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctaacgacaaaacgacaaaacgacaaggcgccagatctggcgtttcgttttgtcgttttgtcgttagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

(19) United States
(12) Patent Application Publication
(10) Pub. No.: US 2023/0147574 A1
(43) Pub. Date: May 11, 2023

(54) Neoepitope immunotherapy with APC targeting unit

(71) Applicant: Evaxion Biotech A/S, Hørsholm (DK)

(72) Inventors: Birgitte Rønø, Hørsholm (DK); Marina Barrio Calvo, Hørsholm (DK); Anders Bundgård Sørensen, Hørsholm (DK); Christian Thygesen, Hørsholm (DK); Jens Kringelum, Hørsholm (DK); Stine Friis, Hørsholm (DK)

(21) Appl. No.: 17/995,601

(22) PCT Filed: Apr. 7, 2021
(86) PCT No.: PCT/EP2021/059117

§ 371 Date: Oct. 6, 2022

(30) Foreign Application Priority Data
Apr. 7, 2020 (EP) 20168405.7
Nov. 13, 2020 (EP) 20207528.9

(51) Int. Cl. C07K 14/74; A61K 39/00; C12N 15/62; A61K 39/39; A61P 35/00; C07K 14/52; C07K 16/28; C07K 14/78; C07K 14/47

(52) U.S. Cl. CPC C07K 14/70539; A61K 39/0011; C12N 15/62; A61K 39/39; A61P 35/00; C07K 14/521; C07K 16/2851; C07K 14/78; A61K 39/001151; C07K 14/4746; A61K 39/001196; C07K 2319/33; A61K 2039/505; A61K 38/00

(57) ABSTRACT

The present invention relates to cancer therapy, in particular cancer immunotherapy. In particular, the present invention relates to methods and products for treating cancer by administration of specific fusion polypeptides or nucleic acids encoding such fusion polypeptides.

### FIELD OF THE INVENTION

The present invention relates to cancer therapy, in particular cancer immunotherapy. In particular, the present invention relates to methods and products for treating cancer by administration of specific fusion polypeptides or nucleic acids encoding such fusion polypeptides.

### BACKGROUND OF THE INVENTION

Treatment of malignant neoplasms in patients has traditionally focussed on eradication/removal of the malignant tissue via surgery, radiotherapy, and/or chemotherapy using cytotoxic drugs in dosage regimens that aim at preferential killing of malignant cells compared to killing of non-malignant cells.

In addition to the use of cytotoxic drugs, more recent approaches have focussed on targeting of specific biologic markers in the cancer cells in order to reduce systemic adverse effects exerted by classical chemotherapy. Monoclonal antibody therapy targeting cancer associated antigens has proven quite effective in prolonging life expectance in a number of malignancies. While being successful drugs, monoclonal antibodies that target cancer associated antigens or antigen can by their nature only be developed to target expression products that are known and appear in a plurality of patients, meaning that the vast majority of cancer specific antigens cannot be addressed by this type of therapy, because a large number of cancer specific antigens only appear in tumours from one single patient, cf. below.

As early as in the late 1950′ies the theory of immunosurveillance proposed by Burnet and Thomas suggested that lymphocytes recognize and eliminate autologous cells—including cancer cells—that exhibit altered antigenic determinants, and it is today generally accepted that the immune system inhibits carcinogenesis to a high degree. Nevertheless, immunosurveillance is not 100% effective and it is a continuing task to device cancer therapies where the immune system's ability to eradicate cancer cells is sought improved/stimulated.

One approach has been to induce immunity against cancer-associated antigens, but even though this approach has the potential of being promising, it suffers the same drawback as antibody therapy that only a limited number of antigens can be addressed.

Many if not all tumours express mutations. These mutations potentially create new targetable antigens (neo-antigens), which are potentially useful in specific T cell immunotherapy if it is possible to identify the neo-antigens and their antigenic determinants within a clinically relevant time frame. Since it with current technology is possible to fully sequence the genome of cells and to analyse for existence of altered or new expression products, it is possible to design personalized vaccines based on neo-antigens. However, attempts at providing satisfactory clinical results have as of today largely failed.

One mode of vaccination that has been investigated in detail since the early 1990′ies is nucleic acid vaccination (also termed DNA vaccination), where DNA is administered in a non-viral plasmid form to somatic cells of a mammal leading to expression of the genes comprised in the plasmid; in DNA vaccination the encoded material is immunogenic polypeptide(s), which upon production by the somatic cells will be able to induce an immune response. This approach is appealing as it avoids the need of producing the protein immunogen in clinical grade purity using expensive recombinant expression systems. However, it has proven difficult to obtain expression levels from the DNA administered which are high enough to effect satisfactory immune responses in humans.

Antigen-presenting cells (APC) are vital for effective adaptive immune response and are cells that display antigen complexed with major histocompatibility complexes (MHCs) on their surfaces. The cells include macrophages, B cells and dendritic cells, and present foreign antigens to helper T cells. Also, virus-infected cells or cancer cells can present antigens originating inside the cell to cytotoxic T cells. Consequently, targeting antigen-presenting cell offers opportunities to induce superior immune responses.

There is an existing need for provision of anti-cancer vaccines, in particular nucleic acid vaccines that can effectively target neo-antigens and induce clinically significant immune responses in vaccinated human beings.

### OBJECT OF THE INVENTION

It is an object of embodiments of the invention to provide polypeptides constructs and nucleic acid molecules encoding such polypeptide constructs with superior anti-tumor effect and which constructs are able to elicit even higher T cell responses than known vaccines.

It is a further object of embodiments of the invention to provide polypeptides constructs and nucleic acid molecules encoding such polypeptide constructs designed to direct and assist in APC uptake of cancer neo-epitopes, optionally activation of the APC followed by cytokine cascade enabling the provision of a very effective protective immunity against the cancer.

It is a further object of embodiments of the invention to provide methods for the use of such polypeptides constructs and nucleic acid molecules encoding such polypeptide constructs.

### SUMMARY OF THE INVENTION

The present inventors have designed novel and improved polypeptides constructs and nucleic acid molecules encoding such polypeptide constructs generating a next generation DNA neoepitope immunotherapy with an antigen presenting cell (APC) -targeting unit.

It has been found by the present inventor(s) that targeting of APC may be used to enhance the immunotherapeutic effects while maintaining antitumor activity of a neo-epitope vaccine, which enable a superior anti-tumor effect eliciting even higher T cell responses than known vaccines.

So, in a first aspect the present invention relates to fusion polypeptides comprising

i) at least one antigenic unit, which comprises a sequence of amino acids of at least one neo-epitope of the patient's neoplastic cells;

ii) at least one antigen presenting cell (APC) targeting unit;

iii) optionally a multimerization, such as a dimerization unit, which unit provides for the multimerization of said fusion polypeptide to comprise two or more antigenic units and two or more antigen presenting cell (APC) targeting units.

In a second aspect the present invention relates to an expression vector, which comprises a sequence of nucleotides encoding a fusion polypeptide according to the present invention.

In a further aspect the present invention relates to a system of at least two expression constructs comprising i) a first expression construct comprising a sequence of nucleotides encoding at least one antigenic unit, which antigenic unit comprises a sequence of amino acids of at least one neo-epitope of the patient's neoplastic cells, and ii) a second expression construct comprising a sequence of nucleotides encoding at least one antigen presenting cell (APC) targeting unit.

In a further aspect the present invention relates to a method for the treatment of a neoplasm, such as a malignant neoplasm or for inducing a therapeutic or ameliorating immune response against such neoplasm, in a mammalian patient, wherein the neoplasm exhibits T-cell epitopes (neo-epitopes) that are not exhibited by non-neoplastic cells in the patient, the method comprising administering an immunogenically effective amount of a composition comprising a fusion polypeptide according to the present invention, or which composition is comprising at least one expression vector which comprises a sequence of nucleotides encoding a fusion polypeptide according to the present invention, whereby somatic cells in the patient are brought to express the sequence of nucleotides contained within the expression vector; the method optionally further comprising administering a pharmaceutically acceptable carrier, diluent, or excipient. In some embodiments the patient is a human being. In some embodiments the immunogenically effective amount of a composition is administered parenterally, such as via the intramuscular route, the intradermal route, transdermal route, the subcutaneous route, the intravenous route, the intra-arterial route, the intratechalintrathecal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

In some embodiments the pharmaceutically acceptable carrier, diluent, or excipient is an aqueous buffered solution. In some embodiments the aqueous buffered solution is Tyrode's buffer. In some embodiments the Tyrode's buffer has the composition 140 mM NaCl, 6 mM KCl, 3 mM CaCl₂), 2 mM MgCl2, 10 mM 4-(2-hydroxyethyl)-1-piperazineethanesulfonic acid (Hepes) pH 7.4, and 10 mM glucose. In some embodiments the concentration of Tyrode's buffer is about 35% v/v. In some embodiments the aqueous buffer is phosphate-buffered saline (PBS) buffer.

In some embodiments the method comprises administering an immunogenically effective amount of a composition comprising at least one expression vector as defined in any one of claims 10-15 with an effective dosage between 0.1 μg and 25 mg of the expression vector, such as between 0.5 μg and 20 mg, between 5 μg and 15 mg, between 50 μg and 10 mg, and between 500 μg and 8 mg, in particular about 0.0001, about 0.0005, about 0.001, about 0.005, about 0.01, about 0.05, about 0.1, about 0.5, about 1, about 2, about 3, about 4, about 5, about 6, about 7 and about 8 mg.

In some embodiments the method comprises administering an immunogenically effective amount of a composition which composition further comprises an effective amount of an amphiphilic block co-polymers comprising blocks of poly(ethylene oxide) and polypropylene oxide), such as Kolliphor® P188.

### LEGENDS TO THE FIGURE

FIG. 1: Illustration of one suggested design of a suitable fusion polypeptide and DNA encoding it. In this example the construct contains an APC targeting unit being CCL3 or other cytokine/chemokine; a dimerization unit containing Hinge (h1 and h4) and CH3 from IgG3; and antigenic units being neo-epitopes.

FIG. 2: Illustration of mechanism of action for the DNA vaccine as illustrated in FIG. 1.

FIG. 3: Illustration of different APC targeting units for fusion polypeptide designs according to the invention. A) illustrates the constructs of example 1, whereas B) illustrates the constructs of example 2.

FIG. 4: Plasmid map of pUMVC4 from Aldevron.

Details are provided in the examples and SEQ ID NO:29.

FIG. 5: Plasmid map of pUMVC4 mCCL19 S16A as example of an APC targeting design. The pUMVC4 vector is containing an insert encoding a kozak sequence, murine CCL19 as APC targeting unit, hinge 1, hinge 4 and Ch3 from human IgG followed by the 5 neoepitopes C22, C23, C38, C25, C30.

FIG. 6: Tumour volume reduction in mice vaccinated with Kolliphor and vaccine plasmids. APC targeting vaccines as compared to control groups (untreated mice or mice treated with empty mock plasmid). *: p<0.05 (Kruskal-Wallis test). See example 1 for details.

FIG. 7: Diagram shows detection of C22 MHC I multimers.

The graph shows the frequency of murine CD8+T cells reactive with the C22 peptide upon vaccination of mice with experimental DNA vaccines. See example 1 for details.

FIG. 8: IFN-γ production in T cells from mice vaccinated with Kolliphor and vaccine plasmids. See example 1 for details.

FIG. 8A: Intracellular cytokine staining (ICS on stimulated splenocytes). See example 1 for details.

FIG. 9: Tumour volume reduction in mice vaccinated with Kolliphor and vaccine plasmids. See example 2 for details.

FIG. 10: Diagram shows detection of C22 MHC I multimers.

The graph shows the frequency of murine CD8+T cells reactive with the C22 peptide upon vaccination of mice with experimental DNA vaccines. See example 2 for details.

FIG. 11: IFN-γ production in T cells from mice vaccinated with Kolliphor® and vaccine plasmids. See example 2 for details.

FIG. 12: Tumour volume reduction in mice vaccinated with Kolliphor® and vaccine plasmids. See example 3 for details

FIG. 13: Diagram shows detection of C22 MHC I multimers.

The graph shows the frequency of murine CD8+T cells reactive with the C22 peptide-loaded MHCI tetramer upon vaccination of mice with experimental DNA vaccines. See example 3 for details.

FIG. 14: IFN-γ production in T cells from mice vaccinated with Kolliphor® and vaccine plasmids. See example 3 for details.

FIG. 15: Illustration of DNA designs encoding the separate units of the fusion protein according to the invention. Illustrates the constructs of example 4.

FIG. 16: Tumour volume reduction in mice vaccinated with Kolliphor® and vaccine plasmids. See example 4 for details

FIG. 17: Diagram shows detection of C22 MHC I multimers. The graph shows the frequency of murine CD8+T cells reactive with the C22 peptide-loaded MHCI tetramer upon vaccination of mice with experimental DNA vaccines. See example 4 for details.

FIG. 18: DNA designs according to the invention.

FIG. 19: Results from restimulation of splenic cells with 27 mer neopeptides.

A: Schematic depiction of constructs used for immunization.
B: Graph shown percentage of INFy and TNFα producing CD8+ cells upon restimulation of splenocytes.
C: Graph shown percentage of INFy and TNFα producing CD4+ cells upon restimulation of splenocytes.

FIG. 20: Schematic representation of designs for selected APC targeting candidates.

FIG. 21: Plasmid maps of pTVG4 based constructs

A: Empty backbone vector.
B: Empty backbone vector with inserted NotI and BamHi restriction sites and murine CCL19 encoding sequence
C: Vector as in B, but with coding region for neoepitopes inserted between NotI and BamHI sites—this construct is also termed mEVX-03.
D: Vector as in C, but with human CCL19 encoding sequence instead of murine CCL19 encoding sequence.

FIG. 22: Schematic overview of DNA cassettes tested in Example 5.

FIG. 23: Graphs showing tumour growth in mice immunized with the constructs tested in Example 5.

A: Graph showing area under curve (AUC) for tumour volume in treated groups.
B: Graph showing change in tumour volume in treated groups relative to control). Asterisks indicate a p value of <0.05 in a Kruskal-Wallis analysis.

FIG. 24: Graphs showing percentage of C22 specific CD8+ T cells in whole blood at days 2 (A) and day 6 (B) in treated groups from Example 5.

FIG. 25: Plasmid map of mEVX-02 used for comparison in Example 6.

FIG. 26: Graphs comparing tumour volumes in mice immunized with different doses of plasmid.

A: Immunization with mEVX-02.
B: Immunization with mEVX-03.
C: Area-under-curve (AUC) comparisons with empty plasmid immunization.

FIG. 27: Graphs showing dose titration of EVX-02 and -03.

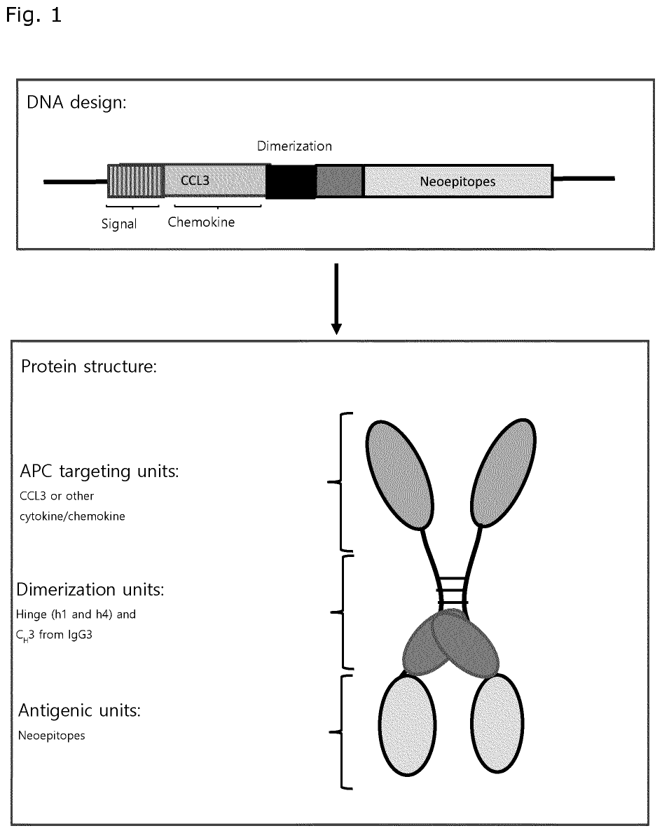

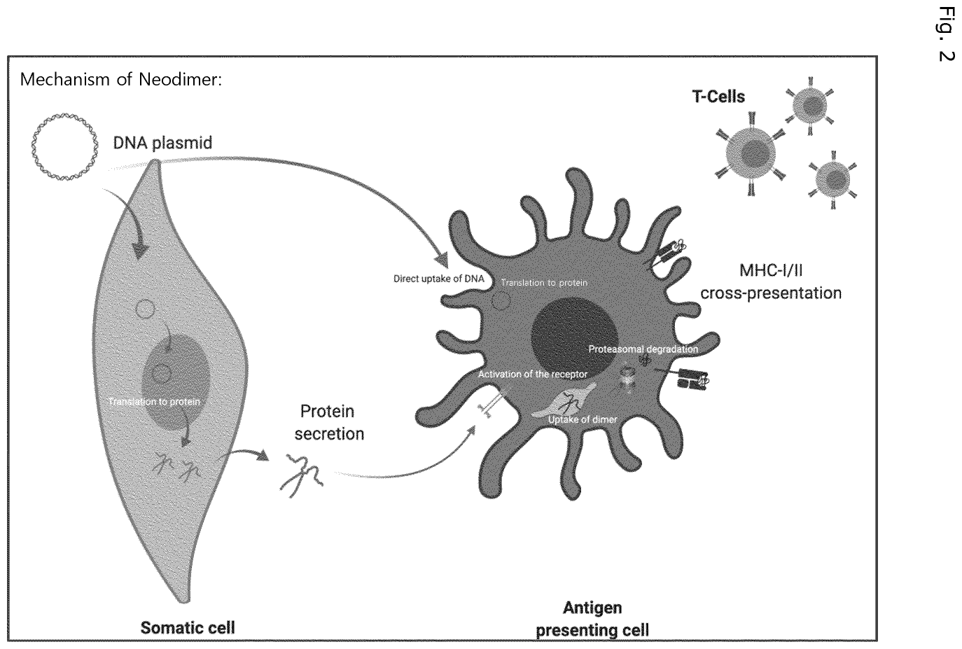

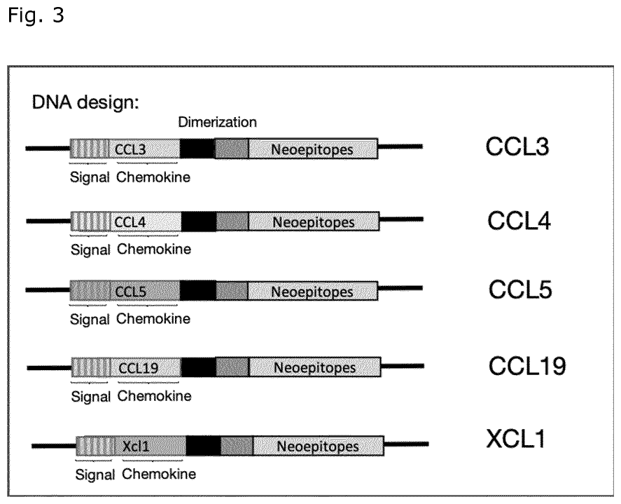

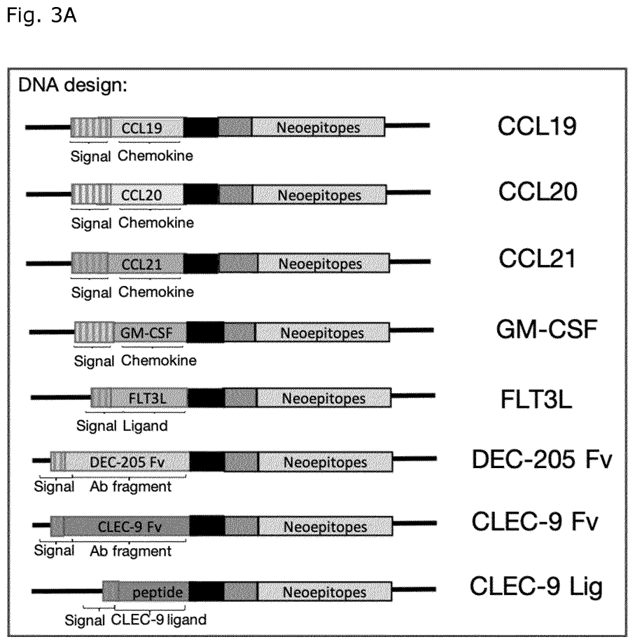

A: Relative to tumour reduction.

### B: Relative to AUC.

FIG. 28: Graphs showing induction of CD8+ T cells in whole blood by EVX-02 and -03.

A: On day 6 post challenge.
B: On day 16 post challenge.

FIG. 29: Graphs showing dose titration of EVX-02 and -03 relative to CD8 cell induction.

A: On day 6 post challenge.
B: On day 16 post challenge.

### DETAILED DISCLOSURE OF THE INVENTION

A DNA neo-epitope immunotherapy containing an APC targeting element is expected to have superior anti-tumor effect and may elicit higher T cell responses, than a DNA technology without, due to either i) directing and assisting in APC uptake of the neo-epitopes, and/or ii) activation of the APC followed by cytokine cascade.

The fusion polypeptide constructs according to the present invention comprise epitopes (neo-epitopes), such as T cell epitopes that are not exhibited by non-neoplastic cells in the patient.

A “neo-epitope” is an antigenic determinant (typically an MHC Class I or II restricted epitope), which does not exist as an expression product from normal somatic cells in an individual due to the lack of a gene encoding the neo-epitope, but which exists as an expression product in mutated cells (such as cancer cells) in the same individual. As a consequence, a neo-epitope is from an immunological viewpoint truly non-self in spite of its autologous origin and it can therefore be characterized as a tumour specific antigen in the individual, where it constitutes an expression product. Being non-self, a neo-epitope has the potential of being able to elicit a specific adaptive immune response in the individual, where the elicited immune response is specific for antigens and cells that harbour the neo-epitope. Neo-epitopes are on the other hand specific for an individual as the chances that the same neo-epitope will be an expression product in other individuals is minimal. Several features thus contrast a neo-epitope from e.g. epitopes of tumour specific antigens: the latter will typically be found in a plurality of cancers of the same type (as they can be expression products from activated oncogenes) and/or they will be present—albeit in minor amounts—in non-malignant cells because of over-expression of the relevant gene(s) in cancer cells.

A “neo-peptide” is a peptide (i.e. a polyamino acid of up to about 50 amino acid residues), which includes within its sequence a neo-epitope as defined herein. A neo-peptide is typically “native”, i.e. the entire amino acid sequence of the neo-peptide constitutes a fragment of an expression product that can be isolated from the individual, but a neo-peptide can also be “artificial”, meaning that it is constituted by the sequence of a neo-epitope and 1 or 2 appended amino acid sequences of which at least one is not naturally associated with the neo-epitope. In the latter case the appended amino acid sequences may simply act as carriers of the neo-epitope, or may even improve the immunogenicity of the neo-epitope (e.g. by facilitating processing of the neo-peptide by antigen-presenting cells, improving biologic half-life of the neo-peptide, or modifying solubility).

A “neo-antigen” is any antigen, which comprises a neo-epitope. Typically, a neo-antigen will be constituted by a protein, but a neo-antigen can, depending on its length, also be identical to a neo-epitope or a neo-peptide.

The term “amino acid sequence” is the order in which amino acid residues, connected by peptide bonds, lie in the chain in peptides and proteins. Sequences are conventionally listed in the N to C terminal direction.

The fusion polypeptide constructs according to the present invention further comprise at least one antigen presenting cell (APC) targeting unit.

Antigen-presenting cells (APC) are cells that displays antigen complexed with major histocompatibility complexes (MHCs) on their cell surfaces, a process known as antigen presentation. Specialized antigen-presenting cells include macrophages, B cells and dendritic cells, which present foreign antigens to helper T cells, while virus-infected cells (or cancer cells) can present antigens originating inside the cell to cytotoxic T cells. An antigen presenting cell (APC) targeting unit is any molecule or ligand that is suitable for the specific targeting to these APC, such as by specifically targeting different surface molecules on APCs.

Suitable targeting units to be used according to the invention includes the following as well as the corresponding human sequence:

| Molecule | Function | SEQ ID |
|---|---|---|
| mCCL3 | APC targeting | SEQ ID NO: 36 |
| mCCL4 | APC targeting | SEQ ID NO: 37 |
| mCCL5 | APC targeting | SEQ ID NO: 38 |
| mCCL19 | APC targeting | SEQ ID NO: 39 |
| mXcl1 | APC targeting | SEQ ID NO: 40 |
| mCCL20 | APC targeting | SEQ ID NO: 41 |
| mCCL21 | APC targeting | SEQ ID NO: 42 |
| GM-CSF | APC targeting | SEQ ID NO: 43 |
| Secretion signal, mouse | secretion signal | SEQ ID NO: 44 |
| serum albumin | | |
| anti-murine DEC-205 Fv | APC targeting | SEQ ID NO: 45 |
| anti-murine CLEC9 Fv | APC targeting | SEQ ID NO: 46 |
| CLEC9 ligand | APC targeting | SEQ ID NO: 47 |
| mFLT3L | APC targeting | SEQ ID NO: 48 |

Suitable targeting units to be used according to the invention is disclosed in any one of Takashi Sato et al. Blood. 2011 Mar 24; 117(12): 3286-3293; Cagan Gurer et al. Blood. 2008 Aug 15; 112(4): 1231-1239; Wan-Lun Yan et al. Immunotherapy (2017) 9(4), 347-360; Gerty Schreibelt et al. BLOOD, 8 Mar. 2012, VOLUME 119, NUMBER 10; and Zhongyi Yan et al. Oncotarget, Vol. 7, No. 26, May 2016, p. 40437.

### Definitions

As used herein a “linker” refers to any compound suitable for assembly of the two or more different or identical linear peptide sequences or subunits into a multimeric polypeptide. The term includes any linker found useful in peptide chemistry. Since the multimeric polypeptide or fusion polypeptide may be assembled or connected by standard peptide bonds in a linear way, the term linker also includes a “peptide spacer”, also referred to as a “spacer”. It is to be understood that linkers may be used both to separate encoded neo-epitopes in the fusion polypeptides of the invention, or linkers may be used to separate the neo-epitope units of the fusion polypeptide from the antigen presenting cell (APC) targeting unit of the fusion polypeptide.

A linker may be “rigid”, meaning that it does substantially not allow the two amino acid sequences that it connects to move freely relative to each other. Likewise, a “flexible” linker allows the two sequences connected via the linker to move substantially freely relative to each other. In encoded expression products that contain more than one neo-epitope, both types of linkers are useful.

Linkers of interest, which can be encoded by an expression vector used in the invention, are listed in the following table:

| Type | Name | Sequence |
|---|---|---|
| Flexible | FS | GSGGGA |
| | | (SEQ ID NO: 1) |
| Flexible | FL | GSGGGAGSGGGA |
| | | (SEQ ID NO: 2) |
| Flexible | FV1 | GSGGGAGSGGGAGSGGGA |
| | | (SEQ ID NO: 3) |
| Flexible | FV2 | GSGGGAGSGGGAGSGGGAGSGGGA |
| | | (SEQ ID NO: 4) |
| Flexible | FM | GENLYFQSGG |
| | | (SEQ ID NO: 5) |
| Rigid | RL1 | KPEPKPAPAPKP |
| | | (SEQ ID NO: 6) |
| Rigid | RL2 | AEAAAKEAAAKA |
| | | (SEQ ID NO: 7) |
| Rigid | RM | SACYCELS |
| | | (SEQ ID NO: 8) |
| Flexible | | SGGGSSGGGS |
| | | (SEQ ID NO: 9) |
| Flexible | | GGGGSGGGGS |
| | | (SEQ ID NO: 10) |
| Flexible | | SSGGGSSGGG |
| | | (SEQ ID NO: 11) |
| Flexible | | GGSGGGGSGG |
| | | (SEQ ID NO: 12) |
| Flexible | | GSGSGSGSGS |
| | | (SEQ ID NO: 13) |
| Hinge | H1 | ELKTPLGDTTHT |
| | | (SEQ ID NO: 59) |
| Hinge | H4 | EPKSCDTPPPCPRCP |
| | | (SEQ ID NO: 60) |

In some embodiments, the linker is a peptide sequence. In some embodiments, the linker is not a peptide sequence. In some embodiments, the linker is not a branched peptide sequence.

In some embodiments, the linker does not itself contain a peptide sequence derived from or identical to the neo-epitope sequence and/or the antigen presenting cell (APC) targeting unit.

In some embodiments, the linker is derived from an immunoglobulin molecule (Ig), such as from IgG.

In some embodiments, the linker is or comprises a hinge region, such as a flexible hinge region, such as a hinge region derived from an immunoglobulin molecule (Ig), such as from IgG.

Alternative suitable linkers to be used according to the invention:

| Molecule | Function | SEQ ID NO: |
|---|---|---|
| dHLX protein | dimerization | SEQ ID NO: 49 |
| hMHD2 (human IgM) | dimerization | SEQ ID NO: 51 |
| Collagen trimerisation domain | Trimerization | SEQ ID NO: 52 |
| p53 synthetic protein | Tetramerization | SEQ ID NO: 53 |

Accordingly, in some embodiments the linker comprises or consists of a hinge region derived from IgM, and/or comprises or consists of a dimerization motif derived from a sequence encoded by SEQ ID NO:51.

In some embodiments the linker comprises or consists of a trimerisation domain, such as a Collagen trimerisation domain, such as a trimerisation domain derived from a sequence encoded by SEQ ID NO:52.

In some embodiments the linker comprises or consists of a dimerization motif derived from hMHD2 or dHXL, optionally further comprising a hinge region such as H1 described herein.

In some embodiments the linker comprises or consists of a tetramerization domain, such as a domain derived from p53, such as a tetraimerization domain derived from a sequence encoded by SEQ ID NO:53, optionally further comprising a hinge region such as H1 described herein.

Suitable linkers to be used according to the invention is also described in any one of: Ana Alvarez-Cienfuegos et al, Scientific Reports 2016, 6:28643 I DOI: 10.1038/srep28643; Victor J. Sanchez-Arevalo Lobo et al. Int. J. Cancer: 119, 455-462 (2006); Oliver Seifert et al. Protein Engineering, Design & Selection vol. 25 no. 10 pp. 603-612, 2012; and Jorg Willuda et al. THE JOURNAL OF BIOLOGICAL CHEMISTRY Vol. 276, No. 17, Issue of April 27, pp. 14385-14392, 2001.

“An immunogenic carrier” or “pharmaceutically acceptable carrier” as used herein is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the antigens bound to MHC molecules are being recognized by T-cell receptors) but which are not necessarily capable of inducing immunity -an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “polypeptide” is in the present context intended to mean both short peptides of from 2 to 50 amino acid residues, oligopeptides of from 50 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “fusion polypeptide” is in the present context intended to mean a polypeptide containing polypeptide elements or amino acid sequences having intended different functions.

The different polypeptide elements or amino acid sequences are connected through a linker, which may be just another amino acid sequence, or the different polypeptide elements or amino acid sequences of the fusion polypeptide may just be connected by standard peptide bonds in a linear way.

The expression vector used according to the present invention is typically and preferably comprised in or constituted by a plasmid, but other expression vectors can be employed. The composition of the present invention aims at ensuring delivery of “naked” DNA to cells, i.e. a DNA expression vector, which is not part of a bacterium or virus that would be able to effect introduction of the expression vector into the target cells. A vector useful in the present compositions and methods can thus be circular or linear, single-stranded or double stranded and can in addition to a plasmid also be e.g. a cosmid, mini-chromosome or episome.

Each coding (and expressible) region can be present on the same or on separate vectors; however, it is to be understood that one or more coding regions can be present on a single vector, and these coding regions can be under the control of a single or multiple promoters.

This means that the expression vector can encode a separate peptide expression product for each encoded fusion polypeptide or that the expression vector can encode a plurality of peptide expression products, where at least some exhibit(s) several encoded fusion polypeptide, of which at least some optionally are separated by peptide linkers.

In other words, in some cases only one single expression vector is administered and expressed, and this expression vector may encode a plurality of separate proteinaceous expression products or as few as 2 or even one single expression product—in this context it is only relevant whether the encoded fusion polypeptide are satisfactorily presented to the immune system and the choice of their presence in separate on combined expression products is therefore of minor relevance. In preferred embodiments, the expression vector expresses at least or about 5, such as at least or about 10, at least or about 15, at least or about 20, at least or about 25, at least or about 30 proteinaceous expression products. Higher numbers are contemplated and the limit is primarily set by the number of neo-epitopes of the fusion polypeptides it is possible to identify from a particular neoplasm. It goes without saying that the number of encoded neo-epitopes of the fusion polypeptide in the expression vector(s) cannot exceed the number of neo-epitopes found in the relevant malignant tissue.

The use of peptide linkers to separate encoded neo-epitope expression products provides spatial separation between epitopes in the expression product of the fusion polypeptide. This can entail several advantages: linkers can ensure that each neo-epitope is presented in an optimized configuration to the immune system, but use of appropriate linkers can also minimize the problem that irrelevant immune responses are directed against “junctional epitopes” which emerge in the regions constituted by the C-terminal end of one neo-epitope and the adjacent N-terminal end of the next neo-epitope in a multi-epitope containing expression product.

Encoded peptide linkers can be either “flexible” or “rigid”, cf. the definition above, where preferred encoded linkers are set forth. Also, it is envisaged that the linker(s) used in the invention in some embodiments can be cleavable, that is, include (a) recognition site(s) for endopeptidase(s), e.g. endopeptidases such as furin, caspases, cathepsins etc.

The neo-epitopes encoded by the expression vector can be identified in a manner known per se: “deep sequencing” of the genome of the malignant cells and of the genome of healthy cells in the same individual or a standard healthy genome can identify expressed DNA sections that provide for potentially immunogenic expression products unique to the malignant cells. The identified DNA sequences can thereafter be codon-optimized (typically for expression by human cells) and included in the expression vector—either as separate expression regions of as part of larger chimeric constructs.

In order to optimize the identification and selection of the neo-epitopes that are to be expressed by the vector, any of the prediction methods available for this purpose are in practice useful. One example of a state of the art prediction algorithm is NetMHCpan-4.0 (www.cbs.dtu.dk/services/NetMHCpan-4.0/; Jurtz V et al., J Immunol (2017), ji1700893; DOI: 10.4049/jimmuno1.1700893). This method is trained on a combination of classical MS derived ligands and pMHC affinity data. Another example is NetMHCstabpan-1.0 (www.cbs.dtu.dk/services/NetMHCstabpan-1.0/; Rasmussen M et al., Accepted for J of Immunol, June 2016). This method is trained on a dataset of in vitro pMHC stability measurement using an assay where each peptide is synthesized and complexed to the MHC molecule in vitro. No cell processing is involved in this assay and the environment where the pMHC stability is measured is somewhat artificial. The method is in general less accurate than NetMHCpan-4.0. U.S. Pat. No. 10,055,540 describes a method for identification of neo-epitopes using classical MS detected ligands. Other patent application publications using similar technology are WO 2019/104203, WO 2019/075112, WO 2018/195357 (MHC Class II specific), and WO 2017 106638. Finally, MHCflurry: (DOI: doi.org/10.1016/j.cels.2018.05.014; https://github.com/openvax/mhcflurry) is like NetMHCpan trained on MS detected ligand data and pMHC affinities. A peptide-MHC Class II interaction prediction method is also disclosed in a recent publication Garde C et al., Immunogenetics, DOI: doi.org/10.1007/s00251-019-01122-z. In this publication, naturally processed peptides eluted from MHC Class II are used as part of the training set and assigned the binding target value of 1 if verified as ligands and 0 if negative.

Generally, these prediction systems employ artificial neural networks (ANNs): ANNs can identify non-linear correlations: Quantification of non-linear correlations is not an easy task, since it is difficult to calculate by simple calculation. This is primarily due to non-linear correlations described with more parameters than linear correlations and probably first appear when all features are considered collectively. Hence it is needed to take all features into account in order to catch the dependency across features.

In order to further improve the likelihood that the selected encoded neo-epitopes provide for an effective immune response, use can preferably be made of the technology disclosed in European patent application nos: 19197295.9 and 19197306.4, both filed on 13 Sep. 2019. These applications disclose technology, which enables that stability of binding between peptides and MHC molecules can be determined and which enables that stability of MHC binding of neo-epitopes is determined as part of the neo-epitope detection and selection. In brief, the data obtained from stability determinations are e.g. used as part of the training set for an ANN, and the ANN can subsequently rank identified peptides according to their predicted binding stabilities towards relevant MHC molecules.

When a nucleic acid vaccine is administered to a patient, the corresponding gene product (such as a desired antigen) is produced in the patient's body. In some embodiments, nucleic acid vaccine vectors that include optimized recombinant polynucleotides can be delivered to a human to induce a therapeutic or prophylactic immune response.

Plasmid and other DNA vectors are typically more efficient for gene transfer to muscle tissue. The potential to deliver DNA vectors to mucosal surfaces by oral administration has also been reported and DNA plasmids have been utilized for direct introduction of genes into other tissues than muscle. DNA vaccines have been introduced into animals primarily by intramuscular injection, by gene gun delivery, by jet injection (using a device such as a Stratis® device from PharmaJet), or by electroporation; each of these modes of administration apply to the presently disclosed method. After being introduced, the plasmids are generally maintained episomally without replication. Expression of the encoded proteins has been shown to persist for extended time periods, providing stimulation of both B and T cells.

In determining the effective amount of the vector to be administered in the treatment method disclosed herein, the physician evaluates vector toxicities, progression of the cancer to be treated, and the production of anti-vector antibodies, if any. Administration can be accomplished via single or divided doses and typically as a series of time separated administrations. In the methods disclosed herein, the effective human dose per immunization in a time-separated series is between 0.1 μg and 500 mg, with dosages between 0.1 μg and 25 mg of the expression vector being preferred. That is, in the practice of the method disclosed herein dosages of between 0.5 μg and 20 mg in humans are typically used, and dosages are normally between 5 μg and 15 mg, between 50 μg and 10 mg, and between 500 μg and 8 mg, and particular interesting dosages are of about 0.0001, about 0.0005, about 0.001, about 0.005, about 0.01, about 0.05, about 0.1, about 0.5, about 1, about 2, about 3, about 4, about 5, about 6, about 7 and about 8 mg.

A series of immunizations with effective dosages will typically constitute a series of 2, 3, 4, 5, 6, or more dosages. Multiple (e.g. >6) dosages may for instance be relevant in order to keep a malignant neoplasm in check for a prolonged period and in such a situation the exact choice of encoded neo-epitopes in the vaccine vector can be changed over time in response to changes in the genome and proteome of the malignant cells. When and if new neo-epitopes are produced by the malignant cells these can conveniently be included as targets for the vaccine.

The vaccine used in the method disclosed herein comprises one or more expression vectors; for instance, the vaccine may comprise a plurality of expression vectors each capable of autonomous expression of a nucleotide coding region in a mammalian cell to produce at least one immunogenic polypeptide. An expression vector often includes a eukaryotic promoter sequence, such as the nucleotide sequence of a strong eukaryotic promoter, operably linked to one or more coding regions. The compositions and methods herein may involve the use of any particular eukaryotic promoter, and a wide variety are known; such as a CMV or RSV promoter. The promoter can be heterologous with respect to the host cell. The promoter used may be a constitutive promoter. The promoter used may include an enhancer region and an intron region to improve expression levels, such as is the case when using a CMV promoter.

Numerous plasmids known in the art may be used for the production of nucleic acid vaccines. Suitable embodiments of the nucleic acid vaccine employ constructs using the plasmids VR1012 (Vical Inc., San Diego Calif.), pCMVI.UBF3/2 (S. Johnston, University of Texas), pTVG4 (Johnson et al., 2006, Vaccine 24(3); 293-303), pVAX1 (Thermo Fisher Scientific, see above and the Examples below), or pcDNA3.1 (InVitrogen Corporation, Carlsbad, Calif.) as the vector.

In addition, the vector construct can according to the present invention advantageously contain immunostimulatory sequences (ISS). The aim of using such sequences in the vaccine vector is to enhance T-cell response towards encoded neo-epitopes, in particular Th1 cell responses, which are elicited by adjuvants that incorporate agonists of the toll-like receptors TLR3, TLR7-TLR8, and TLR9. and/or cytosolic RNA receptors such as, but not limited to, RIG-1, MDAS and LGP2 (Desmet et al. 2012. Nat. Rev. Imm. 12(7), 479-491)

One possibility of employing ISS is to mimic a bacterial infection activating TLR9 by stimulating with unmethylated CG-rich motifs (so-called CpG motifs) of six bases with the general sequence NNCGNN (which have a 20-fold higher frequency in bacterial DNA than in mammalian DNA) either as directly administered small synthetic DNA oligos (ODNs), which contain partially or completely phosphorothioated backbones, or by incorporating the CpG motifs in the DNA vector backbone. Immunostimulatory CpGs can be part of the DNA backbone or be concentrated in an ISS where the CpG sequence(s) typically will be positioned between the stop codon in the neo-epitope coding sequence and the poly-A tail encoding sequence (i.e. the ISS is located between the stop codon and the polyadenylation signal). However, since CpG sequences exert an effect irrespectively of their position in a longer DNA molecule, their position could in principle be anywhere in the vaccine vector as long as the presence of the CpG motif does not interfere with the vector's ability to express the coding regions of the vaccine antigen.

If present in the vaccine as separate ODNs, where the ODNs function as immunological adjuvants, CpG motif containing oligonucleotides are typically to be co-administered/formulated together with the DNA vaccine by the selected delivery technology and will typically constitute hexamers or longer multimers of DNA comprising the sequence NNCGNN or the reverse complementary sequence. Useful ODNs for this purpose are e.g.

commercially available from InivoGen, 5 Rue Jean Rodier, F-31400, Toulouse, France, which markets a range of Class A, B, and C ODNs. Examples are:

ODN1585
SEQ ID NO: 14
(5′-ggGGTCAACGTTGAgggggg-3′),
ODN2216
SEQ ID NO: 15
(5′-ggGGGACGATCGTCgggggg-3′),
ODN2336
SEQ ID NO: 16
(5′-gggGACGACGTCGTGgggggg-3′),
ODN1668
SEQ ID NO: 17
(5′-tccatgacgttcctgatgct-3′),
ODN1826
SEQ ID NO: 18
(5′-tccatgacgttcctgacgtt-3′),
ODN2006
SEQ ID NO: 19
(5′-tcgtcgttttgtcgttttgtcgtt-3′),
ODN2007
SEQ ID NO: 20
(5′-tcgtcgttgtcgttttgtcgtt-3′),
ODNBW006
SEQ ID NO: 21
(5′-tcgacgttcgtcgttcgtcgttc-3′),
ODN D-SL01
SEQ ID NO: 22
(5′-tcgcgacgttcgcccgacgttcggta-3′),
ODN2395
SEQ ID NO: 23
(5′-tcgtcgttttcggcgcgcgccg-3′),
ODN M362
SEQ ID NO: 24
(5′-tcgtcgtcgttcgaacgacgttgat-3′),
ODN D-SL03
SEQ ID NO: 25
(5′-tcgcgaacgttcgccgcgttcgaacgcgg-3′),

In these 12 ODNs, upper case nucleotides are phosphodiesters, lower case nucleotides are phosphorothioates, and underlining denotes palindromic sequences.

When CpG sequences are present in the plasmid backbone (which thereby become “self-adjuvating”), any number of possible NNGCNN or NNCGNN sequences can according to the invention be present, either as identical sequences or in the form of non-identical sequences of the CpG motif, or in the form of palindromic sequences that can form stem-loop structures. For instance, the following CpG motifs are of interest: AACGAC and GTCGTT, but also CTCGTT, and GCTGTT. An example of the use of such CpG encoding sequences is the following sequence excerpt from the commercially available pTVG4 vaccine vector backbone

(SEQ ID NO: 26)
. . . agatctaacgacaaaacgacaaaacgacaaggcgccagatct
ggcgtttcgttttgtcgttttgtcgttagatct . . .,

where the underlined nucleotides constitute the CpGs that are present in the pTVG4 plasmid vector sequence.

Another possibility is to mimic an RNA viral infection to activate TLR3 by adding a double stranded(ds) RNA either as synthetic RNA oligos such as Poly I:C (polyinosinic-polycytidylic acid), poly IC:U12 (uridine substituted poly I:C), or in the form of synthetic RNA oligonucleotides (ORNs); addition of these RNA molecules to the vaccine is, as for the ODN approach, a way of obtaining an adjuvant effect. Alternatively, the dsRNA can be encoded in the DNA vector backbone, which will be transcribed into RNA after vaccination—in this case the DNA vaccine hence encodes the immunological adjuvant. This approach can include DNA sequences that encode hairpin RNA with lengths of up to 100 base pairs, where the sequence is unspecific. Also the DNA can simultaneously include ODNs and encode ORNs of known sequences; the DNA can thus both be transcribed into a double stranded RNA capable of activating TLR3 and/or cytosolic RNA receptors such as RIG-1, MDAS, and LGP2 while comprising an ODN to activate TLR9. Examples of specific DNA sequences that include/encode immune stimulating CpG and dsRNA are for instance 5′-GGTGCATCGA TGCAGGGGGG-3′ (SEQ ID NO:27) and 5′-GGTGCATCGA TGCAGGGGGG TATATATATA TTGAGGACAG GTTAAGCTCC CCCCAGCTTA ACCTGTCCTT CAATATATA TATA-3′ (SEQ ID NO:28) (ref: Wu et al.2011, Vaccine 29(44): 7624-30).

When ISS are present in the DNA vaccine vector, it is possible—and advantageous - to combine the approach of using CpG motifs to activate TLR9 with the presence of coding sequences for immune stimulating RNA to activate TLR3 and/or cytosolic RNA receptors such as RIG-1, MDAS, and LGP2; cf. Grossmann C et al. 2009, BMC. Immunology 10:43 and Desmet et al. 2012. Nat. Rev. Imm. 12(7), 479-491. Likewise, incorporation of ORNs and ODNs in the vaccine as separate adjuvants (alone or in combination) may be combined with the incorporation of ISS of both types in the DNA vaccine vector.

As is the case for the CpG motifs, the DNA encoding the immune stimulatory RNA ISS will preferably be present between the stop codon and the polyadenylation signal but can be present in any part of the vector as long as this does not impair the production of the intended polypeptide expression product.

In some specific embodiments, ISS is/are comprised in the vaccine compositions, and in particular embodiments this is achieved by incorporating an immunologically active and pharmaceutically acceptable amount of poly I:C and/or poly IC:U12. Poly I:C is constituted by a mismatched double-stranded RNA (dsRNA) with one strand being a polymer of inosinic acid and the other strand a polymer of cytidylic acid. Poly IC:U12 is a variant of poly I:C where uridine is introduced into the Poly I:C strand. These two substances will in that context function as immunological adjuvants, i.e. substances that themselves do not elicit a specific adaptive immune response, but which enhances the specific adaptive immune response against the vaccine antigen (or in the present case, the encoded antigen).

Poly I:C or poly IC:U12 (such as Ampligen®) will preferably be present in the composition so as to arrive at an administered dosage of between 0.1 and 20 mg per administration of the effective dosage of the expression vector; that is, the amount present in the composition is adjusted so as to arrive at such dosages per administration. Preferably the administered dosage of poly I:C or poly IC:U12 is between 0.2 and 15 mg per administration of the effective dosage of the expression vector, such as between 0.3 and 12, 0.4 and 10 and 0.5 and 8 mg, preferably about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9 and 4.0 mg. Particularly preferred are in the range between 0.5 and 2.0 mg per administration.

In some specific embodiments the vaccine composition may comprise a amphiphilic block co-polymers comprising blocks of poly(ethylene oxide) and polypropylene oxide), such as poloxamers, i.e. nonionic triblock copolymers composed of a central hydrophobic chain of polyoxypropylene (poly(propylene oxide)) flanked by two hydrophilic chains of polyoxyethylene (poly(ethylene oxide)). One particular preferred amphiphilic block co-polymers is poloxamer 188 (Kolliphor® P188 from BASF). The administered dosage of amphiphilic block co-polymers may be between 0.2% w/v and 20% w/v per administration of the effective dosage of the expression vector, such as between 0.2% w/v and 18% w/v, such as between 0.2% w/v and 16% w/v, such as between 0.2% w/v and 14% w/v, such as between 0.2% w/v and 12% w/v, such as between 0.2% w/v and 10% w/v, such as between 0.2% w/v and 8% w/v, such as between 0.2% w/v and 6% w/v, such as between 0.2% w/v and 4% w/v, such as between 0.4% w/v and 18% w/v, such as between 0.6% w/v and 18% w/v, such as between 0.8% w/v and 18% w/v, such as between 1% w/v and 18% w/v, such as between 2% w/v and 18% w/v, such as between 1% w/v and 5% w/v, such as between 2% w/v and 4% w/v. Particularly preferred are in the range between 0.5% w/v and 5% w/v per administration.

Accordingly, the vaccine composition according to the present invention, such as a DNA vaccine composition may comprises a pharmacologically acceptable amphiphilic block co-polymer comprising blocks of poly(ethylene oxide) and polypropylene oxide, which is described in detail in the following:

The amphiphilic block co-polymer is described more generally under the definition heading, but the preferred the amphiphilic block co-polymer is a poloxamer or a poloxamine. Poloxamers only vary slightly with respect to their properties, but preferred are poloxamer 407 and 188, in particular poloxamer 188.

When the amphiphilic block co-polymer is poloxamine, the preferred type is a sequential poloxamine of formula (PEO-PPO)4-ED, where PEO is poly(ethylene oxide), PPO is poly(propylene oxide) and ED is an ethylenediaminyl group. These molecules attain an X-like shape where the PEO-PPO groups protrude from the central ethylenediaminyl group. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

When used in the method disclosed herein, the concentration of the amphiphilic block co-polymer in the vaccine composition may preferably between 2 and 5% w/v, such as about 3% w/v.

A “PEO-PPO” or amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO—PPO-PEO (“poloxamers”), PPO PEO PPO, (PEO PPO-)4ED (a “poloxamine”), and (PPO PEO-)4ED (a “reverse poloxamine”), where “ED” is a ethylenediaminyl group.

A “poloxamer” is a linear amphiphilic block copolymer constituted by one block of poly(ethylene oxide) (“PEO”) coupled to one block of poly(propylene oxide) (“PPO”) coupled to one block of PEO, i.e. a structure of the formula EOa-POb-EOa, where EO is ethylene oxide, PO is propylene oxide, a is an integer ranging between 2 and 130, and b is an integer ranging between 15 and 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of Mwc≈J1800 (corresponding to b≈31 PPO) and approximately 80% (w/w) of PEO (corresponding to a≈82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) are X-shaped block copolymers that bear four PEO-PPO arms connected to a central ethylenediamine via bonds between the free OH groups in the PEO-PPO-groups and the primary amine groups in ethylenediamine, and “reverse poloxamine are likewise X-shaped block copolymers that bear four PPO-PEO arms connected to a central ethylenediamine via bonds between the free OH groups in the PPO-PEO-groups and the primary amine groups in ethylenediamine.

The nucleic acid vaccine can also encode a fusion product containing one or more immunogenic polypeptides containing neo-epitopes. Plasmid DNA can also be delivered using attenuated bacteria as delivery system, a method that is suitable for DNA vaccines that are administered orally. Bacteria are transformed with an independently replicating plasmid, which becomes released into the host cell cytoplasm following the death of the attenuated bacterium in the host cell.

DNA vaccines, including the DNA encoding the desired antigen, can be introduced into a host cell in any suitable form including, the fragment alone, a linearized plasmid, a circular plasmid, a plasmid capable of replication, an episome, RNA, etc. Preferably, the gene is contained in a plasmid. In certain embodiments, the plasmid is an expression vector. Individual expression vectors capable of expressing the genetic material can be produced using standard recombinant techniques.

Routes of administration include, but are not limited to, intramuscular, intranasal, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. In other words, the route of administration can be selected from any one of parenteral routes, such as via the intramuscular route, the intradermal route, transdermal route, the subcutaneous route, the intravenous route, the intra-arterial route, the intrathecal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

Typical routes of administration include intramuscular, intraperitoneal, intradermal and subcutaneous injection. Genetic constructs may be administered by means including, but not limited to, traditional syringes, needleless injection devices, “microprojectile bombardment gene guns”, or other physical methods such as electroporation (“EP”), “hydrodynamic method”, or ultrasound. DNA vaccines can be delivered by any method that can be used to deliver DNA as long as the DNA is expressed and the desired antigen is made in the cell.

In some embodiments, a DNA vaccine composition disclosed herein is delivered via or in combination with known transfection reagents such as cationic liposomes, fluorocarbon emulsion, cochleate, tubules, gold particles, biodegradable microspheres, or cationic polymers. Cochleate delivery vehicles are stable phospholipid calcium precipitants consisting of phosphatidyl serine, cholesterol, and calcium; this nontoxic and noninflammatory transfection reagent can be present in a digestive system. Biodegradable microspheres comprise polymers such as poly(lactide-co-glycolide), a polyester that can be used in producing microcapsules of DNA for transfection. Lipid-based microtubes often consist of a lipid of spirally wound two layers packed with their edges joined to each other. When a tubule is used, the nucleic acid can be arranged in the central hollow part thereof for delivery and controlled release into the body of an animal.

A DNA vaccine can also be delivered to mucosal surfaces via microspheres. Bioadhesive microspheres can be prepared using different techniques and can be tailored to adhere to any mucosal tissue including those found in eye, nasal cavity, urinary tract, colon and gastrointestinal tract, offering the possibilities of localized as well as systemic controlled release of vaccines. Application of bioadhesive microspheres to specific mucosal tissues can also be used for localized vaccine action. In some embodiments, an alternative approach for mucosal vaccine delivery is the direct administration to mucosal surfaces of a plasmid DNA expression vector which encodes the gene for a specific protein antigen.

The DNA plasmid vaccines disclosed are formulated according to the mode of administration to be used. Typically, the DNA plasmid vaccines are injectable compositions, they are sterile, and/or pyrogen free and/or particulate free. In some embodiments, an isotonic formulation is preferably used. Generally, additives for isotonicity can include sodium chloride, dextrose, mannitol, sorbitol and lactose. In some embodiments, isotonic solutions such as phosphate buffered saline are preferred; one preferred solution is Tyrode's buffer. In some embodiments, stabilizers include gelatine and albumin. In some embodiments, a stabilizing agent that allows the formulation to be stable at room or ambient temperature for extended periods of time, such as LGS or other poly-cations or poly-anions is added to the formulation.

The second constituent in the composition disclosed herein is the pharmaceutically acceptable carrier, diluent, or excipient, which is preferably in the form of a buffered solution. Parenteral vehicles include sodium chloride solution, Ringer's dextrose and sodium chloride, lactated Ringer's or fixed oils. Intravenous vehicles include fluid and nutrient replenishers, electrolyte replenishers such as those based on Ringer's dextrose, and the like. Preservatives and antimicrobials include antioxidants, chelating agents, inert gases and the like. Preferred preservatives include formalin, thimerosal, neomycin, polymyxin B and amphotericin B.

In preferred embodiments, the buffered solution is the one known as “Tyrode's buffer”, and in preferred embodiments the Tyrode's buffer has the composition 140 mM NaCl, 6 mM KCl, 3 mM CaCl2), 2 mM MgCl2, 10 mM 4-(2-hydroxyethyl)-1-piperazineethanesulfonic acid (Hepes) pH 7.4, and 10 mM glucose. The concentration of the Tyrode's buffer (or alternatives) is typically about 35% v/v, but depending on the water content of suspended plasmids, the concentration may vary considerably—since the buffer is physiologically acceptable, it can constitute any percentage of the aqueous phase of the composition.

Furthermore, in preferred embodiments, the buffered solutions is the PBS, and in preferred embodiments the PBS has composition of composition 0.28 mg Potassium dihydrogen phosphate, 1.12 mg Disodium hydrogen phosphate dihydrate and 9.0 Sodium chloride per 1 ml solution.

Additional carrier substances may be included and can contain proteins, sugars, etc. Such carriers may be aqueous or non-aqueous solutions, suspensions, and emulsions. Examples of non-aqueous carriers are propylene glycol, polyethylene glycol, vegetable oils such as olive oil, and injectable organic esters such as ethyl oleate. Aqueous carriers include water, alcoholic/aqueous solutions, emulsions or suspensions, including saline.

Specific Embodiments of the Invention

As described above the present invention relates to fusion polypeptide comprising i) at least one antigenic unit, which comprises a sequence of amino acids of at least one neo-epitope of the patient's neoplastic cells; ii) at least one antigen presenting cell (APC) targeting unit; iii) optionally a multimerization, such as a dimerization unit, which unit provides for the multimerization of said fusion polypeptide to comprise two or more antigenic units and two or more antigen presenting cell (APC) targeting units.

In some embodiments the APC targeting unit consist of or comprises an antibody binding region with specificity for target surface molecules on antigen presenting cells, such as HLA, HLA-DP, CD14, CD40; or Toll-like receptor, such as Toll-like receptor 2; ligands, such as soluble CD40 ligand; CLEC9A Fv fragments, DEC205 Fv fragments, GM-CSF, natural ligands like chemokines, such as a chemokine of the CC chemokine family, such as any one selected from chemokine ligand 3, chemokine ligand 4, chemokine ligand 5, chemokine ligand 19, chemokine ligand 20, chemokine ligand 21, or similar; or a chemokine of the CXC chemokine family, such as any one selected from chemokine (C—X-C motif) ligand 1 (CXCL1), or similar, RANTES or bacterial antigens, such as flagellin or a part thereof.

In some embodiments the APC targeting unit consist of or comprises a ligand, such as soluble CD40 ligand; CLEC9A peptide ligand, DEC205 ligand, FLT3L, GM-CSF, natural ligands like chemokines, such as a chemokine of the CC chemokine family, such as any one selected from chemokine ligand 3, chemokine ligand 4, chemokine ligand 5, chemokine ligand 19, chemokine ligand 20, chemokine ligand 21, or similar; or a chemokine of the CXC chemokine family, such as any one selected from chemokine (C—X-C motif) ligand 1 (CXCL1), or similar, such as RANTES or Chemokine ligand 3 (CCL3/MIP-la) or CCL19; or bacterial antigens, such as flagellin or a part thereof.

In some embodiments the antigenic unit is connected to the targeting unit through a linker, such as GS linker, such as linker with the amino acid sequence GSGSGSGSGS (SEQ ID NO:13), or a linker derived from an immunoglobulin molecule (Ig), such as IgG, such as a linker which contributes to the multimerization through the formation of an interchain covalent bond. In some embodiments this linker is or comprises a hinge region, such as an Ig, such as an IgG-derived hinge region and contributes to the multimerization through the formation of an interchain covalent bond, such as a disulfide bridge. In some embodiments this linker comprises a carboxyterminal C domain (CH3 domain), such as the carboxyterminal C domain of Ig (Cy3 domain), or a sequence that is substantially homologous to said C domain, such as the CH3 domain of IgG3. In some embodiments this hinge and CH3 domain are connected by a sequence of amino acids GlyGlyGlySerSer (SEQ ID NO:66), such as in triplicate sequence of the amino acids GlyGlyGlySerSer. In some embodiments this linker comprises a dimerization motif or any other multimerization domain, which participate in the multimerization through hydrophobic interactions, such as through a CH3 domain. In some embodiments this linker comprises a hinge region comprising h1+h4 or h4 derived from IgG, such as an IgG2 or IgG3.

In some embodiments the at least one antigenic unit consist of or comprises at least or about 5, such as at least or about 10, at least or about 15, at least or about 20, at least or about 25, and at least or about 30 neo-epitopes.

In some embodiments the at least one neo-epitope includes a neo-epitope, which exhibits an MHC binding stability, which is above average, such as in the top quartile, among neo-epitopes identified in the neoplastic cells.

In some embodiments the multimerization, such as a dimerization unit, enables the formation of dimers, trimers, tetramers, pentamers, or multimers of higher order.

The present invention further relates to an expression vector, which comprises a sequence of nucleotides encoding a fusion polypeptide of the invention.

In some embodiments the sequence of nucleotides is a cDNA sequence.

In some embodiments the sequence of nucleotides is an RNA sequence encoding the fusion polypeptide of the invention.

In some embodiments the sequence further comprises or encodes at least one immune stimulating sequence (ISS). In some embodiments the ISS is an oligodeoxyribonucleotide (ODN) comprising at least one CpG motif, and wherein the ODN preferably includes phosphorothioate groups. In some embodiments the ISS is or comprises an oligoribonucleotide.

In some embodiments the sequence further comprises a secretion signal.

The present invention further relates to system of at least two expression constructs comprising i) a first expression construct comprising a sequence of nucleotides encoding at least one antigenic unit, which antigenic unit comprises a sequence of amino acids of at least one neo-epitope of the patient's neoplastic cells, and ii) a second expression construct comprising a sequence of nucleotides encoding at least one antigen presenting cell (APC) targeting unit.

In some embodiments the first expression construct comprising a sequence of nucleotides encoding at least one antigenic unit consist of or comprises at least or about 5, such as at least or about 10, at least or about 15, at least or about 20, at least or about 25, and at least or about 30 neo-epitopes.

In some embodiments the first expression construct comprising a sequence of nucleotides encoding least one neo-epitope, which includes a neo-epitope, which exhibits an MHC binding stability, which is above average, such as in the top quartile, among neo-epitopes identified in the neoplastic cells.

In some embodiments the second expression construct comprises a sequence of nucleotides encoding at least one antigen presenting cell (APC) targeting unit which consist of or comprises an antibody binding region with specificity for target surface molecules on antigen presenting cells, such as HLA, HLA-DP, CD14, CD40; or Toll-like receptor, such as Toll-like receptor 2; ligands, such as soluble CD40 ligand; CLEC9A Fv fragment, DEC205 Fv fragment, natural ligands like chemokines, such as a chemokine of the CC chemokine family, such as any one selected from chemokine ligand 3, chemokine ligand 4, chemokine ligand 5, chemokine ligand 19, chemokine ligand 20, chemokine ligand 21, or similar; or a chemokine of the CXC chemokine family, such as any one selected from chemokine (C—X-C motif) ligand 1 (CXCL1), or similar, RANTES or bacterial antigens, such as flagellin or a part thereof.

In some embodiments the second expression construct comprises a sequence of nucleotides encoding at least one antigen presenting cell (APC) targeting unit which consist of or comprises a ligand, such as soluble CD40 ligand; CLEC9A peptide ligand, DEC205, FLT3L, GM-CSF, natural ligands like chemokines, such as a chemokine of the CC chemokine family, such as any one selected from chemokine ligand 3, chemokine ligand 4, chemokine ligand 5, chemokine ligand 19, chemokine ligand 20, chemokine ligand 21, or similar; or a chemokine of the CXC chemokine family, such as any one selected from chemokine (C—X-C motif) ligand 1 (CXCL1), or similar, such as RANTES or Chemokine ligand 3 (CCL3/MIP-la) or CCL19; or bacterial antigens, such as flagellin or a part thereof.

In some embodiments the first expression construct comprising a sequence of nucleotides encoding at least one antigenic unit further comprises a sequence of nucleotides encoding a multimerization, such as a dimerization unit, which unit provides for the multimerization of said at least one antigenic unit.

In some embodiments the at least two expression constructs is expressed on the same expression vector, such as under the control of two different promotors.

In some embodiments the at least two expression constructs is expressed by at least two different vectors.

### EXAMPLE 1

Assessment of chemokines as APC targeting unit for delivery of neoepitopes

The objectives of the study were to test the ability of the invention, an APC targeting DNA vaccine, to induce neo-peptide specific T cells, reduce tumor growth and to monitor the impact of the vaccine on the wellbeing of vaccinated mice.

Plasmids for DNA vaccination were based on the commercially available pUMVC4™ vector available from Aldevron.

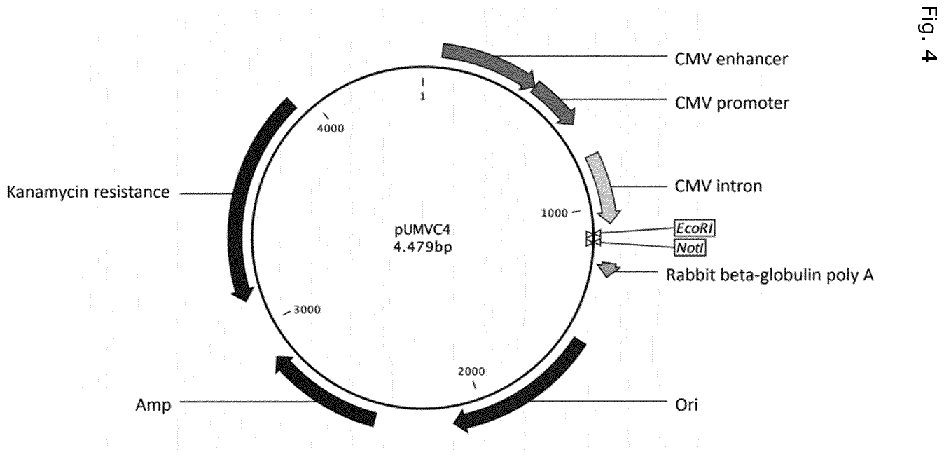

pUMVC4™ is according to the manufacturer's documentation a 4479 kb plasmid vector, which allows high-copy number replication in E. coli and high-level transient expression of encoded protein of interest in most mammalian cells. The vector (see FIG. 4) contains the following elements:

1) a human cytomegalovirus immediate-early (CMV) promoter for high-level expression in a wide range of mammalian cells,

2) a rabbit beta-globulin polyadenylation signal for efficient transcription termination and polyadenylation of mRNA,

3) a kanamycin resistance gene for selection in E. coli and

4) an immunostimulatory sequence (ISS) from ampicillin resistance gene, which makes it ideal for eliciting an immune response in vivo

The entire sequence of the pUMVC4™ plasmid is set forth in SEQ ID NO:29.

Five expression vectors generated with the pUMVC4 as backbone and an APC targeting unit guiding the neoepitopes in S16A were constructed. The various chemokines used as APC targeting units are listed in the following table:

| APC targeting unit | SEQUENCE ID NO |
|---|---|
| mCCL3 | SEQ ID NO: 36 |
| mCCL4 | SEQ ID NO: 37 |
| mCCL5 | SEQ ID NO: 38 |
| mCCL19 | SEQ ID NO: 39 |
| mXCL1 | SEQ ID NO: 40 |

The five S16A neoepitopes were first identified by whole exome sequencing of the mouse colon cancer cell line CT26 and normal tissue samples from BALB/c mice and by selecting peptides found only in the cancer cells. In the experiment, the ability of the mice to generate immune responses against the identified neoepitopes was evaluated.

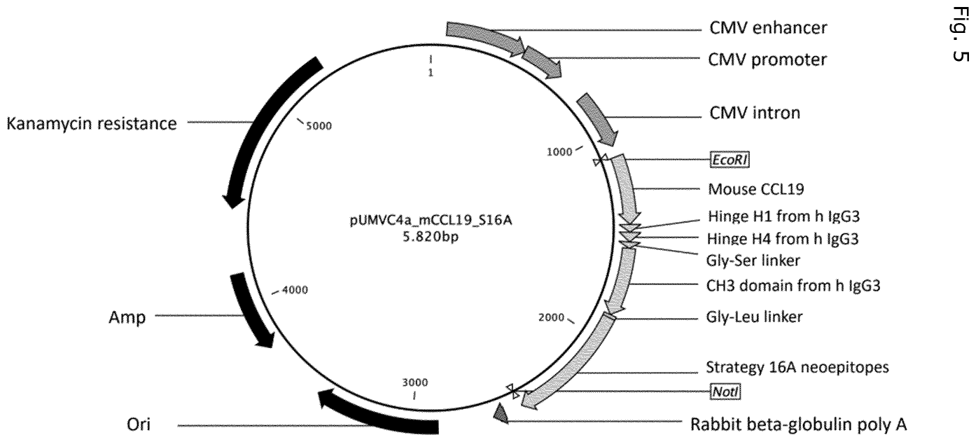

pUMVC4 APC-targeting S16A was constructed by ligating a DNA insert containing an APC targeting unit (SEQ IDs 36-43), human IgG3 hinge 1, hinge 4 and CH3 domain (SEQ ID NO:31, 32 and 34) and codon-optimized (for expression in mice) DNA encoding a peptide containing the sequentially coupled 5 neo-epitopes C22, C23, C25, C30, and C38 (SEQ ID NOs: 61-65) into the pUMVC4 expression cassette, see FIG. 5. As control, a pUMVC4 plasmid containing an insert without an APC targeting unit, however encoding human IgG3 hinge 1, hinge 4 and CH3 domain and the same 5 neoepitopes C22, C23, C25, C30, and C38 was used (SEQ ID NOs: 61-65). In the pUMVC4 APC targeting S16A vectors, the inserts also included a Kozak consensus sequence to effectively initiate translation. The 5 neoepitope amino acid sequences used in the experiments (also in the following examples) are set forth in the following table:

| Peptide | AA Sequence | SEQ ID NO: |
|---|---|---|
| C22 | QIETQQRKFKASRASILSEMKMLKEKR | SEQ ID NO: 61 |
| C23 | VILPQAPSGPSYATYLQPAQAQMLTPP | SEQ ID NO: 62 |
| C25 | DTLSAMSNPRAMQVLLQIQQGLQTLAT | SEQ ID NO: 63 |
| C30 | DGQLELLAQGALDNALSSMGALHALPR | SEQ ID NO: 64 |
| C38 | RLHVVKLLASALSTNAAALTQELLVLD | SEQ ID NO: 65 |

Plasmids pUMVC4 mCCL3 516A, pUMVC4 mCCL4 516A, pUMVC4 mCCL5 516A, pUMVC4 mCCL19 516A, pUMVC4 mXcl1 516A and (empty) pUMVC4 solubilised in sterile water were each mixed with poloxamer 188 (Kolliphor® from BASF) and Tyrode's buffer to obtain a composition of 3% w/v poloxamer 188 and 0.05 μg/μl plasmid in Tyrode's buffer.

The amphiphilic block co-polymers tested in combination with the DNA was Kolliphor® P188 (Or just referred to as Kolliphor in the present disclosure, or Lutrol® F 68), of the general formula:

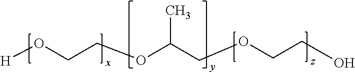

Study Plan

Mice received immunizations with the test vaccines on days −16, −9, −3, 5, and 12 relative to the CT26 tumour inoculation on day 0. Each immunization consisted of injection of 50 μl vaccine in the left and right tibia, respectively. Blood samples for C22 MHC I testing in a tetramer assay were obtained from the test animals on day 7 after inoculation.

6 groups of 14 mice received the following vaccine compositions respectively:

1. pUMVC4 mCCL3 516A 5 μg+Kolliphor

2. pUMVC4 mCCL4 516A 5 μg+Kolliphor

3. pUMVC4 mCCL5 516A 5 μg+Kolliphor

4. pUMVC4 mCCL19 516A 5 μg+Kolliphor

5. pUMVC4 mXCL1 516A 5 μg+Kolliphor

6. pUMVC4 5 μg+Kolliphor

7. Untreated control group

An eight group of naïve mice included 5 animals.

The tetramer assay was carried out as follows:

MHC class I molecules are produced and loaded with a stabilizing peptide that is exchanged with the C22 epitope by exposing the molecules to UV light. The MHC I molecules are multimerized by coupling to fluorescently labelled Streptavidin. To identify neo-peptide positive CD8+T cells, cells are co-stained with the multimers and fluorophore conjugated anti-CD3, anti-CD4 and anti-CD8 antibodies. Samples are then analyzed by flow cytometry and the fraction of MHC:C22 positive CD8+is calculated

To gauge T-cell activation, the following re-stimulation experiment was carried out:

Splenocytes were stimulated with the 5 vaccine-containing neo-peptides. In the splenocyte samples, antigen presenting cells process the neo-peptides and subsequently present them to T cells, leading to activation of cognate CD4+ and CD8+T cells. The activated T cells increase cytokine synthesis, including interferon γ (IFN-γ) and TNFα. IFN-γ as well as TNF\alpha producing T cells were detected by either ELISpot analysis og by flowcytometric analysis.

Results

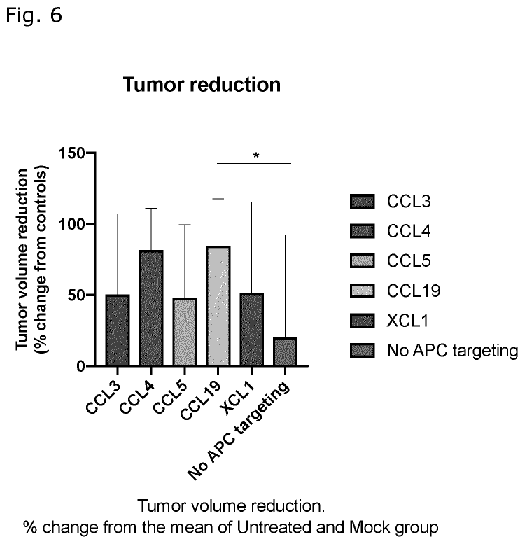

The effect on tumour growth of the immunizations is shown in FIG. 6: Prophylactic immunizations resulted in 50-100% lower tumour volume for mice receiving 5 μg pUMVC4 APC targeting S16A plasmid vector with co-polymers Kolliphor. The tumor reduction was significantly improved in the group treated with 5 μg pUMVC4 mCCL19 516A with Kolliphor as compared to 5 μg pUMVC4 with no APC targeting S16A with Kolliphor.

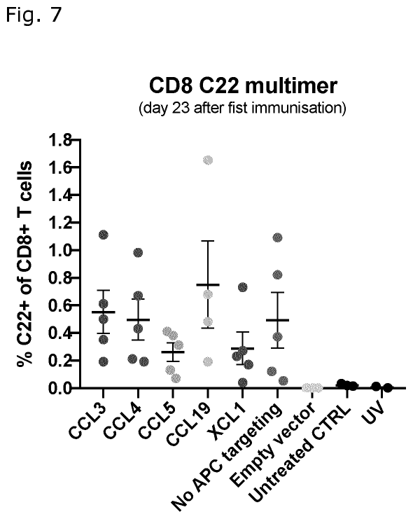

Whole blood from all mice where collected at day 7 post tumour inoculation and stained with fluorophore labelled C22 MHC I tetramers. Immunizations with the C22 encoding pUMVC4 APC targeting S16A vaccine induced C22 neo-peptide specific CD8+T cells at high frequencies (average from 0.3 to 0.6 frequency). See FIG. 7.

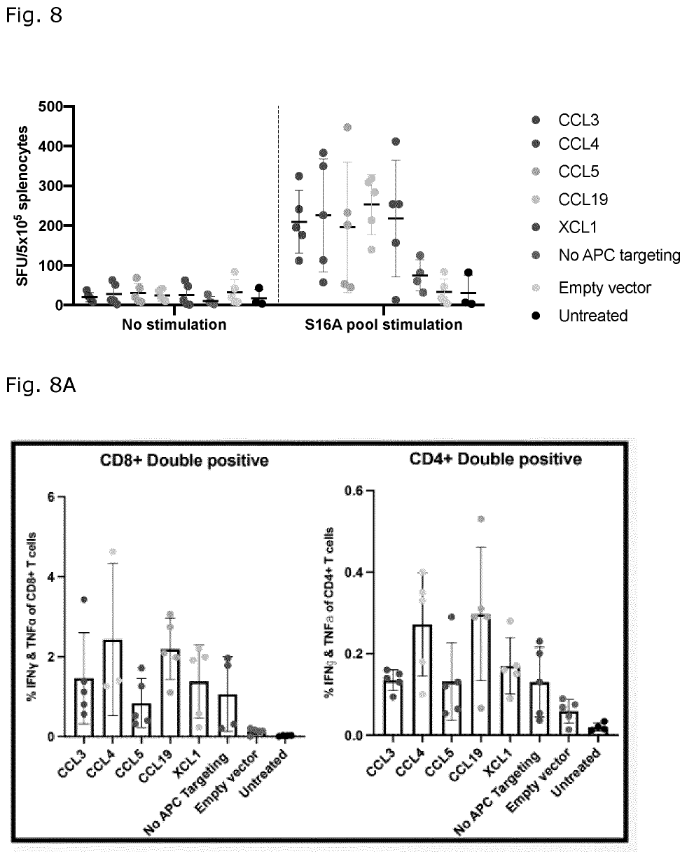

The vaccination with the S16A plasmid induced T cells capable of producing IFNγ in response to subsequent stimulation with neo-peptides, whereas samples from animals not immunized with S16A exhibited no cytokine signals upon stimulation with the neo-peptides. See FIG. 8.

Double-cytokine (INFγ and TNFα) producing CD8+andCD4+ T cells were observed in groups immunized with all constructs harbouring neoepitopes. Immunization with a version of the fusion protein not containing an APC targeting unit induced lover levels of double positive

CD4+T cells when compared to the targeted versions. Low or no signal was detected in negative control samples comparable to lower than background. See FIG. 8A.

Further, the DNA vaccines were well-tolerated by the mice; no signs of adverse effects were observed, and the body weight of the mice continuously increased throughout the study as evident from the increase of body weight change, indicative of healthy and unaffected mice.

Assessment of body weight change for mice immunized with DNA encoding
chemokines as APC targeting unit for delivery of neoepitopes

| | | |
|---|---|---|
| CCL3 | CCL4 | CCL5 |

| Days | mean | SD | N | mean | SD | N | mean | SD | N |
|---|---|---|---|---|---|---|---|---|---|
| −16 | 0 | 0 | 14 | 0 | 0 | 14 | 0 | 0 | 14 |
| −15 | 0.8863754 | 1.3309119 | 14 | 1.9423182 | 2.6624305 | 14 | 1.2735783 | 2.1555161 | 14 |
| −11 | 1.2574794 | 3.185451 | 14 | 2.1802261 | 3.3544355 | 14 | 4.3486595 | 4.0783625 | 14 |
| −9 | 0.220809 | 4.7964973 | 14 | 0.283442 | 3.0857483 | 14 | 3.0598174 | 4.661057 | 14 |
| −7 | 2.7427221 | 4.0704744 | 14 | 1.7739198 | 3.3477789 | 14 | 3.8801771 | 3.4525704 | 14 |
| −3 | 2.41419 | 4.4013287 | 14 | 2.5137031 | 4.8636941 | 14 | 4.9504809 | 3.8026727 | 14 |
| 1 | 2.3830701 | 4.762877 | 14 | 2.5370244 | 4.4049527 | 14 | 4.2645075 | 4.2478137 | 14 |
| 4 | 0.6451327 | 5.4944956 | 14 | 1.1731022 | 5.3330388 | 14 | 2.6162068 | 4.4934448 | 14 |
| 5 | 2.9150697 | 5.8397319 | 14 | 3.7419904 | 4.0857969 | 14 | 4.9224552 | 4.3104744 | 14 |
| 6 | 3.3658798 | 6.429654 | 14 | 4.5936267 | 3.8893079 | 14 | 5.112862 | 5.3888527 | 14 |
| 8 | 3.3451757 | 5.9851109 | 14 | 4.3506699 | 3.4117179 | 14 | 5.9742509 | 5.3270059 | 14 |
| 11 | 4.9910845 | 5.5779741 | 14 | 4.6701795 | 4.1931341 | 14 | 6.8725523 | 5.2499513 | 14 |
| 12 | 5.1640227 | 6.917586 | 14 | 4.4077461 | 3.7743819 | 14 | 7.702807 | 5.1709529 | 14 |
| 13 | 5.5916725 | 6.15574 | 14 | 5.1101704 | 5.1916002 | 14 | 8.3171774 | 5.4028654 | 14 |
| 15 | 6.2193684 | 7.3421511 | 14 | 6.1394359 | 4.7025327 | 14 | 8.5074718 | 4.6757277 | 14 |
| 18 | 7.2649069 | 7.775726 | 14 | 7.9329011 | 4.1813369 | 13 | 11.858912 | 5.5201394 | 12 |
| 20 | 11.524643 | 7.3146213 | 12 | 8.4188041 | 4.3620684 | 12 | 14.791602 | 5.3405828 | 12 |

| | | |
|---|---|---|
| CCL19 | XCL1 | No APC targeting |

| Days | mean | SD | N | mean | SD | N | mean | SD | N |
|---|---|---|---|---|---|---|---|---|---|
| −16 | 0 | 0 | 14 | 0 | 0 | 14 | 0 | 0 | 14 |
| −15 | 1.4415903 | 3.6817642 | 14 | −0.366308 | 3.2720866 | 14 | 0.8790025 | 1.2659416 | 14 |
| −11 | 1.4575305 | 3.8991463 | 14 | 0.9944677 | 4.1273903 | 14 | 1.957351 | 3.2190635 | 14 |
| −9 | 0.3365991 | 4.3996313 | 14 | 0.2157471 | 3.6052114 | 14 | 1.8717848 | 3.2461756 | 14 |
| −7 | 1.5629255 | 3.6565118 | 14 | 1.1870868 | 2.573715 | 14 | 2.1313298 | 3.1622751 | 14 |
| −3 | 2.0791244 | 4.6838491 | 14 | 2.6463785 | 4.2702206 | 14 | 3.9748831 | 2.3939841 | 14 |
| 1 | 3.1472585 | 6.6713694 | 14 | 3.2033645 | 4.0126726 | 14 | 4.4819436 | 3.1414049 | 14 |
| 4 | 0.8815458 | 6.3940253 | 14 | 1.3979628 | 6.0343284 | 14 | 2.3293464 | 4.337663 | 14 |
| 5 | 2.5304266 | 5.8941714 | 14 | 3.7315569 | 3.77523 | 14 | 4.4258209 | 4.1108208 | 14 |
| 6 | 2.4540823 | 4.7813688 | 14 | 4.8482379 | 3.8677259 | 14 | 4.7680391 | 4.4982046 | 14 |
| 8 | 2.7657444 | 4.6035696 | 14 | 4.43983 | 4.3553127 | 14 | 5.9081958 | 5.2074227 | 14 |
| 11 | 3.951975 | 5.2856939 | 14 | 6.5905758 | 5.3637017 | 14 | 5.0619503 | 4.2023491 | 14 |
| 12 | 5.1074123 | 5.2650377 | 14 | 5.7051446 | 5.4879028 | 14 | 6.3130952 | 3.9546609 | 14 |
| 13 | 5.188319 | 4.7721888 | 14 | 7.93995 | 5.7825594 | 14 | 7.8154602 | 4.4499296 | 14 |
| 15 | 6.266911 | 5.221704 | 14 | 7.8882129 | 5.9709272 | 14 | 8.9640968 | 4.814026 | 14 |
| 18 | 6.3197803 | 4.7643997 | 14 | 9.4112441 | 5.2223158 | 13 | 11.698208 | 3.0616748 | 12 |
| 20 | 7.7426458 | 4.7014022 | 13 | 12.864004 | 6.1921514 | 13 | 12.650733 | 4.3147454 | 11 |

| | Empty plasmid | | | Untreated | | | Naïve mice | | |
|---|---|---|---|---|---|---|---|---|---|
| Days | mean | SD | N | mean | SD | N | mean | SD | N |
| −16 | 0 | 0 | 14 | 0 | 0 | 13 | 0 | 0 | 5 |
| −15 | 1.3411582 | 1.6355957 | 14 | 1.6689002 | 3.3525154 | 13 | −0.118611 | 1.9190125 | 5 |
| −11 | 1.212813 | 2.731037 | 14 | 2.3310089 | 2.3660826 | 13 | −0.162533 | 1.9362329 | 5 |
| −9 | −0.257312 | 2.9411571 | 14 | 1.7733589 | 3.308048 | 13 | 1.3992846 | 3.9184503 | 5 |
| −7 | 0.634383 | 3.618878 | 14 | 2.0655841 | 4.0365386 | 13 | 2.3689432 | 3.7546783 | 5 |
| −3 | 3.0305458 | 4.5913096 | 14 | 3.444122 | 3.8061839 | 13 | 2.7756258 | 2.2299571 | 5 |
| 1 | 2.8390304 | 4.309384 | 14 | 3.5162211 | 4.2468904 | 13 | 3.0060962 | 1.076782 | 5 |
| 4 | 1.4497727 | 4.4667143 | 14 | −0.245309 | 7.7964932 | 13 | −0.249337 | 3.2368439 | 5 |
| 5 | 3.5042952 | 4.5394432 | 14 | 3.7433095 | 5.1731233 | 13 | 2.1795295 | 1.2681 | 5 |
| 6 | 3.7676028 | 3.7914055 | 14 | 4.422164 | 5.9939937 | 13 | 2.9423582 | 1.3548533 | 5 |
| 8 | 4.0142092 | 4.0396446 | 14 | 5.0562335 | 6.120553 | 13 | 3.4004358 | 1.1941085 | 5 |
| 11 | 6.27957 | 4.0380831 | 14 | 4.8483895 | 5.9276173 | 13 | 4.4244347 | 1.5398234 | 5 |
| 12 | 6.9780764 | 5.7678588 | 14 | 5.8418009 | 5.4677348 | 13 | 5.9894487 | 0.9256987 | 5 |
| 13 | 7.1979332 | 4.0540421 | 14 | 6.6638885 | 6.4712168 | 13 | 6.760866 | 1.752727 | 5 |
| 15 | 8.5884406 | 5.9671708 | 14 | 7.0848026 | 7.4386101 | 13 | 9.3695385 | 2.843451 | 5 |
| 18 | 10.358708 | 6.3822571 | 11 | 9.3929114 | 7.7285807 | 10 | 8.7600563 | 3.8239072 | 4 |
| 20 | 11.427638 | 5.4351893 | 9 | 8.7648269 | 8.578872 | 7 | 7.2360458 | 6.5963891 | 5 |

Conclusions

The Kolliphor delivered pUMVC4 plasmid vectors containing different APC targeting units and the S16A neoepitopes resulted in CT26 anti-tumour effects and circulating C22 neoepitope specific CD8+T cells. A dose as low as 5 μg of DNA resulted in highly significant tumour volume reduction compared to control groups, demonstrating the high efficacy of the APC targeting DNA vaccine. S16A neo-peptide re-stimulation showed similar T cell immunogenicity profiles in splenocytes across groups that received S16A Plasmid vector independent on the APC targeting unit.

The vaccines were well-tolerated by the mice; no signs of adverse effects were observed, and the body weight of the mice continuously increased throughout the study, indicative of healthy and unaffected mice.

### EXAMPLE 2

Assessment of other chemokines, cytokines, Fv fragments and peptides as APC targeting unit for delivery of neoepitopes

In this study we wished to test whether other chemokines and cytokines as well as APC targeting molecules outside the chemokine family, such as antibodies recognizing receptors on the APC or small peptide ligands binding to the surface receptors of the APC were able to induce neo-peptide specific T cells and reduce tumor growth to the same extend as seen in the previous study. Furthermore, we wished to monitor the impact of the vaccine on the well-being of vaccinated mice.

Plasmids for DNA vaccination were based on the commercially available pUMVC4™ vector available from Aldevron.

Eight expression vectors generated with the pUMVC4 as backbone and an APC targeting unit guiding the neoepitopes in S16A was constructed. The various chemokines, Fv fragments and peptide ligands used as APC targeting units are listed in the following table:

| APC targeting unit | SEQUENCE ID NO |
|---|---|
| mCCL19 | SEQ ID NO: 39 |
| mCCL20 | SEQ ID NO: 41 |
| mCCL21 | SEQ ID NO: 42 |
| mGM-CSF | SEQ ID NO: 43 |
| Anti-mDEC-205 Fv fragment | SEQ ID NO: 45 |
| Anti-mCLEC-9 Fv fragment | SEQ ID NO: 46 |
| CLEC9 ligand | SEQ ID NO: 47 |
| FLT3 ligand | SEQ ID NO: 48 |

Study plan

Mice received immunizations with the test vaccines on days −15, −8, −1, 6, and 13 relative to the CT26 tumour inoculation on day 0. Each immunization consisted of injection of 50 μl vaccine in the left and right tibia, respectively. Blood samples for C22 MHC I testing in a tetramer assay were obtained from the test animals on day −2 and 8 after inoculation.

9 groups of 13 mice received the following vaccine compositions respectively:
1. pUMVC4 mCCL19 516A 5 μg+Kolliphor
2. pUMVC4 (empty) 5 μg+Kolliphor
3. pUMVC4 anti mCLEC9 Fv S16A 5 μg+Kolliphor
4. pUMVC4 mCLEC-9 ligand S16A 5 μg+Kolliphor
5. pUMVC4 mCCL20 516A 5 μg+Kolliphor
6. pUMVC4 mCCL21 516A 5 μg+Kolliphor
7. pUMVC4 anti mDEC-205 Fv S16A 5 μg+Kolliphor
8. pUMVC4 mFLT3L S16A 5 μg+Kolliphor
9. pUMVC4 mGM-CSF S16A 5 μg+Kolliphor

A tenth group of naïve mice included 5 animals.

Read-outs of the experiment were tumour volume, measurement of neo-epitope-specific CD8+T cells in circulation and functional neo-epitope-specific T cells isolated from spleens.

Results

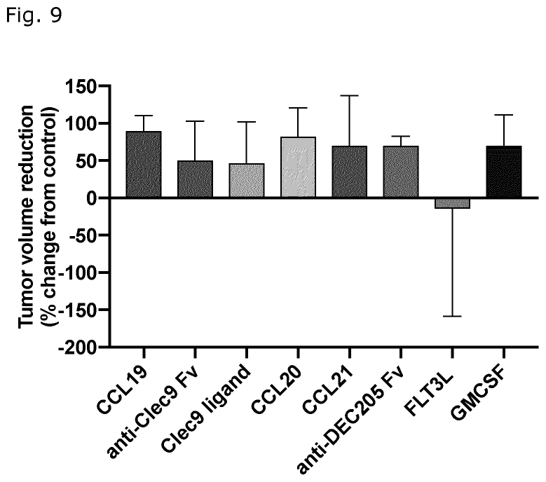

The effect on tumour growth of the immunizations is shown in FIG. 9: Prophylactic immunizations resulted in 50-100% tumor size reduction for mice receiving 5 μg pUMVC4 APC targeting S16A plasmid vector with co-polymers Kolliphor compared to 5 μg pUMVC4 with Kolliphor.

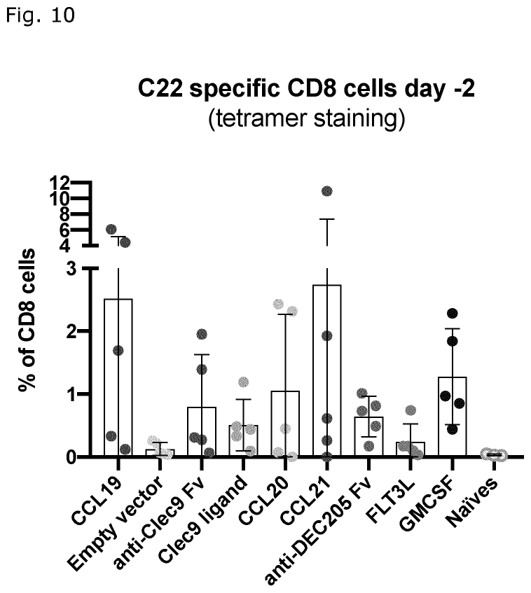

Whole blood from all mice where collected at day −2 and stained with fluorophore labelled C22 MHC I tetramers. Immunizations with the C22 encoding pUMVC4 APC targeting S16A vaccine induced C22 neo-peptide specific CD8+T cells at high frequencies (average from 0.3 to 0.6 frequency). See FIG. 10. The effect was markedly better than the control empty vector (average under 0.1 frequency) without neoepitopes.

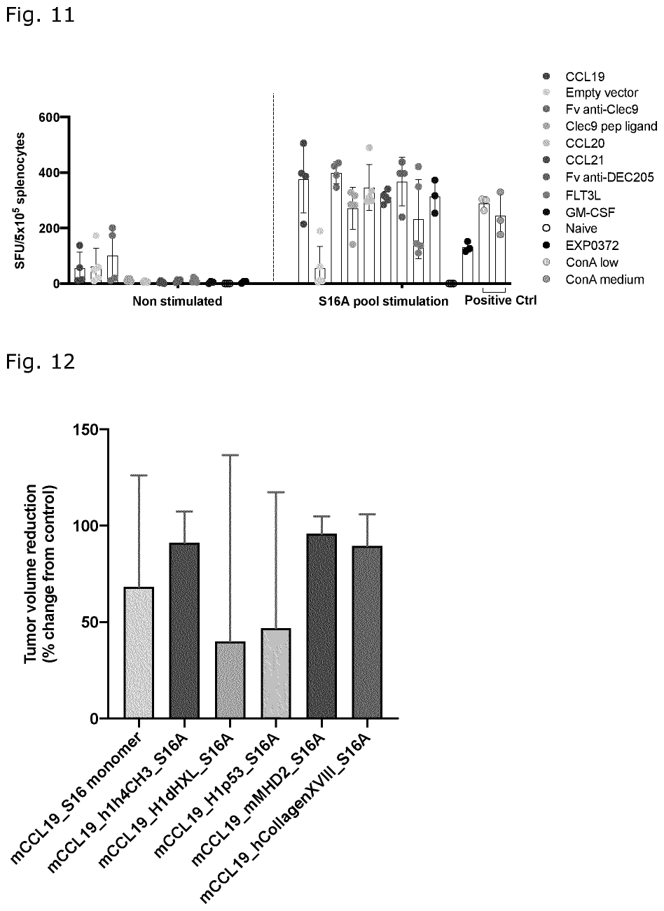

At endpoint vaccination with all the S16A containing plasmids induced T cells derived from spleen that were capable of producing IFNγ in response to subsequent stimulation with neo-peptides. Samples from animals that were not immunized with S16A (empty plasmid and naïve mice) exhibited no cytokine signals upon stimulation with the neo-peptides. See FIG. 11.

Further, the DNA vaccines were well-tolerated by the mice; no signs of adverse effects were observed, and the body weight of the mice continuously increased throughout the study, as evident from the increase on body weight change, indicative of healthy and unaffected mice.

Assessment of body weight change for mice immunized with DNA encoding Fv
fragments and peptides as APC targeting unit for delivery of neoepitopes

| | CCL19 | | | Empty vector | | | Fv anti-Clec9 | | | Clec9 pep ligand | | |
|---|---|---|---|---|---|---|---|---|---|---|---|---|
| Days | mean | SD | N | mean | SD | N | mean | SD | N | mean | SD | N |
| −15 | 0 | 0 | 13 | 0 | 0 | 8 | 0 | 0 | 13 | 0 | 0 | 13 |
| −14 | 1.139148 | 1.353041 | 13 | 0.7274891 | 1.6277915 | 8 | 1.0104646 | 2.2013327 | 13 | 1.4586595 | 1.9248236 | 13 |
| −12 | 1.9608584 | 2.8363333 | 13 | 0.076736 | 2.0535941 | 8 | 0.7951009 | 2.8320476 | 13 | 2.1721557 | 2.9426216 | 13 |
| −8 | 4.8871238 | 3.3673985 | 13 | 2.9945308 | 3.5834629 | 8 | 2.4139883 | 4.3354556 | 13 | 4.27686 | 4.2014545 | 13 |
| −5 | 3.993838 | 2.417825 | 13 | 3.3228029 | 2.966784 | 8 | 6.0817445 | 3.1310146 | 13 | 5.012723 | 3.5305563 | 13 |
| −1 | 2.5248411 | 1.7932877 | 13 | 4.8718416 | 2.8314056 | 8 | 4.2528719 | 3.0745385 | 13 | 4.7154367 | 2.5850021 | 13 |
| 2 | 6.0475665 | 2.4763482 | 13 | 7.2299469 | 1.5077816 | 8 | 6.5465987 | 3.2364261 | 13 | 7.6309345 | 2.5155128 | 13 |
| 5 | 7.4167472 | 2.8104202 | 13 | 8.1439476 | 2.9839372 | 8 | 7.8874323 | 3.2500325 | 13 | 7.8902886 | 2.9573388 | 13 |
| 7 | 8.0583294 | 3.2876568 | 13 | 10.222268 | 3.6128255 | 8 | 9.0977444 | 3.9930344 | 13 | 8.3282824 | 2.6349014 | 13 |
| 9 | 7.8405825 | 3.8765573 | 13 | 9.5793539 | 3.052331 | 8 | 8.9420401 | 3.6235839 | 13 | 8.8672662 | 2.9374285 | 13 |
| 12 | 9.3909254 | 2.8600445 | 13 | 10.344554 | 4.3233427 | 8 | 10.068073 | 5.2140401 | 13 | 10.69861 | 3.2917778 | 13 |
| 14 | 8.0597907 | 2.7665782 | 13 | 10.881072 | 3.9925636 | 8 | 9.8156458 | 3.7635493 | 13 | 9.818513 | 2.8993949 | 13 |
| 16 | 9.6136928 | 2.6133819 | 13 | 14.541798 | 5.6578245 | 8 | 12.050101 | 2.9271745 | 12 | 11.801434 | 3.223206 | 13 |
| 19 | 10.352757 | 3.5465027 | 12 | 15.176842 | 5.9073788 | 7 | 13.363635 | 4.0756288 | 11 | 11.143421 | 3.7802431 | 13 |

| | CCL20 | | | CCL21 | | | Fv anti-DEC205 | | |
|---|---|---|---|---|---|---|---|---|---|
| Days | mean | SD | N | mean | SD | N | mean | SD | N |
| −15 | 0 | 0 | 13 | 0 | 0 | 13 | 0 | 0 | 13 |
| −14 | 0.6307569 | 2.4728171 | 13 | 1.3543557 | 1.7192062 | 13 | 0.6293231 | 1.9084697 | 13 |
| −12 | 2.1195786 | 3.2126495 | 13 | 1.8293204 | 1.8917054 | 13 | 2.100173 | 3.1837618 | 13 |
| −8 | 4.6943438 | 2.9687584 | 13 | 4.3742356 | 2.9070852 | 13 | 4.1636965 | 2.5322817 | 13 |
| −5 | 4.3350389 | 1.9419675 | 13 | 5.5359249 | 2.966961 | 13 | 4.5458723 | 2.2127792 | 13 |
| −1 | 3.9570983 | 1.9996787 | 13 | 4.3963027 | 2.8908727 | 13 | 3.8951226 | 2.6855639 | 13 |
| 2 | 7.8824803 | 3.9559045 | 13 | 5.3131697 | 2.8459311 | 13 | 6.5721784 | 3.5730706 | 13 |
| 5 | 8.7628661 | 3.7619537 | 13 | 7.4036597 | 5.122963 | 13 | 7.1031392 | 3.9039116 | 13 |
| 7 | 11.224134 | 1.9098859 | 13 | 9.7279721 | 3.3447985 | 13 | 9.7004289 | 3.8788376 | 13 |
| 9 | 9.7266611 | 3.6525425 | 13 | 9.4743827 | 3.7748095 | 13 | 8.7800297 | 5.5312064 | 13 |
| 12 | 11.340991 | 2.9263815 | 13 | 9.7580625 | 2.6018417 | 13 | 11.043509 | 3.6359454 | 13 |
| 14 | 9.742928 | 3.7752266 | 13 | 9.6435734 | 2.4700711 | 13 | 10.085142 | 3.0395913 | 13 |
| 16 | 11.899696 | 3.4063403 | 13 | 11.569268 | 3.3297408 | 13 | 11.596663 | 2.8781698 | 12 |
| 19 | 12.246312 | 3.8715511 | 13 | 11.66981 | 3.477578 | 13 | 11.882038 | 2.5270066 | 12 |

| | FLT3L | | | GM-CSF | | | Naïve | | |
|---|---|---|---|---|---|---|---|---|---|
| Days | mean | SD | N | mean | SD | N | mean | SD | N |
| −15 | 0 | 0 | 13 | 0 | 0 | 13 | 0 | 0 | 4 |
| −14 | 1.6487397 | 3.0170806 | 13 | 1.5531263 | 1.7891121 | 13 | −0.563251 | 0.8442212 | 4 |
| −12 | 3.1594828 | 3.6284443 | 13 | 3.0322935 | 1.4896749 | 13 | −0.803813 | 1.7905142 | 4 |
| −8 | 6.4052386 | 5.152845 | 13 | 5.1544162 | 4.6237373 | 13 | 3.3374585 | 2.5580524 | 4 |
| −5 | 6.4713217 | 4.1792643 | 13 | 8.29216 | 3.3715389 | 13 | 6.0071692 | 4.7847333 | 4 |
| −1 | 4.9651132 | 2.2349646 | 12 | 5.8838183 | 2.5453088 | 13 | 3.096435 | 1.3922387 | 4 |
| 2 | 7.8271327 | 3.7384128 | 12 | 7.6394922 | 3.0395695 | 13 | 5.967283 | 1.5412159 | 4 |
| 5 | 8.8922992 | 3.8481706 | 12 | 8.6807205 | 3.2160132 | 12 | 6.4137115 | 1.5640278 | 4 |
| 7 | 9.8318543 | 4.8462709 | 12 | 10.390035 | 3.8090331 | 12 | 7.6586037 | 2.8712901 | 4 |
| 9 | 9.5842145 | 3.5666071 | 12 | 9.9943916 | 3.9615974 | 12 | 8.6974545 | 0.9864127 | 4 |
| 12 | 11.190934 | 4.0935224 | 12 | 12.309059 | 4.5705428 | 12 | 9.522338 | 2.2707586 | 4 |
| 14 | 11.369933 | 3.6257843 | 12 | 10.605977 | 3.3638856 | 12 | 10.327501 | 2.7798875 | 4 |
| 16 | 14.019179 | 5.0670855 | 12 | 13.42857 | 3.6152313 | 12 | 11.444177 | 2.2889767 | 4 |
| 19 | 13.274705 | 2.505272 | 10 | 13.055231 | 3.2021551 | 12 | 9.7444706 | 2.2332287 | 4 |

Conclusions

The Kolliphor delivered pUMVC4 plasmid vectors containing different APC targeting units and the S16A neoepitopes resulted in CT26 anti-tumour effects and circulating C22 neoepitope specific CD8+T cells. A dose as low as 5 μg of DNA resulted in tumour volume reduction compared to control groups, demonstrating the high efficacy of the APC targeting DNA vaccine. S16A neo-peptide re-stimulation showed similar T cell immunogenicity profiles in splenocytes across groups that received S16A Plasmid vector independent on the APC targeting unit. Furthermore, the DNA vaccines were well-tolerated by the mice as assessed by body weight change during the experiment.

### EXAMPLE 3

Assessment of various multimerization units for the APC targeting technology for delivery of neoepitopes

In this study we wished to test whether an APC targeting neoepitope vaccine with multimerization units other than a fragment from human IgG3, such as other immuno globulins (Ig), synthetic proteins or collagen fragments, were able to induce neo-peptide specific T cells and reduce tumor growth. Murine CCL19 was used as APC targeting unit. Furthermore, we wished to monitor the impact of the vaccine on the well-being of vaccinated mice

Plasmids for DNA vaccination were based on the commercially available pUMVC4™ vector available from Aldevron.

Five new expression vectors generated with the pUMVC4 as backbone and CCL19 as APC targeting unit guiding the neoepitopes in S16A were constructed. The various multimerization units used for the various constructs are listed in the table below:

| Molecule | Function | SEQ ID NO: |
|---|---|---|
| GS linker (for monomer) | No multimerization | SEQ ID NO: 13 |
| dHLX protein | dimerization | SEQ ID NO: 49 |
| hMHD2 (human IgM) | dimerization | SEQ ID NO: 51 |
| Collagen XVIII trimerisation | Trimerization | SEQ ID NO: 52 |
| domain | | |
| p53 synthetic protein | Tetramerization | SEQ ID NO: 53 |

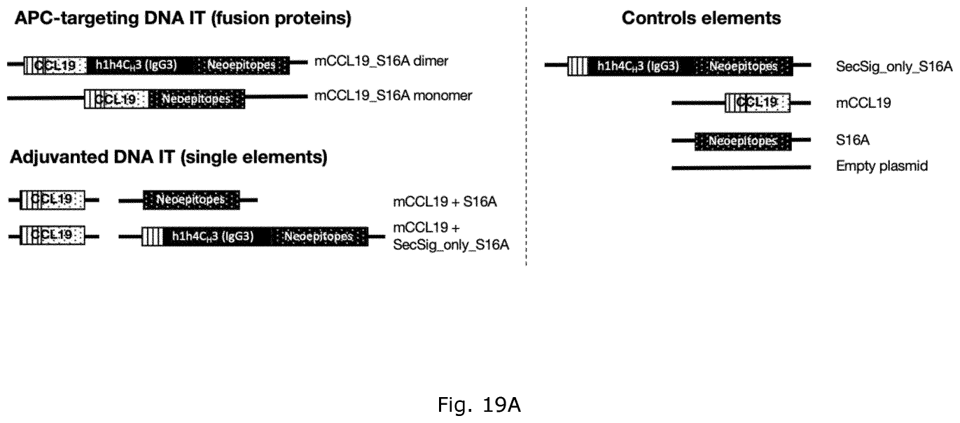

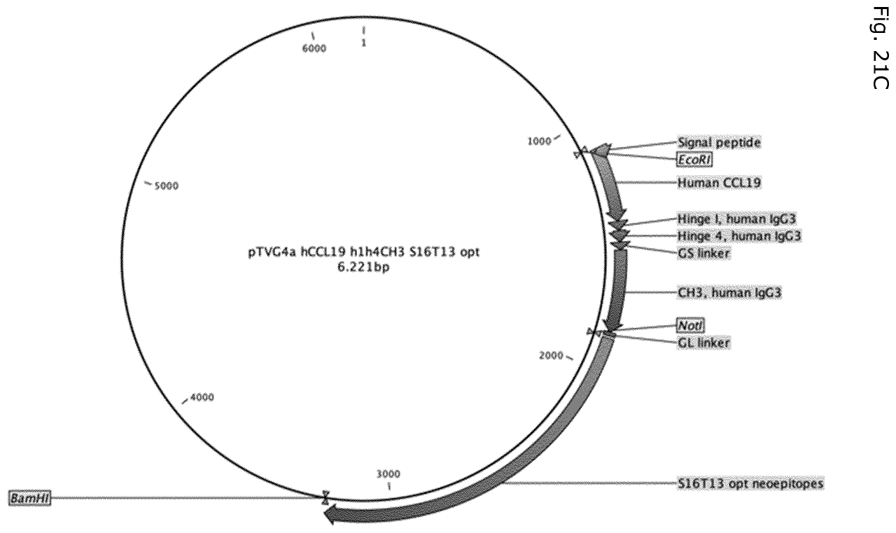

The new DNA constructs were compared to mCCL19 H1H4CH3 516A construct with IgG3 dimerization domain (SEQ ID NO: 59, 60, and 34). See overview in FIGS. 18 and 20.

Study plan

Mice received immunizations with the test vaccines on days -14, −7, 1, 8, and 15 relative to the CT26 tumour inoculation on day 0. Each immunization consisted of injection of 50 μl vaccine in the left and right tibialis anterior, respectively. Blood samples for C22 MHC I testing in a tetramer assay were obtained from the test animals on day 7 after inoculation.

7 groups of 13 mice received the following vaccine compositions respectively:
1. pUMVC4 (empty) 5 μg+Kolliphor
2. pUMVC4 mCCL19 516A monomer 5 μg+Kolliphor
3. pUMVC4 mCCL19 H1H4CH3 516A 5 μg+Kolliphor
4. pUMVC4 mCCL19 H1dHLX S16A 5 μg+Kolliphor
5. pUMVC4 mCCL19 H1p53 516A 5 μg+Kolliphor
6. pUMVC4 mCCL19 hMHD2 516A 5 μg+Kolliphor
7. pUMVC4 mCCL19 mCollagenXVIII S16A 5 μg+Kolliphor

An eighth group of naïve mice included 4 animals.

Read-outs of the experiment were body weight change relative to the weight at the first immunization, tumour volume reduction, measurement of neo-epitope-specific CD8+T cells in circulation and functional neo-epitope-specific T cells isolated from spleens.

Results

The effect on tumour growth of the immunizations is shown in FIG. 12: Prophylactic immunizations resulted in 50-100% tumor size reduction for all groups of mice receiving 5 μg plasmid vector pUMVC4 mCCL19 containing the neoepitopes S16A in combination with Kolliphor compared to 5 μg empty plasmid with Kolliphor. The groups receiving a DNA design with a multimerization unit that was of Ig origin or collagen performed the best.

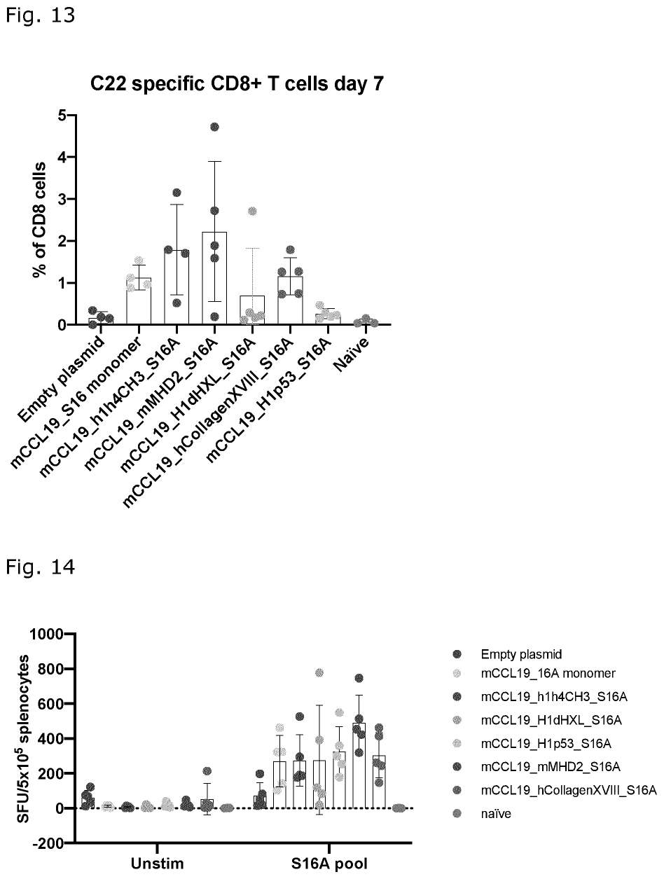

Whole blood from all mice where collected at day 7 and stained with fluorophore labelled C22 MHC I tetramers. Immunizations with the C22 encoding pUMVC4 CCL19 S16A vaccine all gave rise to a detectable level of C22 neo-peptide specific CD8+T cells (average ranging from 0.3 to 5% frequency). See FIG. 13. The effect was most pronounced for the IgG containing designs (H1H4CH3 and hMHD2, average around 2% frequency), medium response for the monomer and the trimer (monomer and hCollagenXVIII, average over 1% frequency), which were markedly better than the synthetic protein designs (H1dHLX and H1p53, average below 0.5% frequency) and the empty plasmid control.

At endpoint vaccination with the all the S16A containing plasmids induced T cells derived from spleen that were capable of producing IFNγ in response to subsequent stimulation with neo-peptides. Samples from animals that were not immunized with S16A (empty plasmid and naïve) exhibited no cytokine signals upon stimulation with the neo-peptides. See FIG. 14.

Further, the DNA vaccines were well-tolerated by the mice; no signs of adverse effects were observed, and the body weight of the mice continuously increased throughout the study, as evident from the increase in body weight change, indicative of healthy and unaffected mice.

Assessment of body weight change for mice immunized with DNA encoding various multimerization
units for the APC targeting technology for delivery of neoepitopes

| | Empty plasmid | | | mCCL19_S16 monomer | | | mCCL19_h1h4CH3_S16A | | |
|---|---|---|---|---|---|---|---|---|---|
| Days | mean | SD | N | mean | SD | N | mean | SD | N |
| −14 | 0 | 0 | 15 | 0 | 0 | 14 | 0 | 0 | 15 |
| −13 | 1.8065903 | 1.4658816 | 15 | 1.6868882 | 2.0734565 | 14 | 2.051628 | 1.9325912 | 15 |
| −10 | 3.5160765 | 2.1254483 | 15 | 4.6467974 | 2.9015154 | 14 | 4.6245979 | 2.0965962 | 15 |
| −7 | 3.770133 | 2.0686022 | 15 | 4.8105898 | 4.0003457 | 14 | 5.4134208 | 2.8454668 | 15 |
| −5 | 6.628435 | 3.010984 | 15 | 7.1382895 | 4.7086045 | 14 | 9.0138749 | 3.3928789 | 15 |
| −3 | 7.0289117 | 2.4257649 | 15 | 7.0974051 | 4.3724483 | 14 | 9.1890066 | 3.1346121 | 15 |
| 0 | 7.3468783 | 3.4857441 | 15 | 8.3409671 | 3.9141863 | 14 | 8.4347524 | 2.1744504 | 15 |
| 1 | 4.5328145 | 3.6591673 | 15 | 5.4788081 | 4.116406 | 14 | 6.6809992 | 3.0757316 | 15 |
| 2 | 5.9201369 | 3.5739631 | 15 | 8.0948364 | 5.1165168 | 14 | 8.8516984 | 3.5081711 | 15 |
| 4 | 6.85005 | 3.4916068 | 15 | 9.0792098 | 3.9091538 | 14 | 10.529619 | 3.8993072 | 15 |
| 7 | 7.1445987 | 2.9992626 | 15 | 11.510528 | 6.0430493 | 14 | 11.210636 | 4.2788492 | 15 |
| 9 | 10.542385 | 4.6710614 | 15 | 10.840238 | 3.6489439 | 14 | 13.746003 | 5.5857997 | 15 |
| 11 | 10.462602 | 3.9313322 | 15 | 12.058571 | 2.3601854 | 14 | 14.688285 | 6.4618683 | 15 |
| 14 | 13.315998 | 4.5686599 | 15 | 13.551067 | 3.7850659 | 14 | 14.282771 | 5.9143999 | 15 |
| 16 | 13.755865 | 4.6056887 | 14 | 14.472833 | 5.4109189 | 13 | 15.354434 | 5.5457766 | 15 |
| 18 | 15.261076 | 3.7202453 | 14 | 16.056548 | 5.4333788 | 12 | 17.775144 | 7.3666753 | 14 |

| | mCCL19_H1dHXL_S16A | | | mCCL19_H1p53_S16A | | | mCCL19_mMHD2_S16A | | |
|---|---|---|---|---|---|---|---|---|---|
| Days | mean | SD | N | mean | SD | N | mean | SD | N |
| −14 | 0 | 0 | 14 | 0 | 0 | 14 | 0 | 0 | 14 |
| −13 | 2.5402176 | 1.3812768 | 14 | 1.7149315 | 2.2405615 | 14 | 1.0994299 | 1.7883377 | 14 |
| −10 | 4.0723021 | 2.1302442 | 14 | 5.21012 | 3.4689379 | 14 | 3.0213857 | 2.5928036 | 14 |
| −7 | 4.9851396 | 1.9122281 | 14 | 4.7338799 | 4.3510254 | 14 | 4.2950687 | 2.9643819 | 14 |
| −5 | 7.5185625 | 3.0997695 | 14 | 6.0298098 | 4.0751979 | 14 | 6.7843427 | 2.9550719 | 14 |
| −3 | 7.619037 | 3.197423 | 14 | 6.3863026 | 3.5903038 | 14 | 7.020561 | 3.1511815 | 14 |
| 0 | 6.5620459 | 2.8396834 | 14 | 5.0115533 | 3.4082094 | 14 | 4.0979031 | 5.4476714 | 14 |
| 1 | 5.0012801 | 3.6445776 | 14 | 4.1558597 | 3.3628039 | 14 | 3.2625642 | 3.8224016 | 14 |
| 2 | 4.8054743 | 3.5353776 | 14 | 4.6349486 | 3.6688398 | 14 | 4.0306469 | 2.8672514 | 14 |
| 4 | 6.6966493 | 4.0431882 | 14 | 6.5396079 | 3.8835809 | 14 | 6.1143302 | 2.917494 | 14 |
| 7 | 7.6657322 | 4.5257821 | 14 | 8.2979131 | 3.4064541 | 14 | 7.2981185 | 3.7648275 | 14 |
| 9 | 10.329819 | 4.839032 | 14 | 10.093287 | 3.6543979 | 14 | 11.022503 | 4.1735587 | 14 |
| 11 | 9.9963472 | 3.7006016 | 14 | 11.298379 | 4.1873395 | 14 | 11.46006 | 5.5782851 | 14 |
| 14 | 10.760224 | 3.9935569 | 13 | 12.696664 | 3.7677507 | 14 | 12.026869 | 5.2600742 | 14 |
| 16 | 13.055214 | 3.9912675 | 13 | 13.333518 | 4.8120939 | 14 | 12.579142 | 4.4309364 | 13 |
| 18 | 14.707669 | 5.3675715 | 12 | 14.617345 | 3.6781525 | 13 | 14.078862 | 5.3951134 | 11 |

| | mCCL19_hCollagenXVIII_S16A | | | Naïve | | |
|---|---|---|---|---|---|---|
| Days | mean | SD | N | mean | SD | N |
| −14 | 0 | 0 | 14 | 0 | 0 | 4 |
| −13 | 1.638233 | 1.4133702 | 14 | 1.0119102 | 0.9299167 | 4 |
| −10 | 4.1856776 | 2.6809016 | 14 | 5.3438235 | 1.4548287 | 4 |
| −7 | 4.4349953 | 2.7134899 | 14 | 5.150205 | 3.0529469 | 4 |
| −5 | 6.7596373 | 2.4500964 | 14 | 6.7241385 | 2.8091057 | 4 |
| −3 | 7.2101461 | 3.3531306 | 14 | 6.6671358 | 3.0599535 | 4 |
| 0 | 8.1756837 | 4.4501707 | 14 | 6.8818255 | 3.1676591 | 4 |
| 1 | 5.5607829 | 4.9334063 | 14 | 3.4020269 | 2.5567755 | 4 |
| 2 | 7.211852 | 4.228828 | 14 | 5.0527072 | 3.859303 | 4 |
| 4 | 8.3746865 | 5.0902167 | 14 | 7.3629684 | 2.1437738 | 4 |
| 7 | 8.7555461 | 4.1548737 | 14 | 8.0605716 | 3.2966171 | 4 |
| 9 | 10.427133 | 4.2394149 | 14 | 10.407827 | 3.1963835 | 4 |
| 11 | 11.462716 | 3.88127 | 14 | 11.031481 | 3.5252434 | 4 |
| 14 | 11.312763 | 3.9910123 | 14 | 11.057459 | 5.3188489 | 4 |
| 16 | 11.480441 | 4.5051312 | 11 | 12.035978 | 4.2855628 | 4 |
| 18 | 13.006091 | 3.3628816 | 11 | 16.391946 | 5.8096504 | 3 |

Conclusions

The Kolliphor delivered pUMVC4 plasmid vectors containing mCCL19, different multimerization units and the S16A neoepitopes resulted in CT26 anti-tumour effects from 50-100%. The groups receiving a DNA design with a multimerization unit that was of Ig origin or collagen performed the best. The neoepitope specific CD8+ T cell response at day 7 corresponded to the antitumor effects and the level of functional T cells at endpoint (as measured by IFNγ secretion) was comparable between groups receiving a DNA design containing neoepitopes. Furthermore, the DNA vaccines were well-tolerated by the mice as assessed by body weight change during the experiment.

### EXAMPLE 4

Assessment of combination/dissociation of the separate units for the APC targeting technology for delivery of neoepitopes

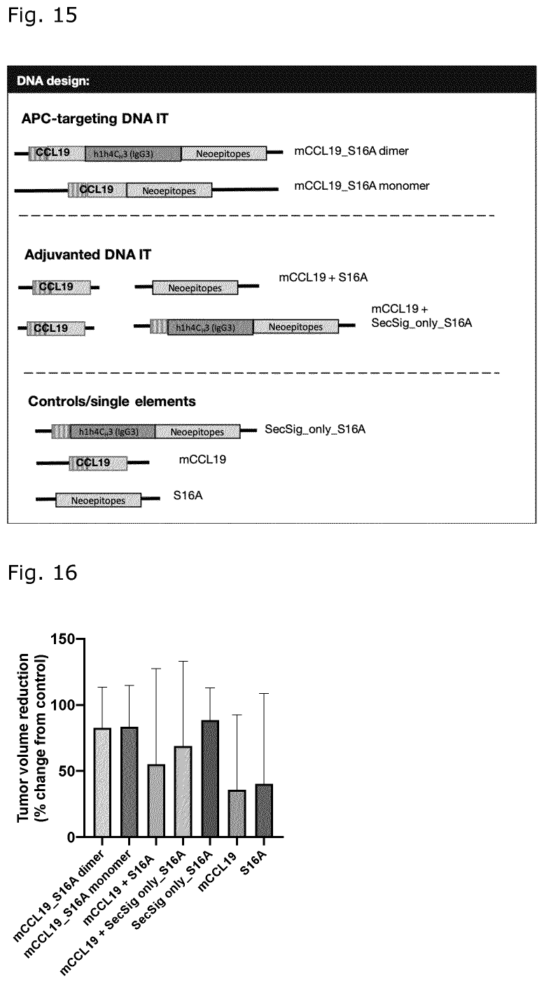

In this study we wished to test whether all three modules; APC targeting, multimerization domain and neoepitopes, needed to be physically linked together as a fusion protein product to perform optimally, or if the mere presence of the chemokine as an adjuvant is sufficient as well as if the dimer/secretion of the neoepitopes ads to the anti-tumor effect and T cell response for the APC targeting neoepitope vaccine. Murine CCL19 was used as chemokine for all DNA constructs. See overview in FIG. 15.

Plasmids for DNA vaccination were based on the commercially available pUMVC4™ vector available from Aldevron.

Three new expression vectors were generated with the pUMVC4 as backbone containing either mCCL19 alone (SEQ ID NO: 39), the neoepitopes S16A alone or the secretion signal (SecSig), the dimerization domain H1H3CH3 of IgG3 (SEQ ID NO: 59, 60 and 34) and S16A neoepitopes. Listed in FIG. 15.

The new DNA constructs, and combinations hereof, were compared to mCCL19 H1H4CH3 516A construct encoding the fusion protein (referred to as mCCL19 516A dimer below) as well as the mCCL19 516A monomer.

Study plan

Mice received immunizations with the test vaccines on days -14, −7, 1, 8, and 15 relative to the CT26 tumour inoculation on day 0. Each immunization consisted of injection of 50 μl vaccine in the left and right tibialis anterior, respectively. Blood samples for C22 MHC I testing in a tetramer assay were obtained from the test animals on day 9 after inoculation.

8 groups of 13 mice received the following vaccine compositions respectively:
1. pUMVC4 (empty) 10 μg+Kolliphor
2. pUMVC4 mCCL19 516A dimer 5 μg+Kolliphor
3. pUMVC4 mCCL19 516A monomer
4. pUMVC4 mCCL19 5 μg+pUMVC4 516A 5 μg+Kolliphor
5. pUMVC4 mCCL19 5 μg+pUMVC4 SecSig H1H4CH3 516A 5 μg+Kolliphor
6. pUMVC4 SecSig H1H4CH3 516A 5 μg+Kolliphor
7. pUMVC4 mCCL19 5 μg+Kolliphor
8. pUMVC4 516A 5 μg+Kolliphor

A ninth group of naïve mice included 4 animals.

Read-outs of the experiment were body weight change relative to the weight at the first immunization, tumour volume, and measurement of neo-epitope-specific CD8+T cells in circulation. Also, a re-stimulation experiment was carried out assessing the presence of CD8+ and CD4+ cells producing IFNγ and TNFα.

Results

The effect on tumour growth of the immunizations is shown in FIG. 16: Prophylactic immunizations resulted in tumour size reduction for all groups of mice receiving mCCL19 and neoepitopes, independent of if the components are encoded as a fusion protein or as separate protein products. The non-targeted fusion protein comprised of SecSig, H1H4CH3 dimerization domain and neoepitopes also had significant antitumor effect, indicating that secretion and dimerization of the neoepitopes improves the antitumor effect.

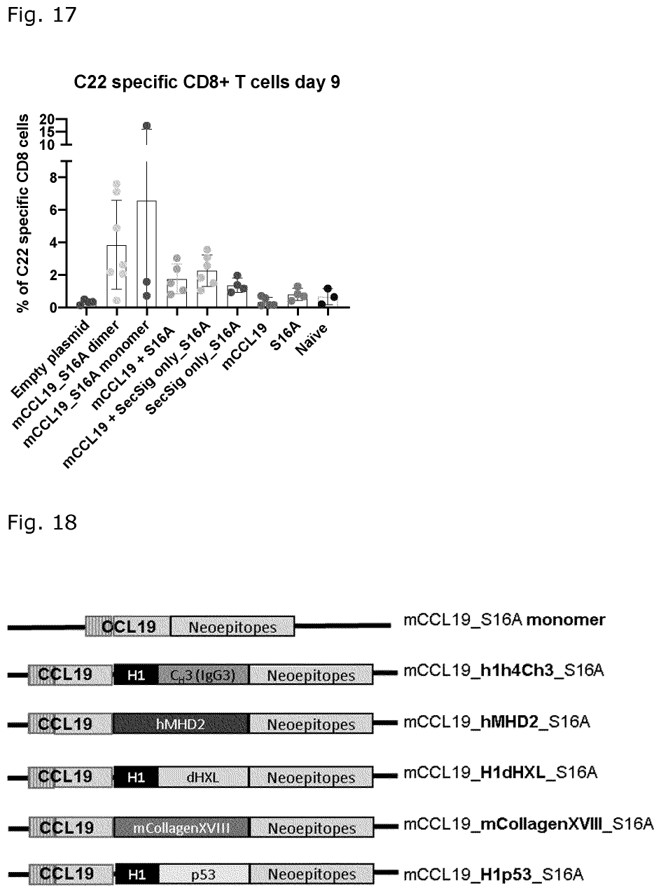

Whole blood from all mice where collected at day 9 and stained with fluorophore labelled C22 MHC I tetramers. Immunizations with all vaccines containing the epitope C22, which is part of S16A, gave rise to a detectable level of C22 neo-peptide specific CD8+T cells (average ranging from 0.5 to 15% frequency). See FIG. 17. The effect was most pronounced for the fusion protein designs (mCCL19 516A dimer and mCCL19 516A monomer, average around 4% frequency), whereas the combination of the individual mCCL19 product with the non-targeted fusion protein comprised of SecSig, H1H4CH3 dimerization domain and neoepitopes also gave rise to high T cell levels (average over 1.5% frequency), which were markedly better than the effect of the single components (mCCL19 and S16A, average below 0.5% frequency) and the empty plasmid control.

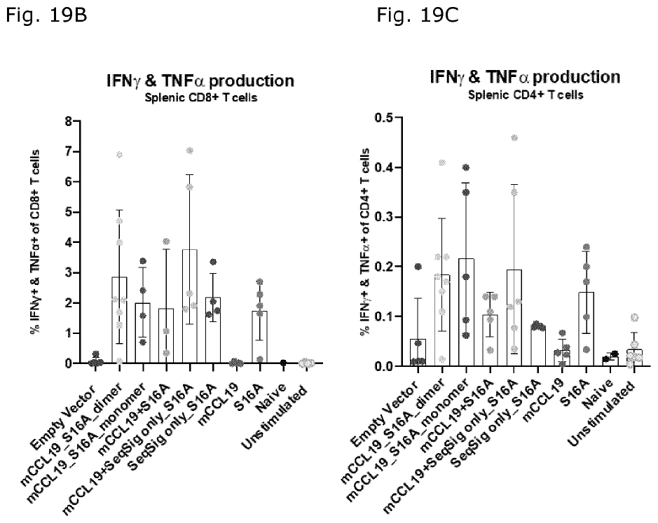

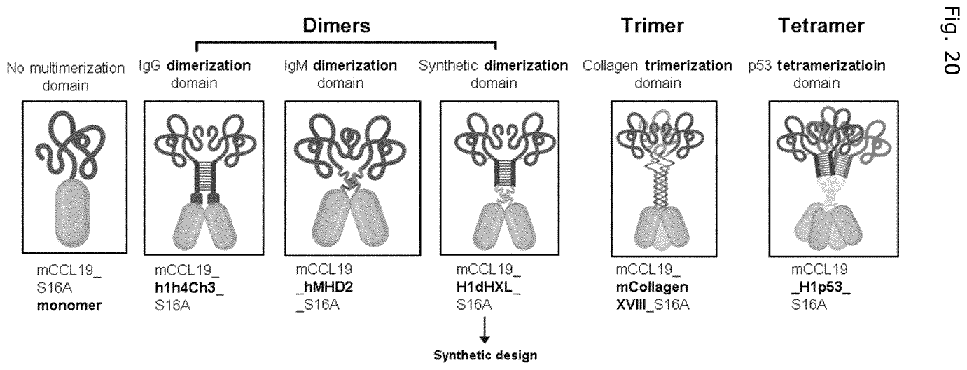

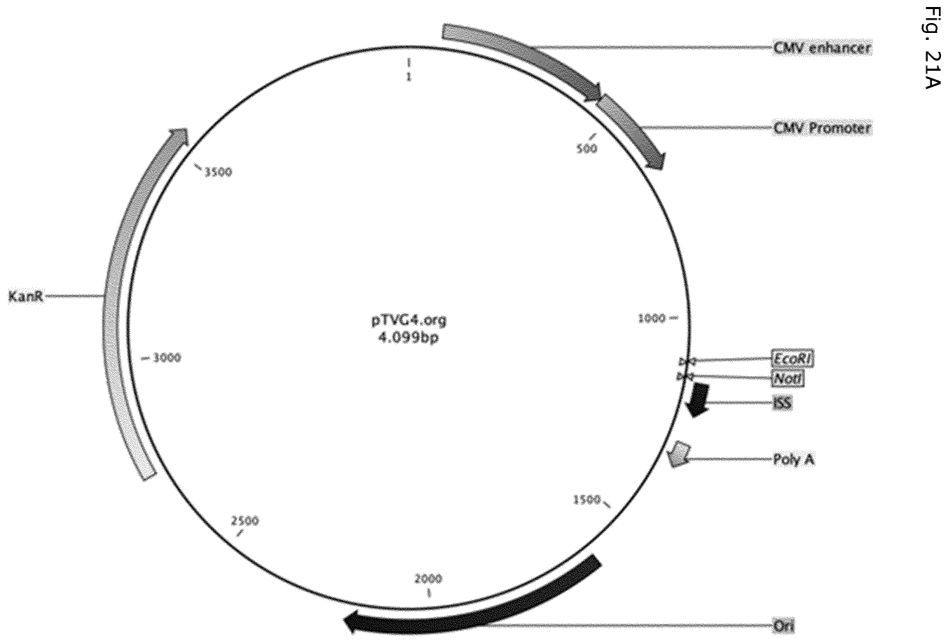

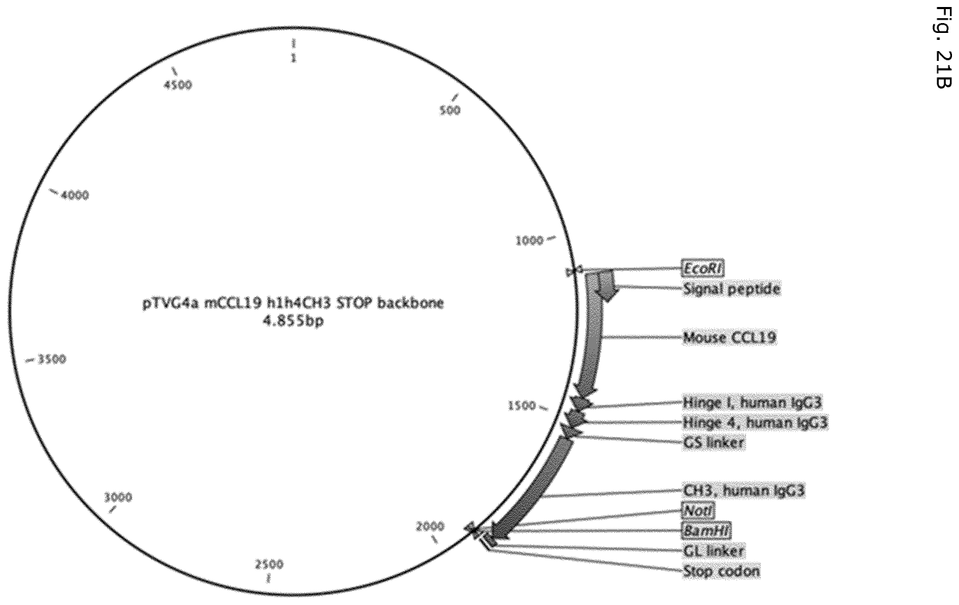

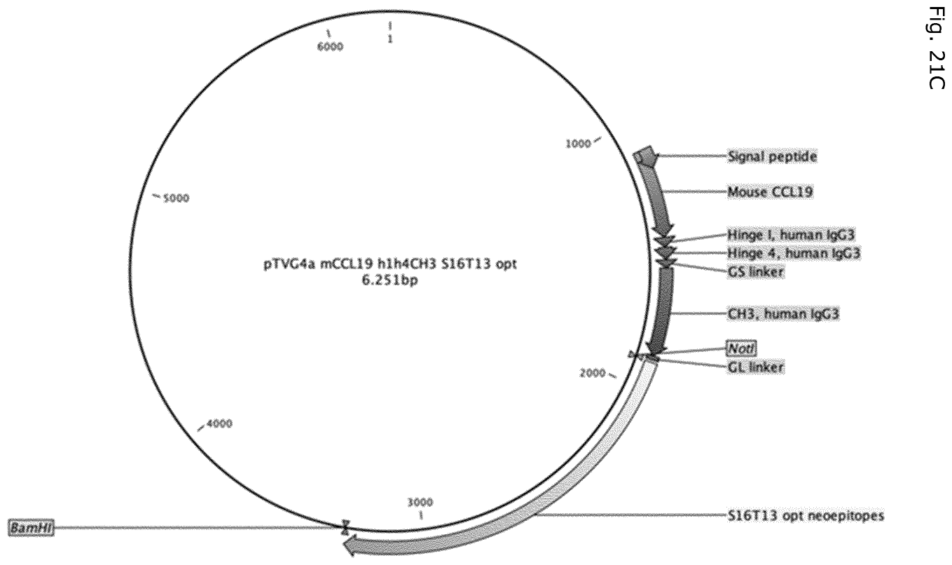

Splenic cells were re-stimulated with five of 27mer neo-peptides (C22, C23, C25, C30, C38) corresponding to the neo-epitope content of the S16A vector. Double-cytokine (INFγ and TNFα producing CD8+T and CD4+ cells were observed in groups immunized with a construct or combination of constructs harbouring neoepitopes. Immunization with a monomeric version of the fusion protein induces similar levels double positive CD8+ and CD4+T cells when compared to the dimeric version. Co-delivery of plasmids encoding for CCL19 and a secreted version of the neoepitopes turned out to be superior to the administration of a plasmid encoding secreted neoepitopes alone. Low or no signal was detected in negative control samples, comparable to or below background. See FIGS. 19A-19C for data.

Further, the DNA vaccines were well-tolerated by the mice; no signs of adverse effects were observed, and the body weight of the mice continuously increased throughout the study, as evident from the increase in body weight change, indicative of healthy and unaffected mice.

Assessment of body weight change for mice immunized with DNA
encoding combination/dissociation of the separate units for
the APC targeting technology for delivery of neoepitopes

| | Empty plasmid | | | mCCL19_S16A dimer | | | mCCL19_S16A monomer | | |
|---|---|---|---|---|---|---|---|---|---|
| Days | mean | SD | N | mean | SD | N | mean | SD | N |
| −14 | 0 | 0 | 13 | 0 | 0 | 13 | 0 | 0 | 13 |
| −13 | 0.2479374 | 1.885907 | 13 | 0.8746918 | 2.0789501 | 13 | 0.1278129 | 2.3391707 | 13 |
| −10 | 0.2764575 | 3.6292702 | 13 | −1.438595 | 2.359862 | 13 | −0.252501 | 2.9117128 | 13 |
| −7 | 4.0769331 | 2.9879401 | 13 | 1.151358 | 3.1574658 | 13 | 3.5386661 | 2.6480586 | 13 |
| −5 | 4.5127835 | 2.8127309 | 13 | 1.0586818 | 3.412296 | 13 | 4.1373635 | 3.2804711 | 13 |
| −3 | 4.766386 | 2.3286143 | 13 | 0.6268969 | 3.0403871 | 13 | 3.7652486 | 3.7242678 | 13 |
| 0 | 5.5395726 | 2.8854379 | 13 | 2.1639226 | 2.906687 | 13 | 5.4709868 | 3.1712732 | 13 |
| 1 | 6.8068808 | 3.0850282 | 13 | 2.8658581 | 3.4593078 | 13 | 6.1834534 | 3.3165246 | 13 |
| 4 | 7.0636339 | 3.6822052 | 13 | 3.0011873 | 4.279804 | 13 | 6.8730869 | 3.0296104 | 13 |
| 7 | 8.3959052 | 3.6970511 | 13 | 2.4068579 | 4.0702307 | 13 | 6.2602932 | 2.6446528 | 13 |
| 9 | 9.6960336 | 4.190404 | 13 | 4.2797275 | 4.0123717 | 13 | 7.0100454 | 2.8612889 | 13 |
| 11 | 11.120657 | 4.5962658 | 13 | 4.496152 | 4.1939636 | 13 | 7.668049 | 2.8675434 | 13 |
| 15 | 13.102226 | 5.2525782 | 13 | 7.9140611 | 5.2474548 | 13 | 9.9850326 | 3.0585043 | 13 |
| 16 | 14.407905 | 5.4190699 | 13 | 8.4805843 | 4.8019046 | 11 | 11.626924 | 3.7116911 | 12 |
| 18 | 13.254147 | 5.5683011 | 13 | 10.344788 | 4.3066984 | 11 | 10.378468 | 3.3180765 | 12 |
| 21 | 15.940184 | 3.4543543 | 11 | 9.2196447 | 3.951199 | 11 | 10.189654 | 3.0927191 | 12 |
| | | | | mCCL19 + | | | | | |
| | mCCL19 + S16A | | | SecSig only_S16A | | | SecSig only_S16A | | |
| Days | mean | SD | N | mean | SD | N | mean | SD | N |
| −14 | 0 | 0 | 13 | 0 | 0 | 13 | 0 | 0 | 13 |
| −13 | 0.3363469 | 2.849276 | 13 | −0.116557 | 2.6235486 | 13 | −0.309505 | 1.8179041 | 13 |
| −10 | 0.5399017 | 3.6302872 | 13 | 0.6659715 | 2.2319833 | 13 | −0.856511 | 2.7405685 | 13 |
| −7 | 2.046673 | 3.1052509 | 13 | 1.9990015 | 2.4132224 | 13 | 2.0901239 | 2.6036258 | 13 |
| −5 | 1.2998661 | 3.9683796 | 13 | 2.3140846 | 3.5211091 | 13 | 1.9946168 | 3.0938226 | 13 |
| −3 | 1.7950791 | 4.7893222 | 13 | 2.3299099 | 3.8583017 | 13 | 2.28857 | 3.1360442 | 13 |
| 0 | 4.2355916 | 4.8691098 | 13 | 3.5191472 | 4.8268418 | 13 | 4.7952941 | 4.1107589 | 13 |
| 1 | 5.5265159 | 5.4295635 | 13 | 4.4592385 | 4.6489957 | 13 | 5.3686055 | 4.6343427 | 13 |
| 4 | 6.9732772 | 5.7256578 | 13 | 5.0415309 | 4.9076359 | 13 | 5.6706553 | 4.77519 | 13 |
| 7 | 7.7884873 | 5.3121054 | 13 | 4.9024561 | 4.5869409 | 13 | 6.3273279 | 4.3344147 | 13 |
| 9 | 10.113595 | 6.0080605 | 13 | 7.5243626 | 4.3852818 | 13 | 8.2943677 | 5.1991386 | 13 |
| 11 | 11.198316 | 6.6055411 | 13 | 8.363441 | 4.4455726 | 13 | 8.6891917 | 4.9273341 | 13 |
| 15 | 13.045409 | 6.9021566 | 13 | 11.316722 | 3.9478518 | 13 | 9.7067576 | 5.8957347 | 13 |
| 16 | 13.006024 | 5.1353393 | 13 | 11.428477 | 5.0110762 | 12 | 11.375989 | 5.3506485 | 12 |
| 18 | 13.472109 | 5.0797797 | 13 | 11.816541 | 4.8421463 | 12 | 12.286157 | 5.4958607 | 12 |
| 21 | 14.609935 | 5.0919702 | 12 | 12.123724 | 6.9178058 | 10 | 12.880792 | 5.7666514 | 12 |
| | mCCL19 | | | S16A | | | Naïves | | |
| Days | mean | SD | N | mean | SD | N | mean | SD | N |
| −14 | 0 | 0 | 13 | 0 | 0 | 13 | 0 | 0 | 4 |
| −13 | 0.2011712 | 2.0668417 | 13 | 0.6493899 | 1.8358658 | 13 | 1.9587938 | 3.0031867 | 4 |
| −10 | 0.3715118 | 2.1207847 | 13 | −0.771033 | 2.46286 | 13 | −1.984108 | 0.9269933 | 4 |
| −7 | 3.2748973 | 2.60265 | 13 | 2.4137476 | 3.2404891 | 13 | 1.1103235 | 3.0020634 | 4 |
| −5 | 2.7416809 | 2.9657343 | 13 | 2.1622042 | 3.5767841 | 13 | 1.664205 | 6.8774572 | 4 |
| −3 | 3.069653 | 3.2998084 | 13 | 1.4646322 | 3.8788568 | 13 | −0.146879 | 6.1310353 | 4 |
| 0 | 3.7361795 | 4.2160861 | 13 | 2.4367049 | 3.8790624 | 13 | −0.873845 | 5.1622466 | 4 |
| 1 | 4.7030953 | 4.9425986 | 13 | 1.3353305 | 3.155075 | 13 | 0.5438803 | 4.6703385 | 4 |
| 4 | 7.0207128 | 5.0954049 | 13 | 3.2412657 | 3.5901768 | 13 | 0.8475327 | 5.8467054 | 4 |
| 7 | 7.5854634 | 4.6983045 | 13 | 4.7469959 | 4.7736347 | 13 | −1.028012 | 4.4306542 | 4 |
| 9 | 9.8063817 | 6.215638 | 13 | 7.3217606 | 4.9660625 | 13 | 3.2754155 | 5.942024 | 4 |
| 11 | 10.064959 | 7.3266931 | 13 | 6.4435091 | 4.9761201 | 13 | 1.9369119 | 5.1442084 | 4 |
| 15 | 12.105985 | 6.1545446 | 13 | 8.3733404 | 4.2085153 | 13 | 4.9621835 | 6.5217102 | 4 |
| 16 | 13.613458 | 4.8249237 | 13 | 12.170933 | 4.5587239 | 13 | 10.136396 | 6.4251012 | 4 |
| 18 | 13.58406 | 6.0444586 | 12 | 11.298329 | 4.7964645 | 13 | 8.4741938 | 7.0103076 | 4 |
| 21 | 14.558129 | 6.2365685 | 11 | 12.104161 | 4.5924463 | 11 | 6.3500841 | 5.749858 | 4 |

Conclusions

The Kolliphor delivered pUMVC4 plasmid vectors containing mCCL19, different multimerization units and the S16A neoepitopes resulted in CT26 anti-tumour effects from 50-100%. The groups receiving a DNA design with a multimerization unit that was of Ig origin or collagen performed the best. The neoepitope specific CD8+ T cell response at day 7 corresponded to the antitumor effects and the level of functional T cells at endpoint (as measured by IFNγ secretion) was comparable between groups receiving a DNA design containing neoepitopes. Furthermore the DNA vaccines were well-tolerated by the mice.

### EXAMPLE 5

Use of alternative delivery plasmid

The efficacy of the APC targeting constructs was tested in an alternative vector backbone (the pTVG4 plasmid). It was further tested whether the inclusion of additional neoepitopes (to arrive at 13 in total) has any impact on the efficacy. Finally, it was investigated whether minor changes—such as addition of a restriction site upstream of the neoepitope encoding region—affect the transcription and in vivo efficacies.

Study plan

The vector used in this experiment (pTVG4, see FIG. 21A) is, as is the case for pUMVC4, build on the basis of pUMVC3. A version of pTVG4 was engineered by addition of murine CCL19 encoding material (SEQ ID NO: 39) and of BamHI and NotI restriction sites surrounding the site for introducing the neoepitope coding region (cf. FIGS. 21B and 21C where the vector is shown without and including the 516T13 epitope encoding insert). A corresponding vector was also produced where the murine CCL19 encoding sequence was exchanged for the human counterpart (encoded protein: Uniprot entry no. Q99731). See FIG. 21D.

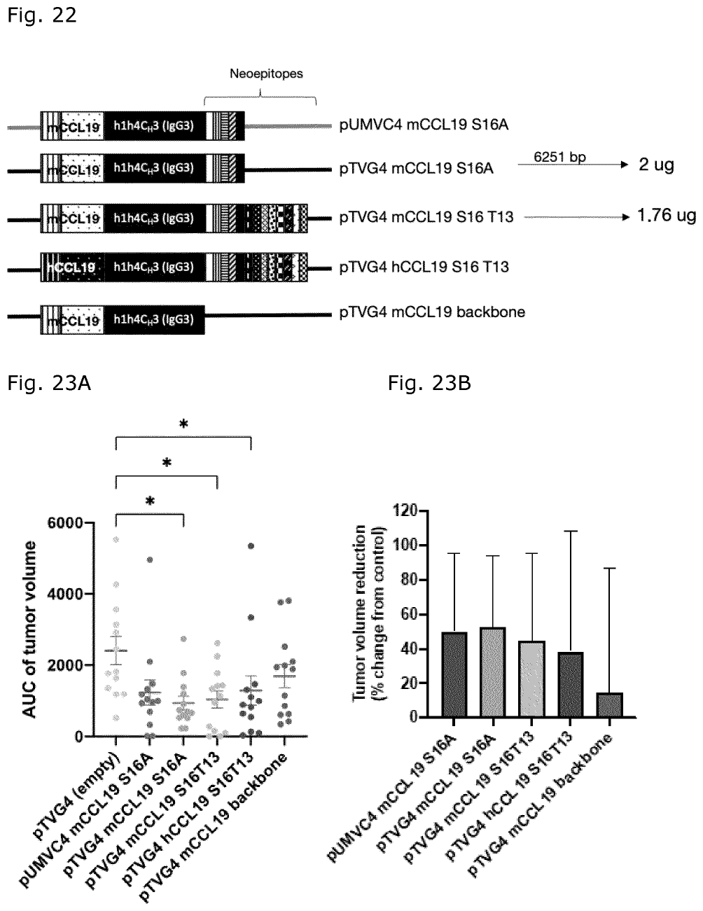

The DNA cassettes tested are schematically depicted in FIG. 22. One pUMVC4-based construct is compared to a number of pTVG4 constructs. The 13 epitopes are, from the N-terminus, C22, C23, C38, C25, C30, C37, EV85, C40, C41, C29, EV22, EV105, and AA427 (cf. the sequences set forth below).

CT26 mice were immunized according to the following sheme

Group 1: Untreated/Vehicle, n=13

Group 2: pTVG4 (empty), n=13

Group 2: pUMVC4 mCCL19 516A, n=13

Group 3: pTVG4 mCCL19 516A, n=13

Group 4: pTVG4 mCCL19 S16 T13, n=13

Group 5: pTVG4 hCCL19 S16 T13, n=13

Group 6: pTVG4 mCCL19 backbone, n=13

Group 7: Naïve mice, n=5

Immunizations were administered i.m. on days -13, −6, 1, 8, and 15 relative to challenge with CT26 tumour cells (day 0). Blood was withdrawn from the tail vein on days 2 and 5. At the end of the experiment (day 21) mice where euthanized and spleens recovered and tumours excised.

Read-outs were 1) change in body weight (BW) from the time of first immunization, 2) tumour volume (TV), and 3) C22 neo-epitope specific T-cells in circulation.

Results

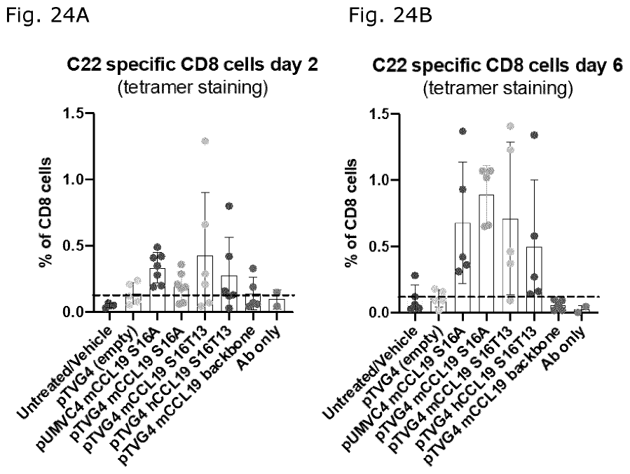

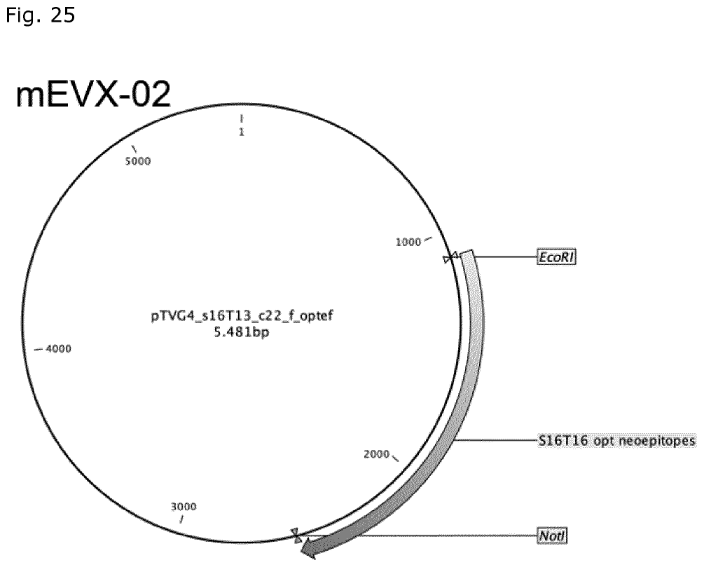

Results are shown in FIG. 23 and FIG. 24.

With reference to FIG. 23, The pTVG4 backbone performs just as well (or better) as the pUMVC4 backbone in affecting tumour growth and the addition of the restriction sites does not negatively affect the antitumor effect. Antitumour activity is not negatively affected by addition of further neo-epitopes and the human CCL19 as targeting unit is as efficient as murine CCL19 in the CT26 mouse tumour model.

With reference to FIG. 24, CD8+T cells specific to CT26 neopeptide C22 (H-2Kd minimal binder KFKASRASI; SEQ ID NO: 61) were observed in tail vein blood at study day 2 and 6 in mice immunized with pUMVC4 vector containing the S16A neoepitopes and in pTVG4 constructs containing S16A or 516T13 neoepitopes.

Furthermore the DNA vaccines were well-tolerated by the mice as evidence by the BW data.

Conclusions

The pTVG4 backbone was shown to performs as well as the pUMVC4, and restriction site introduction did not negatively affect the antitumor effect or T cell response. Furthermore, addition of 13 neoepitopes instead of 5 does not affect the antitumor effect. Finally, it also can be concluded the human CCL19 for use as targeting unit is as efficient as murine CCL19 in the CT26 mouse tumour model.

### EXAMPLE 6

Comparison of vaccine constructs with and without APC targeting unit, respectively

The objective was to perform a direct comparison between two plasmid constructs (mEVX-03 and mEVX-02; see FIGS. and 21C, respectively), which are both based on the pTVG4 vector backbone, in a mouse model to investigate the antigen targeting unit's effect on anti-tumour effect and T cell response induction.

Study Plan

A dose titration for both compounds constructs was undertaken. Both DNA preparations for mEVX-03 and mEVX-02 was high-grade plasmid and was formulated with the same polymer P188 in PBS. Both vectors encode the 13 neoepitopes set forth in Example 5.

Immunizations were administered i.m. on days -13, −6, 1, 8, and 15 relative to challenge with CT26 tumour cells (day 0). Blood was withdrawn from the tail vein on days 6 and 16. At the end of the experiment (day 21) mice were euthanized, spleens recovered and tumours excised. All groups of mice consisted of 13 animals.

Doses of test vaccines were as follows:

| Group | Treatment | Dose (ug) |
|---|---|---|
| 1 | mEVX-02 | 5 |
| 2 | mEVX-02 | 2 |
| 3 | mEVX-02 | 1 |
| 4 | mEVX-02 | 0.5 |
| 5 | mEVX-02 | 0.25 |
| 6 | mEVX-03 | 5 |
| 7 | mEVX-03 | 2 |
| 8 | mEVX-03 | 1 |
| 9 | mEVX-03 | 0.5 |
| 10 | mEVX-03 | 0.25 |
| 11 | Empty vector | 5 |
| | (pTVG4) | |

Read-outs were 1) Body weight (BW) change from first immunization, 2) Tumor volume (TV), and 3) C22 neo-epitope specific CD8+ T cells in circulation.

Results

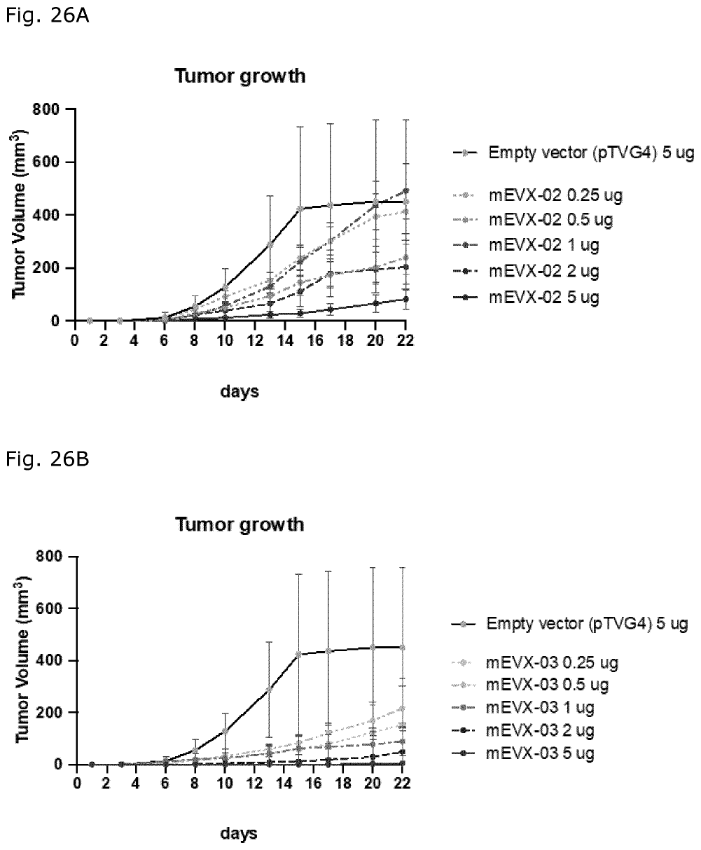

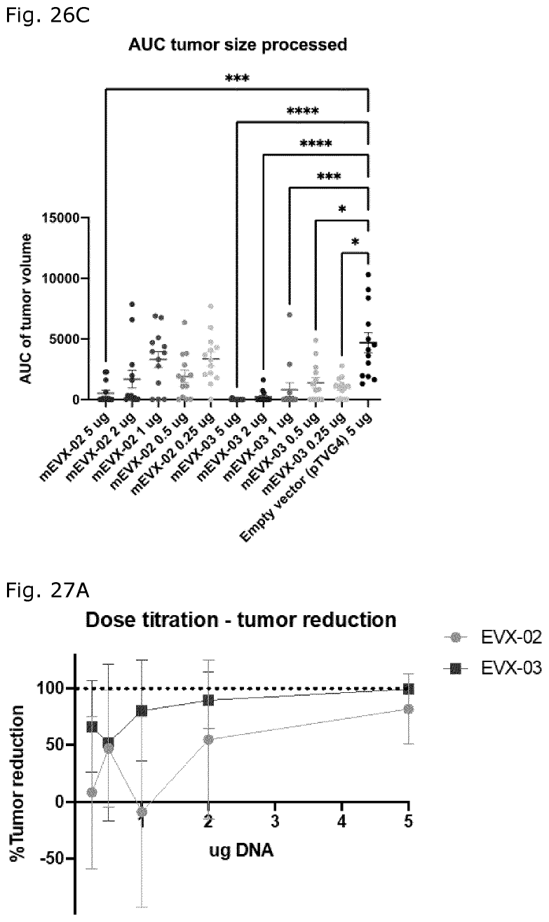

The effect on tumour growth exerted by each construct at various dosages is set forth in FIG. 26A (EVX-02) and 26B (EVX-03). A clear improvement is observed when immunizing with EVX-03 (see FIG. 26C): The highest dose of mEVX-02 elicited a significant anti-tumour response, whereas all tested doses of mEVX-03 elicited a significant anti-tumour response.

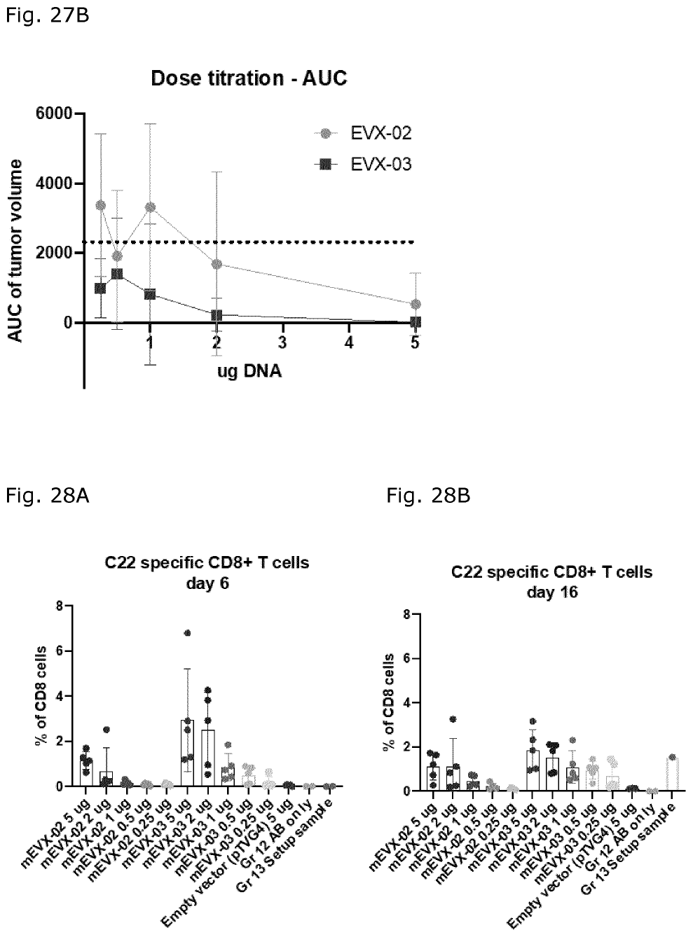

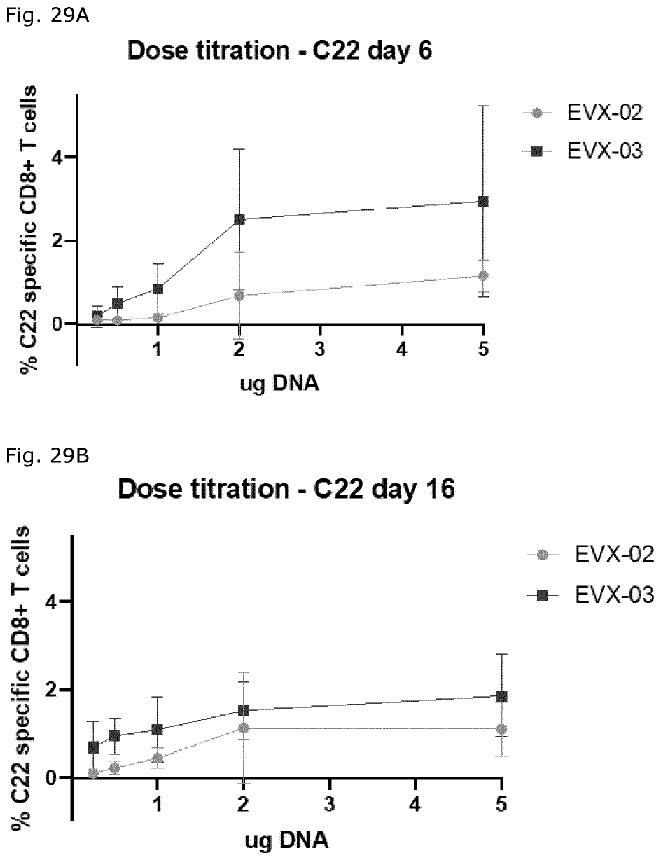

When evaluated vs. dosing of the plasmid constructs, the mEVX-03 also exhibited an clear (albeit insignificant) improvement over mEVX-02, cf. FIGS. 27A and 27B: at all doses tested, EVX-03 was more effective than EVX-02.

With respect to induction of C22 specific CD8+T cells, these were observed in tail vein blood at study day 6 and 16 in mice prophylactically immunized with mEVX-02 and mEVX-03 at different doses (see FIGS. 28A and 28B). mEVX-03, however was more potent in eliciting a neoepitope specific T cell response than mEVX-02 at the same dose, see FIGS. 29A and 29B. No tetramer signal was detected in control samples (naïve mice and empty vector immunized mice).

The animals tolerated the DNA immunizations well.

Conclusions

A head-to-head comparison between immunization with two constructs distinguished only in the presence of an antigen presenting cell targeting unit according to the present invention confirms that the inclusion of the APC targeting unit allows for use of lower dosages of plasmid.

BIOLOGICAL SEQUENCES

SEQ ID NO: 1:
GSGGGA
SEQ ID NO: 2:
GSGGGAGSGGGA
SEQ ID NO: 3:
GSGGGAGSGGGAGSGGGA
SEQ ID NO: 4:
GSGGGAGSGGGAGSGGGAGSGGGA
SEQ ID NO: 5:
GENLYFQSGG
SEQ ID NO: 6:
KPEPKPAPAPKP
SEQ ID NO: 7:
AEAAAKEAAAKA
SEQ ID NO: 8:
SACYCELS
SEQ ID NO: 9:
SGGGSSGGGS
SEQ ID NO: 10:
GGGGSGGGGS
SEQ ID NO: 11:
SSGGGSSGGG
SEQ ID NO: 12:
GGSGGGGSGG
SEQ ID NO: 13:
GSGSGSGSGS
SEQ ID NO: 14:
ggGGTCAACGTTGAgggggg
SEQ ID NO: 15:
ggGGGACGATCGTCgggggg
SEQ ID NO: 16:
gggGACGACGTCGTGgggggg
SEQ ID NO: 17:
tccatgacgttcctgatgct
SEQ ID NO: 18:
tccatgacgttcctgacgtt
SEQ ID NO: 19:
tcgtcgttttgtcgttttgtcgtt
SEQ ID NO:20:
tcgtcgttgtcgttttgtcgtt
SEQ ID NO: 21:
tcgacgttcgtcgttcgtcgttc
SEQ ID NO: 22:
tcgcgacgttcgcccgacgttcggta
SEQ ID NO: 23:
tcgtcgttttcggcgcgcgccg
SEQ ID NO: 24:
tcgtcgtcgttcgaacgacgttgat
SEQ ID NO: 25:
tcgcgaacgttcgccgcgttcgaacgcgg
SEQ ID NO: 26:
agatctaacg acaaaacgac aaaacgacaa ggcgccagat ctggcgtttc gttttgtcgt
tttgtcgtta gatct
SEQ ID NO: 27:
GGTGCATCGA TGCAGGGGGG
SEQ ID NO: 28:
GGTGCATCGA TGCAGGGGGG TATATATATA TTGAGGACAG GTTAAGCTCC CCCCAGCTTA
ACCTGTCCTT CAATATATA TATA
SEQ ID NO: 29, vectgor backbone; pUMVC4a from Aldevron:
tggccattgc atacgttgta tccatatcat aatatgtaca tttatattgg ctcatgtcca
acattaccgc catgttgaca ttgattattg actagttatt aatagtaatc aattacgggg
tcattagttc atagcccata tatggagttc cgcgttacat aacttacggt aaatggcccg
cctggctgac cgcccaacga cccccgccca ttgacgtcaa taatgacgta tgttcccata
gtaacgccaa tagggacttt ccattgacgt caatgggtgg agtatttacg gtaaactgcc
cacttggcag tacatcaagt gtatcatatg ccaagtacgc cccctattga cgtcaatgac
ggtaaatggc ccgcctggca ttatgcccag tacatgacct tatgggactt tcctacttgg
cagtacatct acgtattagt catcgctatt accatggtga tgcggttttg gcagtacatc
aatgggcgtg gatagcggtt tgactcacgg ggatttccaa gtctccaccc cattgacgtc
aatgggagtt tgttttggca ccaaaatcaa cgggactttc caaaatgtcg taacaactcc
gccccattga cgcaaatggg cggtaggcgt gtacggtggg aggtctatat aagcagagct
cgtttagtga accgtcagat cgcctggaga cgccatccac gctgttttga cctccataga
agacaccggg accgatccag cctccgcggc cgggaacggt gcattggaac gcggattccc
cgtgccaaga gtgacgtaag taccgcctat agagtctata ggcccacccc cttggcttct
tatgcatgct atactgtttt tggcttgggg tctatacacc cccgcttcct catgttatag
gtgatggtat agcttagcct ataggtgtgg gttattgacc attattgacc actccaacgg
tggagggcag tgtagtctga gcagtactcg ttgctgccgc gcgcgccacc agacataata
gctgacagac taacagactg ttcctttcca tgggtctttt ctgcagtcac cgtcgtcgac
ggtatcgata agcttgatat cgaattcacg tgggcccggt accgtatact ctagagcggc
cgcggatcca gatctttttc cctcgccaaa aattatgggg acatcatgaa gccccttgag
catctgactt ctggctaata aaggaaattt atttcattgc aatagtgtgt tggaattttt
tgtgtctctc actcggaagg acatatggga gggcaaatca tttaaaacat cagaatcagt
atttggttta gagtttggca acatatgcca ttcttccgct tcctcgctca ctgactcgct
gcgctcggtc gttcggctgc ggcgagcggt atcagctcac tcaaaggcgg taatacggtt
atccacagaa tcaggggata acgcaggaaa gaacatgtga gcaaaaggcc agcaaaaggc
caggaaccgt aaaaaggccg cgttgctggc gtttttccat aggctccgcc cccctgacga
gcatcacaaa aatcgacgct caagtcagag gtggcgaaac ccgacaggac tataaagata
ccaggcgttt ccccctggaa gctccctcgt gcgctctcct gttccgaccc tgccgcttac
cggatacctg tccgcctttc tcccttcggg aagcgtggcg ctttctcaat gctcacgctg
taggtatctc agttcggtgt aggtcgttcg ctccaagctg ggctgtgtgc acgaaccccc
cgttcagccc gaccgctgcg ccttatccgg taactatcgt cttgagtcca acccggtaag
acacgactta tcgccactgg cagcagccac tggtaacagg attagcagag cgaggtatgt
aggcggtgct acagagttct tgaagtggtg gcctaactac ggctacacta gaaggacagt
atttggtatc tgcgctctgc tgaagccagt taccttcgga aaaagagttg gtagctcttg
atccggcaaa caaaccaccg ctggtagcgg tggttttttt gtttgcaagc agcagattac
gcgcagaaaa aaaggatctc aagaagatcc tttgatcttt tctacggggt ctgacgctca
gtggaacgaa aactcacgtt aagggatttt ggtcatgaga ttatcaaaaa ggatcttcac
ctagatcctt ttaaattaaa aatgaagttt taaatcaatc taaagtatat atgagtaaac
ttggtctgac agttaccaat gcttaatcag tgaggcacct atctcagcga tctgtctatt
tcgttcatcc atagttgcct gactccgggg ggggggggcg ctgaggtctg cctcgtgaag
aaggtgttgc tgactcatac cagggcaacg ttgttgccat tgctacaggc atcgtggtgt
cacgctcgtc gtttggtatg gcttcattca gctccggttc ccaacgatca aggcgagtta
catgatcccc catgttgtgc aaaaaagcgg ttagctcctt cggtcctccg atcgttgtca
gaagtaagtt ggccgcagtg ttatcactca tggttatggc agcactgcat aattctctta
ctgtcatgcc atccgtaaga tgcttttctg tgactggtga gtactcaacc aagtcattct
gagaatagtg tatgcggcga ccgagttgct cttgcccggc gtcaatacgg gataataccg
cgccacatag cagaacttta aaagtgctca tcattggaaa acgttcttcg gggcgaaaac
tctcaaggat cttaccgctg ttgagatcca gttcgatgta acccactcgt gcacctgaat
cgccccatca tccagccaga aagtgaggga gccacggttg atgagagctt tgttgtaggt
ggaccagttg gtgattttga acttttgctt tgccacggaa cggtctgcgt tgtcgggaag
atgcgtgatc tgatccttca actcagcaaa agttcgattt attcaacaaa gccgccgtcc
cgtcaagtca gcgtaatgct ctgccagtgt tacaaccaat taaccaattc tgattagaaa
aactcatcga gcatcaaatg aaactgcaat ttattcatat caggattatc aataccatat
ttttgaaaaa gccgtttctg taatgaagga gaaaactcac cgaggcagtt ccataggatg
gcaagatcct ggtatcggtc tgcgattccg actcgtccaa catcaataca acctattaat
ttcccctcgt caaaaataag gttatcaagt gagaaatcac catgagtgac gactgaatcc
ggtgagaatg gcaaaagctt atgcatttct ttccagactt gttcaacagg ccagccatta
cgctcgtcat caaaatcact cgcatcaacc aaaccgttat tcattcgtga ttgcgcctga
gcgagacgaa atacgcgatc gctgttaaaa ggacaattac aaacaggaat cgaatgcaac
cggcgcagga acactgccag cgcatcaaca atattttcac ctgaatcagg atattcttct
aatacctgga atgctgtttt cccggggatc gcagtggtga gtaaccatgc atcatcagga
gtacggataa aatgcttgat ggtcggaaga ggcataaatt ccgtcagcca gtttagtctg
accatctcat ctgtaacatc attggcaacg ctacctttgc catgtttcag aaacaactct
ggcgcatcgg gcttcccata caatcgatag attgtcgcac ctgattgccc gacattatcg
cgagcccatt tatacccata taaatcagca tccatgttgg aatttaatcg cggcctcgag
caagacgttt cccgttgaat atggctcata acaccccttg tattactgtt tatgtaagca
gacagtttta ttgttcatga tgatatattt ttatcttgtg caatgtaaca tcagagattt
tgagacacaa cgtggctttc cccccccccc cattattgaa gcatttatca gggttattgt
ctcatgagcg gatacatatt tgaatgtatt tagaaaaata aacaaatagg ggttccgcgc
acatttcccc gaaaagtgcc acctgacgtc taagaaacca ttattatcat gacattaacc
tataaaaata ggcgtatcac gaggcccttt cgtcctcgcg cgtttcggtg atgacggtga
aaacctctga cacatgcagc tcccggagac ggtcacagct tgtctgtaag cggatgccgg
gagcagacaa gcccgtcagg gcgcgtcagc gggtgttggc gggtgtcggg gctggcttaa
ctatgcggca tcagagcaga ttgtactgag agtgcaccat atgcggtgtg aaataccgca
cagatgcgta aggagaaaat accgcatcag attggctat
SEQ ID NO: 30, enhancer, start Kozak sequence without ATG:
CGCCGCCACC
SEQ ID NO: 31, dimer Hinge 1, human IgG3:
GAGCTCAAAA CCCCACTTGG TGACACAACT CACACA
SEQ ID NO: 32, dimer Hinge 4, human IgG3:
GAGCCCAAAT CTTGTGACAC ACCTCCCCCG TGCCCAAGGT GCCCA
SEQ ID NO:33, dimer Gly-ser linker:
GGCGGTGGAA GCAGCGGAGG TGGAAGTGGA
SEQ ID NO: 34, dimer CH3, human IgG3:
GGACAGCCCC GAGAACCACA GGTGTACACC CTGCCCCCAT CCCGGGAGGA GATGACCAAG
AACCAGGTCA GCCTGACCTG CCTGGTCAAA GGCTTCTACC CCAGCGACAT CGCCGTGGAG
TGGGAGAGCA GCGGGCAGCC GGAGAACAAC TACAACACCA CGCCTCCCAT GCTGGACTCC
GACGGCTCCT TCTTCCTCTA CAGCAAGCTC ACCGTGGACA AGAGCAGGTG GCAGCAGGGG
AACATCTTCT CATGCTCCGT GATGCATGAG GCTCTGCACA ACCGCTTCAC GCAGAAGAGC
CTCTCCCTGT CTCCGGGTAA A
SEQ ID NO: 35, dimer Gly-Leu linker:
GGCCTCGGTG GCCTG
SEQ ID NO: 36, APC targeting mCCL3:
ATGAAGGTCT CCACCACTGC CCTTGCTGTT CTTCTCTGTA CCATGACACT CTGCAACCAA
GTCTTCTCAG CGCCATATGG AGCTGACACC CCGACTGCCT GCTGCTTCTC CTACAGCCGG
AAGATTCCAC GCCAATTCAT CGTTGACTAT TTTGAAACCA GCAGCCTTTG CTCCCAGCCA
GGTGTCATTT TCCTGACTAA GAGAAACCGG CAGATCTGCG CTGACTCCAA AGAGACCTGG
GTCCAAGAAT ACATCACTGA CCTGGAACTG AATGCC
SEQ ID NO: 37. APC targeting mCCL4:
ATGAAGGTCT GCGTGTCTGC CCTCTCTCTC CTCTTGCTCG TGGCTGCCTT CTGTGCTCCA
GGGTTCTCAG CACCAATGGG CTCTGACCCT CCCACTTCCT GCTGTTTCTC TTACACCTCC
CGGCAGCTTC ACAGAAGCTT TGTGATGGAT TACTATGAGA CCAGCAGTCT TTGCTCCAAG
CCAGCTGTGG TATTCCTGAC CAAAAGAGGC AGACAGATCT GTGCTAACCC CAGTGAGCCC
TGGGTCACTG AGTACATGAG TGACTTGGAG
SEQ ID NO: 38, APC targeting mCCL5:
ATGAAGATCT CTGCAGCTGC CCTCACCATC ATCCTCACTG CAGCCGCCCT CTGCACCCCC
GCACCTGCCT CACCATATGG CTCGGACACC ACTCCCTGCT GCTTTGCCTA CCTCTCCCTC
GCGCTGCCTC GTGCCCACGT CAAGGAGTAT TTCTACACCA GCAGCAAGTG CTCCAATCTT
GCAGTCGTGT TTGTCACTCG AAGGAACCGC CAAGTGTGTG CCAACCCAGA GAAGAAGTGG
GTTCAAGAAT ACATCAACTA TTTGGAGATG AGC
SEQ ID NO: 39, APC targeting mCCL19:
ATGGCCCCCC GTGTGACCCC ACTCCTGGCC TTCAGCCTGC TGGTTCTCTG GACCTTCCCA
GCCCCAACTC TGGGGGGTGC TAATGATGCG GAAGACTGCT GCCTGTCTGT GACCCAGCGC
CCCATCCCTG GGAACATCGT GAAAGCCTTC CGCTACCTTC TTAATGAAGA TGGCTGCAGG
GTGCCTGCTG TTGTGTTCAC CACACTAAGG GGCTATCAGC TCTGTGCACC TCCtGACCAG
CCCTGGGTGG ATCGCATCAT CCGAAGACTG AAGAAGTCTT CTGCCAAGAA CAAAGGCAAC
AGCACCAGAA GGAGCCCTGT GTCT
SEQ ID NO: 40, APC targeting mXcl1:
ATGAGACTTC TCCTCCTGAC TTTCCTGGGA GTCTGCTGCC TCACCCCATG GGTTGTGGAA
GGTGTGGGGA CTGAAGTCCT AGAAGAGAGT AGCTGTGTGA ACTTACAAAC CCAGCGGCTG
CCAGTTCAAA AAATCAAGAC CTATATCATC TGGGAGGGGG CCATGAGAGC TGTAATTTTT
GTCACCAAAC GAGGACTAAA AATTTGTGCT GATCCAGAAG CCAAATGGGT GAAAGCAGCG
ATCAAGACTG TGGATGGCAG GGCCAGTACC AGAAAGAACA TGGCTGAAAC TGTTCCCACA
GGAGCCCAGA GGTCCACCAG CACAGCGATA ACCCTGACTG GG
SEQ ID NO: 41, APC targeting mCCL20:
ATGGCCTGCG GTGGCAAGCG TCTGCTCTTC CTTGCTTTGG CATGGGTACT GCTGGCTCAC
CTCTGCAGCC AGGCAGAAGC AGCAAGCAAC TACGACTGTT GCCTCTCGTA CATACAGACG
CCTCTTCCTT CCAGAGCTAT TGTGGGTTTC ACAAGACAGA TGGCCGATGA AGCTTGTGAC
ATTAATGCTA TCATCTTTCA CACGAAGAAA AGAAAATCTG TGTGCGCTGA TCCAAAGCAG
AACTGGGTGA AAAGGGCTGT GAACCTCCTC AGCCTAAGAG TCAAGAAGAT G
SEQ ID NO: 42, APC targeting mCCL21:
ATGGCTCAGA TGATGACTCT GAGCCTCCTT AGCCTGGTCC TGGCTCTCTG CATCCCCTGG
ACCCAAGGCA GTGATGGAGG GGGTCAGGAC TGCTGCCTTA AGTACAGCCA GAAGAAAATT
CCCTACAGTA TTGTCCGAGG CTATAGGAAG CAAGAACCAA GTTTAGGCTG TCCCATCCCG
GCAATCCTGT TCTCACCCCG GAAGCACTCT AAGCCTGAGC TATGTGCAAA CCCTGAGGAA
GGCTGGGTGC AGAACCTGAT GCGCCGCCTG GACCAGCCTC CAGCCCCAGG GAAACAAAGC
CCCGGCTGCA GGAAGAACCG GGGAACCTCT AAGTCTGGAA AGAAAGGAAA GGGCTCCAAG
GGCTGCAAGA GAACTGAACA GACACAGCCC TCAAGAGGA
SEQ ID NO: 43, APC targeting GM-CSF:
ATGTGGCTGC AGAATTTACT TTTCCTGGGC ATTGTGGTCT ACAGCCTCTC AGCACCCACC
CGCTCACCCA TCACTGTCAC CCGGCCTTGG AAGCATGTAG AGGCCATCAA AGAAGCCCTG
AACCTCCTGG ATGACATGCC TGTCACGTTG AATGAAGAGG TAGAAGTCGT CTCTAACGAG
TTCTCCTTCA AGAAGCTAAC ATGTGTGCAG ACCCGCCTGA AGATATTCGA GCAGGGTCTA
CGGGGCAATT TCACCAAACT CAAGGGCGCC TTGAACATGA CAGCCAGCTA CTACCAGACA
TACTGCCCCC CAACTCCGGA AACGGACTGT GAAACACAAG TTACCACCTA TGCGGATTTC
ATAGACAGCC TTAAAACCTT TCTGACTGAT ATCCCCTTTG AATGCAAAAA ACCAGGCCAA AAA
SEQ ID NO: 44, secretion signal on APC t Secretion signal, mouse serum
albumin:
ATGAAATGGG TGACCTTTCT GCTGCTGCTG TTTGTGAGCG GCAGCGCGTT TAGCG
SEQ ID NO: 45, APC targeting anti-murine DEC-205 Fv:
CGGCCCAGCC GGCCATGGCG GACTACAAAC AGGCTGTTGT GACTCAGGAA TCAGCACTCA
CCACATCACC TGGTGAAACA GTCACACTCA CTTGTCGCTC AAGTACTGGG GCTGTTACAA
TTAGTAACTA TGCCAACTGG GTCCAAGAAA AACCAGATCA TTTATTCACT GGTCTAATAG
GTGGTACCAA CAACCGAGCT CCAGGTGTTC CTGCCAGATT CTCAGGCTCC CTGATTGGAG
ACAAGGCTGC CCTCACCATC ACAGGGGCAC AGACTGAGGA TGAGGCAATC TATTTCTGTG
CTCTATGGTA CAACAACCAG TTTATTTTCG GCAGTGGAAC CAAGGTCACT GTCCTAGGTG
GTGGTGGTGG TTCTGGTGGT GGTGGATCCG GCGGCGGCGG CTCTGGCGGC GGCGGCTCTG
AGGTCCAGCT GCAACAGTCT GGACCTGTGC TGGTGAAGCC TGGGGCTTCA GTGAAGATGT
CCTGTAAGGC TTCTGGAAAC ACATTCACTG ACTCCTTTAT GCACTGGATG AAACAGAGCC
ATGGAAAGAG TCTTGAGTGG ATTGGAATTA TTAATCCTTA TAACGGCGGT ACTAGCTACA
ACCAGAAATT CAAGGGCAAG GCCACATTGA CTGTTGACAA GTCCTCCAGC ACAGCCTACA
TGGAGCTCAA CAGCCTGACA TCTGAGGACT CTGCAGTCTA TTACTGTGCA AGAAACGGGG
TGCGGTACTA CTTTGACTAC TGGGGCCAAG GCACCACTCT CACAGTCTCC TCGGCCTCGG
GGGCC
SEQ ID NO: 46, APC targeting anti-murine CLEC9 Fv:
GCGGCCCAGC CGGCCATGGC GGACTACAAA CAGGCTGTTG TGACTCAGGA ATCAGCACTC
ACCACATCAC CTGGTGAAAC AGTCACACTC ACTTGTCGCT CAAGTAAAAG CAGCCAGAGC
GTGCTGTATG ATGAAAACAA AAAAAACTAT CTGGCCAACT GGGTCCAAGA AAAACCAGAT
CATTTATTCA CTGGTCTAAT AGGTTGGGCG AGCACCGGCG AAAGCAACCG AGCTCCAGGT
GTTCCTGCCA GATTCTCAGG CTCCCTGATT GGAGACAAGG CTGCCCTCAC CATCACAGGG
GCACAGACTG AGGATGAGGC AATCTATTTC TGTTATTATG ATTTTCCGCC GTTCGGCAGT
GGAACCAAGG TCACTGTCCT AGGTGGTGGT GGTGGTTCTG GTGGTGGTGG ATCCGGCGGC
GGCGGCTCTG GCGGCGGCGG CTCTGAGGTC CAGCTGCAAC AGTCTGGACC TGTGCTGGTG
AAGCCTGGGG CTTCAGTGAA GATGTCCTGT AAGGCTTCTA ACGCGGCGAT TTATATGCAC
TGGATGAAAC AGAGCCATGG AAAGAGTCTT GAGTGGATTG GAATTCGCAT TCGCACCCGC
CCGAGCAAAT ATGCGACCGA TTATGCGGAT AGCGTGCGCG GCAGCTACAA CCAGAAATTC
AAGGGCAAGG CCACATTGAC TGTTGACAAG TCCTCCAGCA CAGCCTACAT GGAGCTCAAC
AGCCTGACAT CTGAGGACTC TGCAGTCTAT TACTGTCGCG CGACCGAAGA TGTGCCGTTT
TATTACTGGG GCCAAGGCAC CACTCTCACA GTCTCCTCGG CCTCGGGGGC C
SEQ ID NO: 47, APC targeting CLEC9 ligand:
TGGCCCAGGT TCCACAGCAG CGTGTTCCAC ACCCAC
SEQ ID NO: 48, APC targeting mFLT3L:
ATGACACCTG ACTGTTACTT CAGCCACAGT CCCATCTCCT CCAACTTCAA AGTGAAGTTT
AGAGAGTTGA CTGACCACCT GCTTAAAGAT TACCCAGTCA CTGTGGCCGT CAATCTTCAG
GACGAGAAGC ACTGCAAGGC CTTGTGGAGC CTCTTCCTAG CCCAGCGCTG GATAGAGCAA
CTGAAGACTG TGGCAGGGTC TAAGATGCAA ACGCTTCTGG AGGACGTCAA CACCGAGATA
CATTTTGTCA CCTCATGTAC CTTCCAGCCC CTACCAGAAT GTCTGCGATT CGTCCAGACC
AACATCTCCC ACCTCCTGAA GGACACCTGC ACACAGCTGC TTGCTCTGAA GCCCTGTATC
GGGAAGGCCT GCCAGAATTT CTCTCGGTGC CTGGAGGTGC AGTGCCAGCC GGACTCCTCC
ACCCTGCTGC CCCCAAGGAG TCCCATAGCC CTAGAAGCCA CGGAGCTCCC AGAGCCTCGG
CCCAGGCAG
SEQ ID NO: 49, dimer, dHLX protein:
GGAGAACTGG AGGAATTACT TAAACATCTC AAGGAGTTGC TCAAAGGCCC TAGGAAGGGA
GAACTGGAGG AACTCCTCAA ACATCTCAAG GAGTTACTAA AGGGA
SEQ ID NO: 50, dimer, GS-linker:
GGCAGCGGCA GCGGCAGCGG CAGCGGCAGC
SEQ ID NO: 51, dimer, hMHD2 (human IgM):
GCCGAACTCC CGCCCAAGGT GTCCGTGTTC GTCCCTCCCC GCGATGGGTT CTTCGGCAAT
CCACGAAAAT CCAAACTGAT TTGTCAGGCC ACCGGCTTCT CCCCCCGACA GATCCAGGTG
AGTTGGCTAC GAGAGGGTAA ACAGGTGGGG AGCGGAGTGA CCACTGACCA GGTGCAGGCC
GAGGCCAAGG AAAGCGGACC CACAACATAC AAAGTGACAA GCACTCTGAC GATTAAGGAG
TCAGACTGGC TCGGCCAATC CATGTTTACA TGCCGGGTTG ATCACAGAGG GTTGACCTTC
CAACAGAACG CATCCAGTAT GTGCGTTCCA GAT
SEQ ID NO: 52, Trimer Collagen trimerization domain:
GCTGGGCAGG TGAGGATCTG GGCCACATAC CAGACCATGC TGGACAAGAT CCGGGAGGTG
CCGGAGGGCT GGCTCATCTT TGTGGCCGAG AGGGAAGAGC TCTATGTACG CGTTAGAAAT
GGCTTCCGGA AGGTGCTGCT GGAGGCCCGG ACAGCCCTCC CGAGAGGCAC GGGCAATGAG
SEQ ID NO: 53, Tetramer p53 synthetic protein:
TTCCGGGAGC ACGGAGAGTA TTTCACTCTC CAGATCCGGG GCCCCGAAAG GTTCGAAATG
AAGCCTCTGG TTAAGGAGGC CTTGGAGCTG AAAGACGCAC AGGCCGGAAA
SEQ ID NO: 54:
QIETQQRKFK ASRASILSEM
SEQ ID NO: 55:
VILPQAPSGP SYATYLQPAQ AQMLTPP
SEQ ID NO: 56:
DTLSAMSNPR AMQVLLQIQQ GLQTLAT
SEQ ID NO: 57:
DGQLELLAQG ALDNALSSMG ALHALPR
SEQ ID NO: 58:
RLHWKLLAS ALSTNAAALT QELLVLD
SEQ ID NO: 59, dimer Hinge 1, human IgG3 Protein sequence:
ELKTPLGDTTHT
SEQ ID NO: 60: dimer Hinge 4, human IgG3 protein sequence:
EPKSCDTPPP CPRCP
SEQ ID NO: 1 61 C22 neoepitope amino acid sequence:
QIETQQRKFK ASRASILSEM KMLKEKR
SEQ ID NO: 62, C23 neoepitope amino acid sequence:
VILPQAPSGP SYATYLQPAQ AQMLTPP
SEQ ID NO: 63, C25 neoepitope amino acid sequence
DTLSAMSNPR AMQVLLQIQQ GLQTLAT
SEQ ID NO: 64, C30 neoepitope amino acid sequence;
DGQLELLAQG ALDNALSSMG ALHALPR
SEQ ID NO: 65, C38 neoepitope amino acid sequence;
RLHWKLLAS ALSTNAAALT QELLVLD
SEQ ID NO: 66, Alternative GS-linker:
GGGSS
SEQ ID NO: 67, C37 neoepitope amino acid sequence
GEVPPQKLQA LQRALQSEFC NAVREVY
SEQ ID NO: 68, EV85 neoepitope amino acid sequence
KKFMERDPDE LRFNTIALSA A
SEQ ID NO: 69, C40 neoepitope amino acid sequence
VTGTHKMSLG FTKARLLRLR NPWGRVE
SEQ ID NO: 70, C41 neoepitope amino acid sequence
LWTFSIYLES VAIMPQLFMV SKTGEAE
SEQ ID NO: 71, C29 neoepitope amino acid sequence
LHSGQNHLKE MAISVLEARA CAAAGQS
SEQ ID NO: 72, EV22 neoepitope amino acid sequence
GSLFGSSRVQ YVVNPAVKIV FLNIDPS
SEQ ID NO: 73, EV105 neoepitope amino acid sequence
PPPGLAAYTA KMATANGSKK AERQKFS
SEQ ID NO: 74, AA427 neoepitope amino acid sequence
VCNVKLLHRV LVADVNALQG MAAIGQR
SEQ ID NO: 75, pTVG4 vector backbone:
tggccattgc atacgttgta tccatatcat aatatgtaca tttatattgg ctcatgtcca
acattaccgc catgttgaca ttgattattg actagttatt aatagtaatc aattacgggg
tcattagttc atagcccata tatggagttc cgcgttacat aacttacggt aaatggcccg
cctggctgac cgcccaacga cccccgccca ttgacgtcaa taatgacgta tgttcccata
gtaacgccaa tagggacttt ccattgacgt caatgggtgg agtatttacg gtaaactgcc
cacttggcag tacatcaagt gtatcatatg ccaagtacgc cccctattga cgtcaatgac
ggtaaatggc ccgcctggca ttatgcccag tacatgacct tatgggactt tcctacttgg
cagtacatct acgtattagt catcgctatt accatggtga tgcggttttg gcagtacatc
aatgggcgtg gatagcggtt tgactcacgg ggatttccaa gtctccaccc cattgacgtc
aatgggagtt tgttttggca ccaaaatcaa cgggactttc caaaatgtcg taacaactcc
gccccattga cgcaaatggg cggtaggcgt gtacggtggg aggtctatat aagcagagct
cgtttagtga accgtcagat cgcctggaga cgccatccac gctgttttga cctccataga
agacaccggg accgatccag cctccgcggc cgggaacggt gcattggaac gcggattccc
cgtgccaaga gtgacgtaag taccgcctat agactctata ggcacacccc tttggctctt
atgcatgcta tactgttttt ggcttggggc ctatacaccc ccgcttcctt atgctatagg
tgatggtata gcttagccta taggtgtggg ttattgacca ttattgacca ctccaacggt
ggagggcagt gtagtctgag cagtactcgt tgctgccgcg cgcgccacca gacataatag
ctgacagact aacagactgt tcctttccat gggtcttttc tgcagtcacc gtcgtcgacg
gtatcgataa gcttgatatc gaattcacgt gggcccggta ccgtatactc tagagcggcc
gcggatccag atctaacgac aaaacgacaa aacgacaagg cgccagatct ggcgtttcgt
tttgtcgttt tgtcgttaga tctttttccc tctgccaaaa attatgggga catcatgaag
ccccttgagc atctgacttc tggctaataa aggaaattta ttttcattgc aatagtgtgt
tggaattttt tgtgtctctc actcggaagg acatatggga gggcaaatca tttaaaacat
cagaatgagt atttggttta gagtttggca acatatgccc attcttccgc ttcctcgctc
actgactcgc tgcgctcggt cgttcggctg cggcgagcgg tatcagctca ctcaaaggcg
gtaatacggt tatccacaga atcaggggat aacgcaggaa agaacatgtg agcaaaaggc
cagcaaaagg ccaggaaccg taaaaaggcc gcgttgctgg cgtttttcca taggctccgc
ccccctgacg agcatcacaa aaatcgacgc tcaagtcaga ggtggcgaaa cccgacagga
ctataaagat accaggcgtt tccccctgga agctccctcg tgcgctctcc tgttccgacc
ctgccgctta ccggatacct gtccgccttt ctcccttcgg gaagcgtggc gctttctcat
agctcacgct gtaggtatct cagttcggtg taggtcgttc gctccaagct gggctgtgtg
cacgaacccc ccgttcagcc cgaccgctgc gccttatccg gtaactatcg tcttgagtcc
aacccggtaa gacacgactt atcgccactg gcagcagcca ctggtaacag gattagcaga
gcgaggtatg taggcggtgc tacagagttc ttgaagtggt ggcctaacta cggctacact
agaagaacag tatttggtat ctgcgctctg ctgaagccag ttaccttcgg aaaaagagtt
ggtagctctt gatccggcaa acaaaccacc gctggtagcg gtggtttttt tgtttgcaag
cagcagatta cgcgcagaaa aaaaggatct caagaagatc ctttgatctt ttctacgggg
tctgacgctc agtggaacga aaactcacgt taagggattt tggtcatgag attatcaaaa
aggatcttca cctagatcct tttaaattaa aaatgaagtt ttaaatcaat ctaaagtata
tatgagtaaa cttggtctga cagttaccaa tgcttaatca gtgaggcacc tatctcagcg
atctgtctat ttcgttcatc catagttgcc tgactcgggg ggggggggcg ctgaggtctg
cctcgtgaag aaggtgttgc tgactcatac caggcctgaa tcgccccatc atccagccag
aaagtgaggg agccacggtt gatgagagct ttgttgtagg tggaccagtt ggtgattttg
aacttttgct ttgccacgga acggtctgcg ttgtcgggaa gatgcgtgat ctgatccttc
aactcagcaa aagttcgatt tattcaacaa agccgccgtc ccgtcaagtc agcgtaatgc
tctgccagtg ttacaaccaa ttaaccaatt ctgattagaa aaactcatcg agcatcaaat
gaaactgcaa tttattcata tcaggattat caataccata tttttgaaaa agccgtttct
gtaatgaagg agaaaactca ccgaggcagt tccataggat ggcaagatcc tggtatcggt
ctgcgattcc gactcgtcca acatcaatac aacctattaa tttcccctcg tcaaaaataa
ggttatcaag tgagaaatca ccatgagtga cgactgaatc cggtgagaat ggcaaaagct
tatgcatttc tttccagact tgttcaacag gccagccatt acgctcgtca tcaaaatcac
tcgcatcaac caaaccgtta ttcattcgtg attgcgcctg agcgagacga aatacgcgat
cgctgttaaa aggacaatta caaacaggaa tcgaatgcaa ccggcgcagg aacactgcca
gcgcatcaac aatattttca cctgaatcag gatattcttc taatacctgg aatgctgttt
tcccggggat cgcagtggtg agtaaccatg catcatcagg agtacggata aaatgcttga
tggtcggaag aggcataaat tccgtcagcc agtttagtct gaccatctca tctgtaacat
cattggcaac gctacctttg ccatgtttca gaaacaactc tggcgcatcg ggcttcccat
acaatcgata gattgtcgca cctgattgcc cgacattatc gcgagcccat ttatacccat
ataaatcagc atccatgttg gaatttaatc gcggcctcga gcaagacgtt tcccgttgaa
tatggctcat aacacccctt gtattactgt ttatgtaagc agacagtttt attgttcatg
atgatatatt tttatcttgt gcaatgtaac atcagagatt ttgagacaca acgtggcttt
cccccccccc ccattattga agcatttatc agggttattg tctcatgagc ggatacatat
ttgaatgtat ttagaaaaat aaacaaatag gggttccgcg cacatttccc cgaaaagtgc
cacctgacgt ctaagaaacc attattatca tgacattaac ctataaaaat aggcgtatca
cgaggccctt tcgtctcgcg cgtttcggtg atgacggtga aaacctctga cacatgcagc
tcccggagac ggtcacagct tgtctgtaag cggatgccgg gagcagacaa gcccgtcagg
gcgcgtcagc gggtgttggc gggtgtcggg gctggcttaa ctatgcggca tcagagcaga
ttgtactgag agtgcaccat atgcggtgtg aaataccgca cagatgcgta aggagaaaat
accgcatcag attggctat

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A method for the treatment of a neoplasm, such as a malignant neoplasm or for inducing a therapeutic or ameliorating immune response against such neoplasm, in a mammalian patient, wherein the neoplasm exhibits epitopes, such as T-cell epitopes (neo-epitopes) that are not exhibited by non-neoplastic cells in the patient, the method comprising administering an immunogenically effective amount of a composition comprising
A) an expression vector, which comprises a sequence of nucleotides encoding a fusion polypeptide, said fusion polypeptide comprising
i) at least one antigenic unit, which comprises a sequence of amino acids of at least one neo-epitope of a patent's neoplastic cells;
ii) at least one antigen presented cell (APC) targeting unit:
iii) optionally a multimerization unit, such as a dimerization unit, which unit provides for the multimerization of said fusion polypeptide to comprise two or more antigenic units and two or more antigen presenting cell(APC) targeting units,
wherein the APC targeting unit consists of or comprises a ligand selected from chemokine ligand 19 (CCL19) and chemokine ligand 21 (CCL21), or
B) a system of at least two expression constructs comprising i) a first expression construct comprising a sequence of nucleotides encoding at least one antigenic unit, which antigenic unit comprises a sequence of amino acids of at least one neo-epitope of a patient's neoplastic cells, and iii a second expression construct comprising a sequence of nucleotides encoding at least one antigen presenting cell (APC) targeting unit,
wherein the APC targeting unit consists of or comprises a ligand selected from chemokine ligand 19 (CCL19) and chemokine ligand 21 (CCL2 I),
whereby somatic cells in the patient are brought to express the sequence of nucleotides contained within the expression vector; the method optionally further comprising administering a pharmaceutically acceptable carrier, diluent, or excipient.

28. The method according to claim 27, wherein the patient is a human being.

29. The method according to claim 27, wherein the immunogenically effective amount of a composition is administered parenterally, such as via the intramuscular route, the intradermal route, transdermal route, the subcutaneous route, the intravenous route, the intra-arterial route, the intrathecal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

30. The method according to claim 27, wherein the pharmaceutically acceptable carrier, diluent, or excipient is an aqueous buttered solution.

31. The method according to claim 30, wherein the aqueous buffered solution is Tyrode's buffer.

32. The method according to claim 31, wherein the Trode's buffer has the composition 140 mM NaCl, 6 mM KCl, 3 mM CaCl₂, 2 mM MgCl₂, 10 mM 4-(2-hydroxyethyl)-1-piperazineethanesulfonic acid (Hepes) pH 7.4, and 10 mM glucose.

33. The method according to claim 31, wherein the concentration of Tyrode's buffer is about 35% v/v.

34. The method according to claim 30, where the buffer is PBS.

35. The method according to claim 27, wherein the method comprises administering an immunogenically effective amount of a composition comprising at least one expression vector as defined in any one of claims 10-15 with an effective dosage between 0.1 μg and 25 mg of the expression vector, such as between 0.5 μg and 20 mg, between 5 μg and 15 mg, between 50 μg and 10 mg, and between 500 μg and 8 tug, in particular about 0.0001, about 0.0005, about 0.001, about 0.005, about 0.01, about 0.05, about 0.1, about 0.5, about 1, about 2, about 3, about 4, about 5, about 6, about 7 and about 8 mg.

36. The method according to claim 27, wherein the immunogenically effective amount of said composition further comprises an effective amount of an amphiphilic block co-polymers comprising blocks of polyethylene oxide) and polypropylene oxide).

37. The method according to claim 27, wherein the APC targeting unit consists of or comprises an antibody binding region with specificity for target surface molecules on antigen presenting cells, such as HLA, HLA-DP, CD14, CD4O; or Toll-like receptor, such as Toll-like receptor 2; ligands, such as soluble CD40 ligand; CLEC9A Fv fragment, DEC205 Fv fragment, natural ligands like chemokines, such as a chemokine of the CC chemokine family, such as any one selected from chemokine ligand 3, chemokine ligand 4, chemokine ligand 5, chemokine ligand 19, chemokine ligand 20, chemokine ligand 21, or similar; or a chemokine of the CXC chemokine family, such as any one selected from chemokine (C-X-C motif) ligand 1 (CXCL1), or similar, RANTES or bacterial antigens, such as flagellin or a part thereof.

38. The method according to claim 27, wherein the APC targeting unit consists of or comprises a ligand, such as soluble CD40 ligand; CLEC9A peptide ligand, DEC205, FLT3L, GM-CSF, natural ligands like chemokines, such as a chemokine of the CC chemokine family, such as any one selected from chemokine ligand 3, chemokine ligand 4, chemokine ligand 5, chemokine ligand 19, chemokine ligand 20, chemokine ligand 21, or similar; or a chemokine of the CXC chemokine family, such as any one selected from chemokine (C-X-C motif) ligand 1 (CXCL1), or similar, such as RANTES or Chernokine ligand 3 (CCL3/MIP-la) or CCL19-, or bacterial antigens, such as flagellin or a part thereof.

39. The method according to claim 27, wherein the antigenic unit is connected to the targeting unit through a linker, such as GS linker, such as linker with the amino acid sequence GSGSGSGSGS (SEQ ID NO: 13), or a linker derived from an immunoglobulin molecule (Ig), such as IgG, such as a linker which contributes to the multimerization through the formation of an interchain covalent bond.

40. The method according to claim 39, wherein the linker is or comprises a hinge region, such as an Ig, such as an IgG-derived hinge region and contributes to the multimerization through the formation of an interchain covalent bond, such as a disulfide bridge.

41. The method according to claim 39, wherein the linker comprises a carboxyterminal C domain (CH3 domain), such as the carboxyterminal C domain of Ig (Cγ3 domain), or a sequence that is substantially homologous to said C domain, such as the CH3 domain of IgG3.

42. The method according to claim 41, wherein the hinge and CH3 domain are connected by a sequence of amino acids GlyGlyGlySerSer (SEQ 1D NO: 13), such as in triplicate sequence of the amino acids GlyGlyGlySerSer.

43. The method according to claim 39, wherein the linker comprises a dimerization motif or any other multimerization domain, which participate in the multimerization through hydrophobic interactions, such as through a CH3 domain.

44. The method according to claim 39, wherein the linker comprises a hinge region comprising h1+h4 or h4 derived from IgG, such as an IgG2 or IgG3.

45. The method according to claim 27, wherein the at least one antigenic unit consist of or comprises at least or about 5, such as at least or about 10, at least or about 15, at least or about 20, at least or about 25, and at least or about 30 neo-epitopes.

46. The method according to claim 27, wherein the at least one neo-epitope includes a neo-epitope, which exhibits an MHC binding stability, which is above average, such as in the top quartile, among neo-epitopes identified in the neoplastic cells.

47. The method according to claim 27, which multimerization, such as a dimerization unit, enables the formation of dimers, trimers, tetramers, pentamers, or multimers of higher order.

48. The method according to claim 27, wherein the first expression construct comprising a sequence of nucleotides encoding at least one antigenic unit consists of or comprises at least or about 5, such as at least or about 10, at least or about 15, at least or about 20, at least or about 25, and at least or about 30 neo-epitopes.

49. The method according to claim 27, wherein the first expression construct in B comprises a sequence of nucleotides encoding at least one neo-epitope, which includes a neo-epitope, which exhibits an MHC binding stability, which is above average, such as in the top quartile, among neo-epitopes identified in the neoplastic cells.

50. The method according to claim 27, wherein the first expression construct comprising a sequence of nucleotides encoding at least one antigenic unit further comprises a sequence of nucleotides encoding a multimerization unit, such as a dimerization unit, which unit provides for the multimerization of said at least one antigenic unit.

51. The method according to claim 27, wherein the at least two expression constructs are expressed by the same expression vector, such as under the control of two different promotors.

52. The method according to claim 27, wherein the at least two expression cons are expressed by at least two different vectors.
